SEQUENCE LISTING (198 sequences)

SEQ ID NO: 1 - DNA, 118 bp, Artificial Sequence
  Synthetic
  misc_feature (57)..(57): n is g, c, or a
  misc_feature (58)..(58): n is a or c
  misc_feature (59)..(59): n is a, c, or t
  misc_feature (60)..(60): n is c or t
  misc_feature (61)..(61): n is t or a
  misc_feature (62)..(62): n is g or a
  misc_feature (63)..(63): n is a or g
  misc_feature (64)..(64): n is t, g, or a
  misc_feature (65)..(65): n is a, g, or c
aacgctgaagcgctgggggcaaattgtgcaatttgcggccaatgtttgtaatcagcnnnnnnnnnccaaggaaattttggggaccaggaactaatcagacaaggaactgattacaaac

SEQ ID NO: 2 - DNA, 289 bp, Artificial Sequence
  Synthetic
tccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagact

SEQ ID NO: 3 - DNA, 21 bp, Artificial Sequence
  Synthetic
tccagatgaggatgaagaaga

SEQ ID NO: 4 - DNA, 21 bp, Artificial Sequence
  Synthetic
agtctgaacaactggtgtaag

SEQ ID NO: 5 - DNA, 41 bp, Artificial Sequence
  Synthetic
agagcagcagaagtggcacaggtgattgtgaagaagaagag

SEQ ID NO: 6 - DNA, 40 bp, Artificial Sequence
  Synthetic
tcaacctgaagaagagcaagaactgattgtcctcactgcc

SEQ ID NO: 7 - DNA, 22 bp, Artificial Sequence
  Synthetic
ctcatattgagttgatggctca

SEQ ID NO: 8 - DNA, 22 bp, Artificial Sequence
  Synthetic
acaaactgttggtcaacaagac

SEQ ID NO: 9 - DNA, 46 bp, Artificial Sequence
  Synthetic
agagcagcagaagtggcacagcttaggtgattgtgaagaagaagag

SEQ ID NO: 10 - DNA, 51 bp, Artificial Sequence
  Synthetic
agagcagcagaagtggcacagcttacactaggtgattgtgaagaagaagag

SEQ ID NO: 11 - DNA, 61 bp, Artificial Sequence
  Synthetic
agagcagcagaagtggcacagcttacactcaatgatccgaggtgattgtgaagaagaagag

SEQ ID NO: 12 - DNA, 74 bp, Artificial Sequence
  Synthetic
agagcagcagaagtggcacagcttacactcaatgatccgatatggagcccagaggtgattgtgaagaagaagag

SEQ ID NO: 13 - DNA, 217 bp, Artificial Sequence
  Synthetic
tggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgca

SEQ ID NO: 14 - DNA, 19 bp, Artificial Sequence
  Synthetic
tggctactaccgaagagct

SEQ ID NO: 15 - DNA, 20 bp, Artificial Sequence
  Synthetic
tgcagcattgttagcaggat

SEQ ID NO: 16 - DNA, 41 bp, Artificial Sequence
  Synthetic
tctggcccagttcctaggtagtccagacgaattcgtggtgg

SEQ ID NO: 17 - DNA, 40 bp, Artificial Sequence
  Synthetic
agacggcatcatatgggttgcacgggtgccaatgtgatct

SEQ ID NO: 18 - DNA, 25 bp, Artificial Sequence
  Synthetic
ggactgagatctttcattttaccgt

SEQ ID NO: 19 - DNA, 21 bp, Artificial Sequence
  Synthetic
actgagggagccttgaataca

SEQ ID NO: 20 - DNA, 46 bp, Artificial Sequence
  Synthetic
tctggcccagttcctaggtagtagcttccagacgaattcgtggtgg

SEQ ID NO: 21 - DNA, 51 bp, Artificial Sequence
  Synthetic
tctggcccagttcctaggtagtagcttacactccagacgaattcgtggtgg

SEQ ID NO: 22 - DNA, 61 bp, Artificial Sequence
  Synthetic
tctggcccagttcctaggtagtagcttacactcaatgatccgccagacgaattcgtggtgg

SEQ ID NO: 23 - DNA, 74 bp, Artificial Sequence
  Synthetic
tctggcccagttcctaggtagtagcttacactcaatgatccgatatggagcccagccagacgaattcgtggtgg

SEQ ID NO: 24 - DNA, 45 bp, Artificial Sequence
  Synthetic
agacggcatcatatgggttgcaagagccgggtgccaatgtgatct

SEQ ID NO: 25 - DNA, 50 bp, Artificial Sequence
  Synthetic
agacggcatcatatgggttgcaagagccgttgcgggtgccaatgtgatct

SEQ ID NO: 26 - DNA, 60 bp, Artificial Sequence
  Synthetic
agacggcatcatatgggttgcaagagccgttgcagggattaacgggtgccaatgtgatct

SEQ ID NO: 27 - DNA, 73 bp, Artificial Sequence
  Synthetic
agacggcatcatatgggttgcaagagccgttgcagggattaaggaacacgtccaccgggtgccaatgtgatct

SEQ ID NO: 28 - DNA, 229 bp, Artificial Sequence
  Synthetic
aacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttc

SEQ ID NO: 29 - DNA, 18 bp, Artificial Sequence
  Synthetic
aacacaagctttcggcag

SEQ ID NO: 30 - DNA, 22 bp, Artificial Sequence
  Synthetic
gaaatttggatctttgtcatcc

SEQ ID NO: 31 - DNA, 41 bp, Artificial Sequence
  Synthetic
tgcggccaatgtttgtaatcagccaaggaaattttggggac

SEQ ID NO: 32 - DNA, 39 bp, Artificial Sequence
  Synthetic
cgcattggcatggaagtcactttgatggcacctgtgtag

SEQ ID NO: 33 - DNA, 19 bp, Artificial Sequence
  Synthetic
ttccttgtctgattagttc

SEQ ID NO: 34 - DNA, 18 bp, Artificial Sequence
  Synthetic
accttcgggaacgtggtt

SEQ ID NO: 35 - DNA, 46 bp, Artificial Sequence
  Synthetic
tgcggccaatgtttgtaatcagagcttccaaggaaattttggggac

SEQ ID NO: 36 - DNA, 51 bp, Artificial Sequence
  Synthetic
tgcggccaatgtttgtaatcagagcttacactccaaggaaattttggggac

SEQ ID NO: 37 - DNA, 61 bp, Artificial Sequence
  Synthetic
tgcggccaatgtttgtaatcagagcttacactcaatgatccgccaaggaaattttggggac

SEQ ID NO: 38 - DNA, 74 bp, Artificial Sequence
  Synthetic
tgcggccaatgtttgtaatcagagcttacactcaatgatccgatatggagcccagccaaggaaattttggggac

SEQ ID NO: 39 - DNA, 118 bp, Artificial Sequence
  Synthetic
aacgctgaagcgctgggggcaaattgtgcaatttgcggccaatgtttgtaatcagagcttacactccaaggaaattttggggaccaggaactaatcagacaaggaactgattacaaac

SEQ ID NO: 40 - DNA, 10 bp, Artificial Sequence
  Synthetic
cagatgtccc

SEQ ID NO: 41 - DNA, 10 bp, Artificial Sequence
  Synthetic
atcgctgtcg

SEQ ID NO: 42 - DNA, 10 bp, Artificial Sequence
  Synthetic
gattatgtcc

SEQ ID NO: 43 - DNA, 10 bp, Artificial Sequence
  Synthetic
tgcttagtcc

SEQ ID NO: 44 - DNA, 10 bp, Artificial Sequence
  Synthetic
ttagaagtcc

SEQ ID NO: 45 - DNA, 10 bp, Artificial Sequence
  Synthetic
ctttccgtcc

SEQ ID NO: 46 - DNA, 12 bp, Artificial Sequence
  Synthetic
  misc_feature (1)..(12): n is a, c, g, or t
nnnnnnnnnnnn

SEQ ID NO: 47 - DNA, 51 bp, Artificial Sequence
  Synthetic - barcoded forward inner primer where N can be any nucleotide and collectively denote a specific barcode sequence
  misc_feature (23)..(32): n is a, c, g, or t
tctggcccagttcctaggtagtnnnnnnnnnnccagacgaattcgtggtgg

SEQ ID NO: 48 - DNA, 40 bp, Artificial Sequence; identical to SEQ ID NO: 17
  Synthetic - barcoded backward inner primer

SEQ ID NO: 49 - DNA, 19 bp, Artificial Sequence
  Synthetic - primer (B-F3)
tggctactaccgaagagct

SEQ ID NO: 50 - DNA, 20 bp, Artificial Sequence
  Synthetic - primer (B-B3)
tgcagcattgttagcaggat

SEQ ID NO: 51 - DNA, 25 bp, Artificial Sequence
  Synthetic - primer (B-LF)
ggactgagatctttcattttaccgt

SEQ ID NO: 52 - DNA, 21 bp, Artificial Sequence
  Synthetic - primer (Bl-LB)
actgagggagccttgaataca

SEQ ID NO: 53 - DNA, 56 bp, Artificial Sequence
  Synthetic - primer-PCR B-fwd-10 primer
acactctttccctacacgacgctcttccgatctccgtctttgttagcaccataggg

SEQ ID NO: 54 - DNA, 56 bp, Artificial Sequence
  Synthetic - primer PCR-B-rev-12
tgactggagttcagacgtgtgctcttccgatctaccatcttggactgagatctttc

SEQ ID NO: 55 - DNA, 62 bp, Artificial Sequence
  Synthetic - pool-specific forward barcoding primer, N′s can be any nucleotide and collectively denote a specific barcode sequence
  misc_feature (30)..(39): n is a, c, g, or t
aatgatacggcgaccaccgagatctacacnnnnnnnnnnacactctttccctacacgacgct

SEQ ID NO: 56 - DNA, 58 bp, Artificial Sequence
  Synthetic - poll specific reverse barcoding primer, N can be any nucleotide and collectively N′s denote a specific barcode sequence
  misc_feature (25)..(34): n is a, c, g, or t
caagcagaagacggcatacgagatnnnnnnnnnngtgactggagttcagacgtgtgct

SEQ ID NO: 57 - DNA, 21 bp, Artificial Sequence
  Synthetic - primer, where relevant N is any nucleotide and collectively denotes a specific barcode seuqence
tccagatgaggatgaagaaga

SEQ ID NO: 58 - DNA, 21 bp, Artificial Sequence
  Synthetic - primer, where relevant N is any nucleotide and collectively denotes a specific barcode seuqence
agtctgaacaactggtgtaag

SEQ ID NO: 59 - DNA, 51 bp, Artificial Sequence
  Synthetic - primer, where relevant N is any nucleotide and collectively denotes a specific barcode seuqence
  misc_feature (20)..(29): n is a, c, g, or t
agagcagcagaagtggcacnnnnnnnnnnaggtgattgtgaagaagaagag

SEQ ID NO: 60 - DNA, 40 bp, Artificial Sequence; identical to SEQ ID NO: 6
  Synthetic - primer, where relevant N is any nucleotide and collectively denotes a specific barcode seuqence

SEQ ID NO: 61 - DNA, 22 bp, Artificial Sequence
  Synthetic - primer, where relevant N is any nucleotide and collectively denotes a specific barcode seuqence
ctcatattgagttgatggctca

SEQ ID NO: 62 - DNA, 22 bp, Artificial Sequence
  Synthetic - primer, where relevant N is any nucleotide and collectively denotes a specific barcode seuqence
acaaactgttggtcaacaagac

SEQ ID NO: 63 - DNA, 19 bp, Artificial Sequence
  Synthetic - primer, where relevant N is any nucleotide and collectively denotes a specific barcode seuqence
tggctactaccgaagagct

SEQ ID NO: 64 - DNA, 20 bp, Artificial Sequence
  Synthetic - primer, where relevant N is any nucleotide and collectively denotes a specific barcode seuqence
tgcagcattgttagcaggat

SEQ ID NO: 65 - DNA, 51 bp, Artificial Sequence; identical to SEQ ID NO: 47
  Synthetic - primer, where relevant N is any nucleotide and collectively denotes a specific barcode seuqence
  misc_feature (23)..(32): n is a, c, g, or t

SEQ ID NO: 66 - DNA, 40 bp, Artificial Sequence; identical to SEQ ID NO: 17
  Synthetic - primer, where relevant N is any nucleotide and collectively denotes a specific barcode seuqence

SEQ ID NO: 67 - DNA, 25 bp, Artificial Sequence
  Synthetic - primer, where relevant N is any nucleotide and collectively denotes a specific barcode seuqence
ggactgagatctttcattttaccgt

SEQ ID NO: 68 - DNA, 21 bp, Artificial Sequence
  Synthetic - primer, where relevant N is any nucleotide and collectively denotes a specific barcode seuqence
actgagggagccttgaataca

SEQ ID NO: 69 - DNA, 56 bp, Artificial Sequence; identical to SEQ ID NO: 53
  Synthetic - primer, where relevant N is any nucleotide and collectively denotes a specific barcode seuqence

SEQ ID NO: 70 - DNA, 56 bp, Artificial Sequence; identical to SEQ ID NO: 54
  Synthetic - primer, where relevant N is any nucleotide and collectively denotes a specific barcode seuqence

SEQ ID NO: 71 - DNA, 18 bp, Artificial Sequence
  Synthetic - primer, where relevant N is any nucleotide and collectively denotes a specific barcode seuqence
aacacaagctttcggcag

SEQ ID NO: 72 - DNA, 22 bp, Artificial Sequence
  Synthetic - primer, where relevant N is any nucleotide and collectively denotes a specific barcode seuqence
gaaatttggatctttgtcatcc

SEQ ID NO: 73 - DNA, 51 bp, Artificial Sequence
  Synthetic - primer, where relevant N is any nucleotide and collectively denotes a specific barcode seuqence
  misc_feature (23)..(32): n is a, c, g, or t
tgcggccaatgtttgtaatcagnnnnnnnnnnccaaggaaattttggggac

SEQ ID NO: 74 - DNA, 39 bp, Artificial Sequence; identical to SEQ ID NO: 32
  Synthetic - primer, where relevant N is any nucleotide and collectively denotes a specific barcode seuqence

SEQ ID NO: 75 - DNA, 19 bp, Artificial Sequence
  Synthetic - primer, where relevant N is any nucleotide and collectively denotes a specific barcode seuqence
ttccttgtctgattagttc

SEQ ID NO: 76 - DNA, 18 bp, Artificial Sequence
  Synthetic - primer, where relevant N is any nucleotide and collectively denotes a specific barcode seuqence
accttcgggaacgtggtt

SEQ ID NO: 77 - DNA, 56 bp, Artificial Sequence
  Synthetic - primer, where relevant N is any nucleotide and collectively denotes a specific barcode seuqence
acactctttccctacacgacgctcttccgatctaacgctgaagcgctgggggcaaa

SEQ ID NO: 78 - DNA, 56 bp, Artificial Sequence
  Synthetic - primer, where relevant N is any nucleotide and collectively denotes a specific barcode seuqence
tgactggagttcagacgtgtgctcttccgatctgtttgtaatcagttccttgtctg

SEQ ID NO: 79 - DNA, 21 bp, Artificial Sequence
  Synthetic - Primer Binding Site in SARS CoV-2 Genome, where relevant N is any nucleotide and collectively denotes a specific barcode sequence
tccagatgaggatgaagaaga

SEQ ID NO: 80 - DNA, 21 bp, Artificial Sequence
  Synthetic - Primer Sequence (5′-3′), where relevant N is any nucleotide and collectively denotes a specific barcode sequence
tccagatgaggatgaagaaga

SEQ ID NO: 81 - DNA, 21 bp, Artificial Sequence
  Synthetic - primer (A-F3), where relevant N is any nucleotide and collectively denotes a specific barcode sequence
tccagatgaggatgaagaaga

SEQ ID NO: 82 - DNA, 21 bp, Artificial Sequence
  Synthetic - Primer Binding Site in SARS CoV-2 Genome, where relevant N is any nucleotide and collectively denotes a specific barcode sequence
cttacaccagttgttcagact

SEQ ID NO: 83 - DNA, 21 bp, Artificial Sequence
  Synthetic - Primer Sequence (5′-3′), where relevant N is any nucleotide and collectively denotes a specific barcode sequence
agtctgaacaactggtgtaag

SEQ ID NO: 84 - DNA, 21 bp, Artificial Sequence
  Synthetic - primer (A-B3), where relevant N is any nucleotide and collectively denotes a specific barcode sequence
agtctgaacaactggtgtaag

SEQ ID NO: 85 - DNA, 22 bp, Artificial Sequence
  Synthetic - Primer Binding Site in SARS CoV-2 Genome, where relevant N is any nucleotide and collectively denotes a specific barcode sequence
aggtgattgtgaagaagaagag

SEQ ID NO: 86 - DNA, 22 bp, Artificial Sequence
  Synthetic - Primer Sequence (5′-3′), where relevant N is any nucleotide and collectively denotes a specific barcode sequence
aggtgattgtgaagaagaagag

SEQ ID NO: 87 - DNA, 51 bp, Artificial Sequence; identical to SEQ ID NO: 59
  Synthetic - primer (A-FIP), where relevant N is any nucleotide and collectively denotes a specific barcode sequence
  misc_feature (20)..(29): n is a, c, g, or t

SEQ ID NO: 88 - DNA, 22 bp, Artificial Sequence
  Synthetic - Primer Binding Site in SARS CoV-2 Genome, where relevant N is any nucleotide and collectively denotes a specific barcode sequence
tcaacctgaagaagagcaagaa

SEQ ID NO: 89 - DNA, 22 bp, Artificial Sequence
  Synthetic - Primer Sequence (5′-3′), where relevant N is any nucleotide and collectively denotes a specific barcode sequence
tcaacctgaagaagagcaagaa

SEQ ID NO: 90 - DNA, 40 bp, Artificial Sequence; identical to SEQ ID NO: 6
  Synthetic - primer (A-BIP), where relevant N is any nucleotide and collectively denotes a specific barcode sequence

SEQ ID NO: 91 - DNA, 18 bp, Artificial Sequence
  Synthetic - Primer Binding Site in SARS CoV-2 Genome, where relevant N is any nucleotide and collectively denotes a specific barcode sequence
ggcagtgaggacaatcag

SEQ ID NO: 92 - DNA, 18 bp, Artificial Sequence
  Synthetic - Primer Sequence (5′-3′), where relevant N is any nucleotide and collectively denotes a specific barcode sequence
ctgattgtcctcactgcc

SEQ ID NO: 93 - DNA, 40 bp, Artificial Sequence; identical to SEQ ID NO: 6
  Synthetic - primer (A-BIP), where relevant N is any nucleotide and collectively denotes a specific barcode sequence

SEQ ID NO: 94 - DNA, 22 bp, Artificial Sequence
  Synthetic - Primer Binding Site in SARS CoV-2 Genome, where relevant N is any nucleotide and collectively denotes a specific barcode sequence
tgagccatcaactcaatatgag

SEQ ID NO: 95 - DNA, 22 bp, Artificial Sequence
  Synthetic - Primer Sequence (5′-3′), where relevant N is any nucleotide and collectively denotes a specific barcode sequence
ctcatattgagttgatggctca

SEQ ID NO: 96 - DNA, 22 bp, Artificial Sequence
  Synthetic - primer (A-LF), where relevant N is any nucleotide and collectively denotes a specific barcode sequence
ctcatattgagttgatggctca

SEQ ID NO: 97 - DNA, 22 bp, Artificial Sequence
  Synthetic - Primer Binding Site in SARS CoV-2 Genome, where relevant N is any nucleotide and collectively denotes a specific barcode sequence
acaaactgttggtcaacaagac

SEQ ID NO: 98 - DNA, 22 bp, Artificial Sequence
  Synthetic - Primer Sequence (5′-3′), where relevant N is any nucleotide and collectively denotes a specific barcode sequence
acaaactgttggtcaacaagac

SEQ ID NO: 99 - DNA, 22 bp, Artificial Sequence
  Synthetic - primer (A-LB), where relevant N is any nucleotide and collectively denotes a specific barcode sequence
acaaactgttggtcaacaagac

SEQ ID NO: 100 - DNA, 23 bp, Artificial Sequence
  Synthetic - Primer Binding Site in SARS CoV-2 Genome, where relevant N is any nucleotide and collectively denotes a specific barcode sequence
ggtactgaagatgattaccaagg

SEQ ID NO: 101 - DNA, 23 bp, Artificial Sequence
  Synthetic - Primer Sequence (5′-3′), where relevant N is any nucleotide and collectively denotes a specific barcode sequence
ccttggtaatcatcttcagtacc

SEQ ID NO: 102 - DNA, 56 bp, Artificial Sequence
  Synthetic - primer (miseq A f wd 10), where relevant N is any nucleotide and collectively denotes a specific barcode sequence
acactctttccctacacgacgctcttccgatctccttggtaatcatcttcagtacc

SEQ ID NO: 103 - DNA, 11 bp, Artificial Sequence
  Synthetic - Primer Binding Site in SARS CoV-2 Genome, where relevant N is any nucleotide and collectively denotes a specific barcode sequence
aggacaatcag

SEQ ID NO: 104 - DNA, 11 bp, Artificial Sequence
  Synthetic - Primer Sequence (5′-3′), where relevant N is any nucleotide and collectively denotes a specific barcode sequence
aggacaatcag

SEQ ID NO: 105 - DNA, 56 bp, Artificial Sequence
  Synthetic - primer (miseq A r ev 12), where relevant N is any nucleotide and collectively denotes a specific barcode sequence
tgactggagttcagacgtgtgctcttccgatctaggacaatcagttcttgctcttc

SEQ ID NO: 106 - DNA, 12 bp, Artificial Sequence
  Synthetic - Primer Binding Site in SARS CoV-2 Genome, where relevant N is any nucleotide and collectively denotes a specific barcode sequence
gaagagcaagaa

SEQ ID NO: 107 - DNA, 12 bp, Artificial Sequence
  Synthetic - Primer Sequence (5′-3′), where relevant N is any nucleotide and collectively denotes a specific barcode sequence
ttcttgctcttc

SEQ ID NO: 108 - DNA, 56 bp, Artificial Sequence; identical to SEQ ID NO: 105
  Synthetic - primer (miseq A r ev 12), where relevant N is any nucleotide and collectively denotes a specific barcode sequence

SEQ ID NO: 109 - DNA, 19 bp, Artificial Sequence
  Synthetic - Primer Binding Site in SARS CoV-2 Genome, where relevant N is any nucleotide and collectively denotes a specific barcode sequence
tggctactaccgaagagct

SEQ ID NO: 110 - DNA, 19 bp, Artificial Sequence
  Synthetic - Primer Sequence (5′-3′), where relevant N is any nucleotide and collectively denotes a specific barcode sequence
tggctactaccgaagagct

SEQ ID NO: 111 - DNA, 19 bp, Artificial Sequence
  Synthetic - primer (B-F3), where relevant N is any nucleotide and collectively denotes a specific barcode sequence
tggctactaccgaagagct

SEQ ID NO: 112 - DNA, 20 bp, Artificial Sequence
  Synthetic - Primer Binding Site in SARS CoV-2 Genome, where relevant N is any nucleotide and collectively denotes a specific barcode sequence
atcctgctaacaatgctgca

SEQ ID NO: 113 - DNA, 20 bp, Artificial Sequence
  Synthetic - Primer Sequence (5′-3′), where relevant N is any nucleotide and collectively denotes a specific barcode sequence
tgcagcattgttagcaggat

SEQ ID NO: 114 - DNA, 20 bp, Artificial Sequence
  Synthetic - primer (B-B3), where relevant N is any nucleotide and collectively denotes a specific barcode sequence
tgcagcattgttagcaggat

SEQ ID NO: 115 - DNA, 19 bp, Artificial Sequence
  Synthetic - Primer Binding Site in SARS CoV-2 Genome, where relevant N is any nucleotide and collectively denotes a specific barcode sequence
ccagacgaattcgtggtgg

SEQ ID NO: 116 - DNA, 19 bp, Artificial Sequence
  Synthetic - Primer Sequence (5′-3′), where relevant N is any nucleotide and collectively denotes a specific barcode sequence
ccagacgaattcgtggtgg

SEQ ID NO: 117 - DNA, 51 bp, Artificial Sequence; identical to SEQ ID NO: 47
  Synthetic - primer (B-FIP), where relevant N is any nucleotide and collectively denotes a specific barcode sequence
  misc_feature (23)..(32): n is a, c, g, or t

SEQ ID NO: 118 - DNA, 22 bp, Artificial Sequence
  Synthetic - Primer Binding Site in SARS CoV-2 Genome, where relevant N is any nucleotide and collectively denotes a specific barcode sequence
agacggcatcatatgggttgca

SEQ ID NO: 119 - DNA, 22 bp, Artificial Sequence
  Synthetic - Primer Sequence (5′-3′), where relevant N is any nucleotide and collectively denotes a specific barcode sequence
agacggcatcatatgggttgca

SEQ ID NO: 120 - DNA, 40 bp, Artificial Sequence; identical to SEQ ID NO: 17
  Synthetic - primer (B-BIP), where relevant N is any nucleotide and collectively denotes a specific barcode sequence

SEQ ID NO: 121 - DNA, 18 bp, Artificial Sequence
  Synthetic - Primer Binding Site in SARS CoV-2 Genome, where relevant N is any nucleotide and collectively denotes a specific barcode sequence
agatcacattggcacccg

SEQ ID NO: 122 - DNA, 18 bp, Artificial Sequence
  Synthetic - Primer Sequence (5′-3′), where relevant N is any nucleotide and collectively denotes a specific barcode sequence
cgggtgccaatgtgatct

SEQ ID NO: 123 - DNA, 40 bp, Artificial Sequence; identical to SEQ ID NO: 17
  Synthetic - primer (B-BIP), where relevant N is any nucleotide and collectively denotes a specific barcode sequence

SEQ ID NO: 124 - DNA, 25 bp, Artificial Sequence
  Synthetic - Primer Binding Site in SARS CoV-2 Genome, where relevant N is any nucleotide and collectively denotes a specific barcode sequence
acggtaaaatgaaagatctcagtcc

SEQ ID NO: 125 - DNA, 25 bp, Artificial Sequence
  Synthetic - Primer Sequence (5′-3′), where relevant N is any nucleotide and collectively denotes a specific barcode sequence
ggactgagatctttcattttaccgt

SEQ ID NO: 126 - DNA, 25 bp, Artificial Sequence
  Synthetic - primer (B-LF), where relevant N is any nucleotide and collectively denotes a specific barcode sequence
ggactgagatctttcattttaccgt

SEQ ID NO: 127 - DNA, 21 bp, Artificial Sequence
  Synthetic - Primer Binding Site in SARS CoV-2 Genome, where relevant N is any nucleotide and collectively denotes a specific barcode sequence
actgagggagccttgaataca

SEQ ID NO: 128 - DNA, 21 bp, Artificial Sequence
  Synthetic - Primer Sequence (5′-3′), where relevant N is any nucleotide and collectively denotes a specific barcode sequence
actgagggagccttgaataca

SEQ ID NO: 129 - DNA, 21 bp, Artificial Sequence
  Synthetic - primer (B-LB), where relevant N is any nucleotide and collectively denotes a specific barcode sequence
actgagggagccttgaataca

SEQ ID NO: 130 - DNA, 23 bp, Artificial Sequence
  Synthetic - Primer Binding Site in SARS CoV-2 Genome, where relevant N is any nucleotide and collectively denotes a specific barcode sequence
ccctatggtgctaacaaagacgg

SEQ ID NO: 131 - DNA, 23 bp, Artificial Sequence
  Synthetic - Primer Sequence (5′-3′), where relevant N is any nucleotide and collectively denotes a specific barcode sequence
ccgtctttgttagcaccataggg

SEQ ID NO: 132 - DNA, 56 bp, Artificial Sequence; identical to SEQ ID NO: 53
  Synthetic - primer (miseq B f wd 10), where relevant N is any nucleotide and collectively denotes a specific barcode sequence

SEQ ID NO: 133 - DNA, 23 bp, Artificial Sequence
  Synthetic - Primer Binding Site in SARS CoV-2 Genome, where relevant N is any nucleotide and collectively denotes a specific barcode sequence
gaaagatctcagtccaagatggt

SEQ ID NO: 134 - DNA, 23 bp, Artificial Sequence
  Synthetic - Primer Sequence (5′-3′), where relevant N is any nucleotide and collectively denotes a specific barcode sequence
accatcttggactgagatctttc

SEQ ID NO: 135 - DNA, 56 bp, Artificial Sequence; identical to SEQ ID NO: 54
  Synthetic - primer (miseq B r ev 12), where relevant N is any nucleotide and collectively denotes a specific barcode sequence

SEQ ID NO: 136 - DNA, 18 bp, Artificial Sequence
  Synthetic - Primer Binding Site in SARS CoV-2 Genome, where relevant N is any nucleotide and collectively denotes a specific barcode sequence
aacacaagctttcggcag

SEQ ID NO: 137 - DNA, 18 bp, Artificial Sequence
  Synthetic - Primer Sequence (5′-3′), where relevant N is any nucleotide and collectively denotes a specific barcode sequence
aacacaagctttcggcag

SEQ ID NO: 138 - DNA, 18 bp, Artificial Sequence
  Synthetic - primer (C-F3), where relevant N is any nucleotide and collectively denotes a specific barcode sequence
aacacaagctttcggcag

SEQ ID NO: 139 - DNA, 22 bp, Artificial Sequence
  Synthetic - Primer Binding Site in SARS CoV-2 Genome, where relevant N is any nucleotide and collectively denotes a specific barcode sequence
ggatgacaaagatccaaatttc

SEQ ID NO: 140 - DNA, 22 bp, Artificial Sequence
  Synthetic - Primer Sequence (5′-3′), where relevant N is any nucleotide and collectively denotes a specific barcode sequence
gaaatttggatctttgtcatcc

SEQ ID NO: 141 - DNA, 22 bp, Artificial Sequence
  Synthetic - primer (C-B3), where relevant N is any nucleotide and collectively denotes a specific barcode sequence
gaaatttggatctttgtcatcc

SEQ ID NO: 142 - DNA, 19 bp, Artificial Sequence
  Synthetic - Primer Binding Site in SARS CoV-2 Genome, where relevant N is any nucleotide and collectively denotes a specific barcode sequence
ccaaggaaattttggggac

SEQ ID NO: 143 - DNA, 19 bp, Artificial Sequence
  Synthetic - Primer Sequence (5′-3′), where relevant N is any nucleotide and collectively denotes a specific barcode sequence
ccaaggaaattttggggac

SEQ ID NO: 144 - DNA, 51 bp, Artificial Sequence; identical to SEQ ID NO: 73
  Synthetic - primer (C-FIP), where relevant N is any nucleotide and collectively denotes a specific barcode sequence
  misc_feature (23)..(32): n is a, c, g, or t

SEQ ID NO: 145 - DNA, 20 bp, Artificial Sequence
  Synthetic - Primer Binding Site in SARS CoV-2 Genome, where relevant N is any nucleotide and collectively denotes a specific barcode sequence
cgcattggcatggaagtcac

SEQ ID NO: 146 - DNA, 20 bp, Artificial Sequence
  Synthetic - Primer Sequence (5′-3′), where relevant N is any nucleotide and collectively denotes a specific barcode sequence
cgcattggcatggaagtcac

SEQ ID NO: 147 - DNA, 39 bp, Artificial Sequence; identical to SEQ ID NO: 32
  Synthetic - primer (C-BIP), where relevant N is any nucleotide and collectively denotes a specific barcode sequence

SEQ ID NO: 148 - DNA, 19 bp, Artificial Sequence
  Synthetic - Primer Binding Site in SARS CoV-2 Genome, where relevant N is any nucleotide and collectively denotes a specific barcode sequence
ctacacaggtgccatcaaa

SEQ ID NO: 149 - DNA, 19 bp, Artificial Sequence
  Synthetic - Primer Sequence (5′-3′), where relevant N is any nucleotide and collectively denotes a specific barcode sequence
tttgatggcacctgtgtag

SEQ ID NO: 150 - DNA, 39 bp, Artificial Sequence; identical to SEQ ID NO: 32
  Synthetic - primer (C-BIP), where relevant N is any nucleotide and collectively denotes a specific barcode sequence

SEQ ID NO: 151 - DNA, 19 bp, Artificial Sequence
  Synthetic - Primer Binding Site in SARS CoV-2 Genome, where relevant N is any nucleotide and collectively denotes a specific barcode sequence
gaactaatcagacaaggaa

SEQ ID NO: 152 - DNA, 19 bp, Artificial Sequence
  Synthetic - Primer Sequence (5′-3′), where relevant N is any nucleotide and collectively denotes a specific barcode sequence
ttccttgtctgattagttc

SEQ ID NO: 153 - DNA, 19 bp, Artificial Sequence
  Synthetic - primer (C-LF), where relevant N is any nucleotide and collectively denotes a specific barcode sequence
ttccttgtctgattagttc

SEQ ID NO: 154 - DNA, 18 bp, Artificial Sequence
  Synthetic - Primer Binding Site in SARS CoV-2 Genome, where relevant N is any nucleotide and collectively denotes a specific barcode sequence
accttcgggaacgtggtt

SEQ ID NO: 155 - DNA, 18 bp, Artificial Sequence
  Synthetic - Primer Sequence (5′-3′), where relevant N is any nucleotide and collectively denotes a specific barcode sequence
accttcgggaacgtggtt

SEQ ID NO: 156 - DNA, 18 bp, Artificial Sequence
  Synthetic - primer (C-LB), where relevant N is any nucleotide and collectively denotes a specific barcode sequence
accttcgggaacgtggtt

SEQ ID NO: 157 - DNA, 23 bp, Artificial Sequence
  Synthetic - Primer Binding Site in SARS CoV-2 Genome, where relevant N is any nucleotide and collectively denotes a specific barcode sequence
tttgcccccagcgcttcagcgtt

SEQ ID NO: 158 - DNA, 23 bp, Artificial Sequence
  Synthetic - Primer Sequence (5′-3′), where relevant N is any nucleotide and collectively denotes a specific barcode sequence
aacgctgaagcgctgggggcaaa

SEQ ID NO: 159 - DNA, 56 bp, Artificial Sequence; identical to SEQ ID NO: 77
  Synthetic - primer (miseq C f wd 10), where relevant N is any nucleotide and collectively denotes a specific barcode sequence

SEQ ID NO: 160 - DNA, 23 bp, Artificial Sequence
  Synthetic - Primer Binding Site in SARS CoV-2 Genome, where relevant N is any nucleotide and collectively denotes a specific barcode sequence
cagacaaggaactgattacaaac

SEQ ID NO: 161 - DNA, 23 bp, Artificial Sequence
  Synthetic - Primer Sequence (5′-3′), where relevant N is any nucleotide and collectively denotes a specific barcode sequence
gtttgtaatcagttccttgtctg

SEQ ID NO: 162 - DNA, 56 bp, Artificial Sequence; identical to SEQ ID NO: 78
  Synthetic - primer (miseq C r ev 11), where relevant N is any nucleotide and collectively denotes a specific barcode sequence

SEQ ID NO: 163 - DNA, 51 bp, Artificial Sequence; identical to SEQ ID NO: 47
  Synthetic - barcoded FIP primer, N can be any nucletoide and collectively denotes a specific barcode sequence
  misc_feature (23)..(32): n is a, c, g, or t

SEQ ID NO: 164 - DNA, 40 bp, Artificial Sequence; identical to SEQ ID NO: 17
  Synthetic - BIP primer

SEQ ID NO: 165 - DNA, 19 bp, Artificial Sequence
  Synthetic - FIP primer F3
tggctactaccgaagagct

SEQ ID NO: 166 - DNA, 20 bp, Artificial Sequence
  Synthetic - FIP primer B3
tgcagcattgttagcaggat

SEQ ID NO: 167 - DNA, 25 bp, Artificial Sequence
  Synthetic - FIP primer LF
ggactgagatctttcattttaccgt

SEQ ID NO: 168 - DNA, 21 bp, Artificial Sequence
  Synthetic - FIP primer LB
actgagggagccttgaataca

SEQ ID NO: 169 - DNA, 56 bp, Artificial Sequence; identical to SEQ ID NO: 53
  Synthetic - FIP primer B_fwd_10

SEQ ID NO: 170 - DNA, 56 bp, Artificial Sequence; identical to SEQ ID NO: 54
  Synthetic - FIP primer B_rev_12

SEQ ID NO: 171 - DNA, 60 bp, Artificial Sequence
  Synthetic - PCR primer D501
aatgatacggcgaccaccgagatctacactatagcctacactctttccctacacgacgct

SEQ ID NO: 172 - DNA, 56 bp, Artificial Sequence
  Synthetic - PCR primer D701
caagcagaagacggcatacgagatcgagtaatgtgactggagttcagacgtgtgct

SEQ ID NO: 173 - DNA, 51 bp, Artificial Sequence; identical to SEQ ID NO: 73
  Synthetic - - barcoded C-FIP primers, N can be any nucleotide, collectively N′s denote a specific barcode sequence
  misc_feature (23)..(32): n is a, c, g, or t

SEQ ID NO: 174 - DNA, 39 bp, Artificial Sequence; identical to SEQ ID NO: 32
  Synthetic - primer C-BIP

SEQ ID NO: 175 - DNA, 18 bp, Artificial Sequence
  Synthetic - C-F3 primer
aacacaagctttcggcag

SEQ ID NO: 176 - DNA, 22 bp, Artificial Sequence
  Synthetic - C-B3 primer
gaaatttggatctttgtcatcc

SEQ ID NO: 177 - DNA, 19 bp, Artificial Sequence
  Synthetic - C-LF primer
ttccttgtctgattagttc

SEQ ID NO: 178 - DNA, 18 bp, Artificial Sequence
  Synthetic - C-LB primer
accttcgggaacgtggtt

SEQ ID NO: 179 - DNA, 56 bp, Artificial Sequence; identical to SEQ ID NO: 77
  Synthetic - PCR primer, PCR-C-forward primer

SEQ ID NO: 180 - DNA, 56 bp, Artificial Sequence; identical to SEQ ID NO: 78
  Synthetic - PCR primer, PCR-C-reverse primer

SEQ ID NO: 181 - DNA, 62 bp, Artificial Sequence; identical to SEQ ID NO: 55
  Synthetic - , pool-specific forward barcoding primer, N can be any nucleotide, where collectively N′s denotes a specific barcode sequence
  misc_feature (30)..(39): n is a, c, g, or t

SEQ ID NO: 182 - DNA, 58 bp, Artificial Sequence; identical to SEQ ID NO: 56
  Synthetic - pool-specific reverse barcoding primer, N can be any nucleotide, where collectively N′s denotes a specific barcode sequence
  misc_feature (25)..(34): n is a, c, g, or t

SEQ ID NO: 183 - DNA, 51 bp, Artificial Sequence; identical to SEQ ID NO: 73
  Synthetic - barcoded C-FIP primers, N can be any nucleotide, collectively N′s denote a specific barcode sequence
  misc_feature (23)..(32): n is a, c, g, or t

SEQ ID NO: 184 - DNA, 39 bp, Artificial Sequence; identical to SEQ ID NO: 32
  Synthetic - primer C-BIP

SEQ ID NO: 185 - DNA, 18 bp, Artificial Sequence
  Synthetic - C-F3 primer
aacacaagctttcggcag

SEQ ID NO: 186 - DNA, 22 bp, Artificial Sequence
  Synthetic - C-B3 primer
gaaatttggatctttgtcatcc

SEQ ID NO: 187 - DNA, 19 bp, Artificial Sequence
  Synthetic - C-LF primer
ttccttgtctgattagttc

SEQ ID NO: 188 - DNA, 18 bp, Artificial Sequence
  Synthetic - C-LB primer
accttcgggaacgtggtt

SEQ ID NO: 189 - DNA, 56 bp, Artificial Sequence; identical to SEQ ID NO: 77
  Synthetic - PCR primer, PCR-C-forward primer

SEQ ID NO: 190 - DNA, 56 bp, Artificial Sequence; identical to SEQ ID NO: 78
  Synthetic - PCR primer, PCR-C-reverse primer

SEQ ID NO: 191 - DNA, 62 bp, Artificial Sequence; identical to SEQ ID NO: 55
  Synthetic - pool-specific forward barcoding primer, N can be any nucleotide, where collectively N′s denotes a specific barcode sequence
  misc_feature (30)..(39): n is a, c, g, or t

SEQ ID NO: 192 - DNA, 58 bp, Artificial Sequence; identical to SEQ ID NO: 56
  Synthetic - pool-specific reverse barcoding primer, N can be any nucleotide, where collectively N′s denotes a specific barcode sequence
  misc_feature (25)..(34): n is a, c, g, or t

SEQ ID NO: 193 - DNA, 19 bp, Artificial Sequence
  Synthetic - RT-qPCR forward primer for N-gene
cacattggcacccgcaatc

SEQ ID NO: 194 - DNA, 20 bp, Artificial Sequence
  Synthetic - RT-qPCR reverse primer for N-gene
gaggaacgagaagaggcttg

SEQ ID NO: 195 - DNA, 25 bp, Artificial Sequence
  Synthetic - RT-qPCR probe sequence, 5′ FAM label, 3′ BBQ label
acttcctcaaggaacaacattgcca

SEQ ID NO: 196 - DNA, 16 bp, Artificial Sequence
  Synthetic - RT-qPCR forward primer MS2 gene
tgctcgcggatacccg

SEQ ID NO: 197 - DNA, 20 bp, Artificial Sequence
  Synthetic - RT-qPCR reverse primer MS2 gene
aacttgcgttctcgagcgat

SEQ ID NO: 198 - DNA, 24 bp, Artificial Sequence
  Synthetic - RT-qPCR probe MS2 gene, 5′ FAM label, 3′ BBQ label
acctcgggtttccgtcttgctcgt

(19) United States
(12) Patent Application Publication
(10) Pub. No.: US 2023/0151441 A1
(43) Pub. Date: May 18, 2023

(54) SEQUENCING-BASED POPULATION SCALE SCREENING

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Jonathan Schmid-Burgk, Cambridge, MA (US); David Li, Cambridge, MA (US)

(21) Appl. No.: 17/916,087

(22) PCT Filed: Apr. 2, 2021
(86) PCT No.: PCT/US2021/025532

§ 371 Date: Sep. 30, 2022

Related U.S. Application Data
(60) Provisional application No. 63/004,456, filed on Apr. 2, 2020.
(60) Provisional application No. 63/032,694, filed on May 31, 2020.

(51) Int. Cl. C12Q 1/70; C12Q 1/6806; C12Q 1/6844; C12Q 1/6869

(52) U.S. Cl. CPC C12Q 1/701; C12Q 1/6806; C12Q 1/6844; C12Q 1/6869; C12Q 2600/16

(57) ABSTRACT

Provided herein are methods and kits for parallel detection one or more target sequences across multiple samples, comprising separating a set of samples into one or more pooled sets, wherein each sample may comprise an initial amplicon comprising one or more target sequences and at least one barcode; conducting an amplification reaction on the one or more pooled sets to further amplify the amplicons, and optionally further adding an additional barcode to the amplicon; sequencing the amplicons; identifying individual samples from the pooled sample set that are positive for the one or more target sequences based on sequencing of the amplicons, wherein identification is based, at least in part, on detection of the unique combination of barcodes.

### CROSS-REFERENCE TO RELATED APPLICATIONS

This application claims the benefit of U.S. Provisional Application No. 63/004,456, filed Apr. 2, 2020 and U.S. Provisional Application No. 63/032,694, filed May 31, 2020. The entire contents of the above-identified applications are hereby fully incorporated herein by reference.

### SEQUENCE LISTING

This application contains a sequence listing filed in electronic form as an ASCII.txt file entitled BROD-5130WP_ST25.txt, created on Apr. 2, 2021 and having a size of 55,672 bytes. The content of the sequence listing is incorporated herein in its entirety.

### TECHNICAL FIELD

The subject matter disclosed herein is generally directed to detection of viral RNA in clinical samples.

### BACKGROUND

The recent global spread of a novel coronavirus, SARS-CoV-2 has already resulted in more than half a million confirmed cases and 23,000 deaths since its emergence several months ago. Early epidemiological studies indicate that the exponential spread of COVID-19 (the disease caused by SARS-CoV-2) can be slowed by restrictive isolation measures, but these draconian measures place an enormous burden on societies and economies. Moreover, once isolation measures are lifted, exponential spread is predicted to resume. To stop this catastrophic cycle, we need population-scale testing that can enable rapid identification of potential outbreak areas where local isolation regulations can be temporarily enacted.

Currently, COVID-19 testing is performed mainly using RT-qPCR to amplify and detect one of several highly conserved regions of the SARS-CoV2 genome, or by detecting serum antibodies specific for viral proteins. The global capacity for testing, however, has been limited by a combination of access and supply issues. As such, there exists an urgent need for improved testing strategies for infectious organisms, such as SARS-CoV2, that are adaptable, for example, to scalable deployment.

### SUMMARY

Described in several exemplary embodiments herein are kits comprising (a) amplification reagents and (b) one or more primer sets comprising two or more primers, wherein at least one primer of at least one of the one or more primer sets comprises one or more barcodes, and wherein the primer sets are configured to amplify one or more target sequences from a sample in one or more amplification steps to generate amplicons that comprise the one or more target sequences and a unique combination of barcodes.

In certain example embodiments, at least one of the one or more primer sets is configured for loop-mediated isothermal amplification (LAMP) or reverse transcription loop-mediated isothermal amplification (RT-LAMP) and comprises at least one forward inner primer (FIP), backward inner primer (BIP), or both.

In certain example embodiments, one, two, or more primers of the at least one or more primer sets comprises one or more barcodes.

In certain example embodiments, the barcode(s) is/are inserted between the two target-specific sequences of the FIP, the BIP, or both.

In certain example embodiments, one or more of the one or more primer sets is configured for PCR amplification and wherein one or more primers of the one or more primer sets configured for PCR amplification comprises one or more PCR barcodes, sequencing adaptors, or both.

In certain example embodiments, the individual barcodes are derived from a defined set of barcodes and configured such that individual barcodes are capable of being used in more than one kit and configured such that each kit receives a unique combination of barcodes.

In certain example embodiments, the barcodes in the defined set of barcodes are selected to avoid barcodes having a sequence portion that is the reverse complement to the 3′ end of a forward inner primer (FIP) or a backward inner primer (BIP).

In certain example embodiments, the number of individual barcodes used per kit is determined, at least in part, on a total number of barcode sequences in the defined set of barcodes, and a number of samples to be processed in parallel.

In certain example embodiments, the number of barcodes is between 2 and 20.

In certain example embodiments, the number of samples to be processed in parallel is optimized based on an expected or empirically determined fraction of positive samples, an estimated or empirically determined fraction of ineffective barcodes, a frequency of sample barcode dropout, a heterogeneity of sample representation in sequencing data, a false-positive cutoff rate, a false-negative cutoff rate, or a combination thereof.

In certain example embodiments, at least two of the primers of at least one of the one or more primer sets are barcoded.

In certain example embodiments, each barcode is between 4 and 40 bases in length.

In certain example embodiments, each barcode is between 8 and 15 based in length.

In certain example embodiments, the barcoded primers are included in the kit at equal concentrations.

In certain example embodiments, the amplification reagents are isothermal amplification reagents, polymerase chain reaction reagents, or both.

In certain example embodiments, the isothermal amplification reagents are loop-mediated isothermal amplification (LAMP) or reverse transcription loop-mediated isothermal amplification (RT-LAMP) reagents.

In certain example embodiments, the kit further comprises a lysis reagent.

In certain example embodiments, the kit further comprises a control template DNA or RNA sequence.

In certain example embodiments, the kit further comprises a sample collection component.

In certain example embodiments, the sample collection component is configured for collection of a nasal swab, an oral swab, a nasal wash, an oral wash, a fecal sample, a wound swab, or a combination thereof.

In certain example embodiments, the kit further comprises a sample dosing component.

In certain example embodiments, the kit further comprises a reaction vessel comprising a pre-mixed combination of amplification reagents and barcoded primers and configured to be sealed after receiving the sample, sample collection component, sample dosing component, or a combination thereof.

In certain example embodiments, the reaction vessel is configured for use in an isothermal amplification reaction conducted at a point of care.

In certain example embodiments, the kit further comprises one or more heating components wherein the one or more heating components are configured for use in an isothermal reaction conducted at a temperature between 45° C. to 75° C.

In certain example embodiments, the one or more chemical heating reaction reagents are configured for use in an isothermal reaction conducted at a temperature between 55° C. to 70° C.

In certain example embodiments, the one or more heating components are configured for use in an isothermal reaction conducted at a temperature between 60° C. to 65° C.

In certain example embodiments, the one or more heating components is or includes one or more chemical heating reagents.

In certain example embodiments, the one or more chemical heating reagents comprises molten sodium acetate.

In certain example embodiments, the one or more target sequences is used to genotype a subject, to detect a disease marker, detect an infectious agent, or a combination thereof.

In certain example embodiments, the infectious agent is a viral agent.

In certain example embodiments, the viral agent is an RNA virus.

In certain example embodiments, the RNA virus is a coronavirus.

In certain example embodiments, the coronavirus is SARS-CoV2.

In certain example embodiments, the SARS-CoV2 is a SARS-CoV2 variant selected from B.1.1.7, B.1.351, P.1, or a combination thereof. In some embodiments the SARS-COV2 variant comprises a D614G mutation.

In certain example embodiments, the kit further comprises control primers that are configured to amplify a target sequence of endogenous RNA of the sample to confirm successful sample collection.

In certain example embodiments, the kit further comprises a colorimetric or turbidimetric indicator.

Described in certain example embodiments herein are methods of parallel detection of one or more target sequences across multiple samples, comprising
a. separating a set of samples into one or more pooled sample sets, wherein each sample comprises an initial amplicon comprising one or more target sequences and at least one barcode;
b. conducting an amplification reaction on the one or more pooled sample sets to further amplify the amplicons, and optionally further adding an additional barcode to the amplicon;
c. sequencing the amplicons; and
d. identifying individual samples from the pooled sample set that are positive for the one or more target sequences based on sequencing of the amplicons, wherein identification is based, at least in part, on detection of the unique combination of barcodes.

In certain example embodiments, the amplicons in each individual sample comprising the set of samples of step (a) are generated by conducting an isothermal amplification reaction on each individual sample using one or more primer sets and wherein a primer in each primer set comprises a barcode and each set of primers comprises a combination of barcodes unique to each sample.

In certain example embodiments, the number of barcodes used per sample is determined, at least in part, on the total number of barcode sequences in a defined set of barcode sequences and a number of samples to be processed in parallel.

In certain example embodiments, the number of barcodes used per sample is between 2 and 20.

In certain example embodiments, barcodes are selected so as to avoid barcodes comprising a sequence portion that is a reverse complement to the 3′ end of a primer, in particular the 3′ end of a forward inner primer (FIP).

In certain example embodiments, the number of samples to be processed in a pooled set is optimized based on an expected or empirically determined fraction of positive samples, an estimated or empirically determined fraction of ineffective barcodes, a frequency of sample barcode dropout, a heterogeneity of sample representation in sequencing data, a false-positive cutoff rate, a false-negative cutoff rate, or a combination thereof.

In certain example embodiments, the number of pooled sets is 1-11, 12-96, or 384.

In certain example embodiments, the isothermal amplification reaction is loop-mediated isothermal amplification (LAMP) or reverse transcription loop-mediated isothermal amplification (RT-LAMP).

In certain example embodiments, the barcode sequences are inserted between two target-specific sequences of either a forward inner primer (FIP), a backward inner primer (BIP), or both.

In certain example embodiments, the samples are further heat-inactivated either prior to or after being pooled into a pooled sample set.

In certain example embodiments, the amplicon resulting from amplification of the pooled sets spans a fraction of the target nucleic acid sequence not covered or only partially covered by the primers used to generate an initial amplicon.

In certain example embodiments, the amplicon resulting from the amplification of pooled sets spans one or both of the junctions between a barcode sequence and the target nucleic acid sequence.

In certain example embodiments, sequencing the amplicons comprises deep sequencing of the amplicons.

In certain example embodiments, the set of samples is diluted to between 1:1,000 to 1:1,000,000 prior to the amplification reaction of (b).

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1H—LAMP-Seq: Scalable deep-sequencing based approach for SARS-CoV-2 detection. (FIG. 1A) Schematic outline of a proposed scalable testing procedure involving remote lysis and inactivation of virus samples, and centralized barcoded RT-LAMP, pooling, and sequencing. (FIG. 1B) Schematic outline of a proposed scalable testing procedure involving remote barcoded RT-LAMP and sample pooling, and centralized sequencing. (FIG. 1C) Schematic of anticipated enzymatic reactions and reaction products. (FIG. 1D) Experimental validation of barcode insertion into FIP primers employed in LAMP reactions. All steps were performed as described in the Methods section, with the exception that plasmid DNA containing the SARS-CoV-2 N-gene (IDT) was used as template instead of a swab sample, no Bst 3.0 or Tris buffer were added, and the reaction was scaled down to a volume of 25 μl. Samples were run on a 1% agarose gel and visualized using ethidium bromide. (FIG. 1E) Barcoded LAMP reactions templated with either 100 or 10,000 dsDNA molecules were combined after heat inactivation as described for FIG. 1D. Reactions were PCR amplified and sequenced on an Illumina MiSeq sequencer. Relative read counts are shown as mean and standard deviation from two experimental replicates. (FIG. 1F) (SEQ ID NO: 1) RT-LAMP reactions with a combination of three barcoded FIP primers, but without Tris or Bst 3.0 were templated with synthetic RNA, and were sequenced using a MiSeq sequencer. Base frequencies are depicted by the size of each letter without applying any read filtering. (FIG. 1G) Sensitivity measurement of RT-LAMP reactions as described for FIG. 1D templated with indicated numbers of synthetic RNA molecules. After PCR amplifying the products, positive reactions were counted using a 1% agarose gel. (FIG. 111) Likelihood function of the probability of detection for a single RNA molecule.

FIG. 2—Scenarios of scalable deployment of deep-sequencing based SARS-CoV-2 detection. Proposed deployment scenario 1 for testing the German population in 3-6 months using centralized swab processing (top sequence). Alternative deployment scenario 2 involving home processing, random pooling, and rolling sequencing (bottom sequence).

FIG. 3—Graphs that show k_{OPT} and FPP_{OPT} as % Infected varies with m=10⁴ and b=10⁵.

FIG. 4—A graph showing FPP as % Infected varies with m=10⁴ and b=10⁵ and k=2, 3, 4 or 5.

FIG. 5—A graph showing maximum compression ratio as the % infected varies for m=10⁴ and b=10⁵.

FIG. 6—Graphs showing False Negative Probability (FNP_{Δ}) as the % infected varies form 10⁴ 10⁴ and b=10⁵.

FIG. 7—Graphs showing FNP_{Δ}, FPP_{Δ}, FNP_{Δ}), FPP_{Δ2}, as the % infected varies for m=10⁴ and b=10⁵.

FIG. 8—Graphs showing error rates for various m₂ as the % infected varies for m 10⁴ and b=10⁵.

FIG. 9—Graphs showing error rates for various I'm as the % infected varies for m=10⁴ and b=10⁵.

FIG. 10—Graphs showing error rates for various m₂ as the % infected varies for m=10⁴ and b=10⁵.

FIG. 11—Shows nucleotide sequences for amplicon A (SEQ ID NOs: 2-12).

FIG. 12—Shows nucleotide sequences for amplicon B (SEQ ID NOs: 13-27).

FIG. 13—Shows nucleotide sequences for amplicon C (SEQ ID NOs: 28-38).

FIG. 14—Graphs showing error rates for various m₂ as the % infected varies for m=384 and b=10⁵.

FIG. 15— Graphs showing error rates for various m₂ as the % infected varies for m=10³ and b=10⁵.

FIG. 16—Experimental validation of barcoded LAMP protocol. All steps were performed as described in the Suggested Protocol until after the first PCR, with the exception that plasmid DNA containing the SARS-CoV-2 N-gene (IDT) was used as template instead of a swab sample, 1 ng/μl pX330 plasmid DNA was present as unspecific decoy DNA, and WarmStart LAMP 2× Master Mix (NEB) was used instead of buffer, MgSO₄, dNTPs, Triton X-100, and polymerase. Samples were run on an 1% agarose gel and visualized using Ethidium bromide.

FIG. 17—Shows a proposed choice of academic sequencing facilities in Germany with necessary equipment and cost estimation of the first deployment scenario.

FIGS. 18A-18C—Simulation of False Positive Probability and False Negative Probability depending on m₂=the number of sub-pools (FIG. 18A), m=the complexity of the barcode library (FIG. 18B), and k=the number of barcodes per sample (FIG. 18C) when utilizing a compressed barcode space for the second deployment scenario.

FIG. 19A-19F—Clinical validation and optimization of LAMP-Seq. (FIG. 19A) Outline of the protocol employed for validating LAMP-Seq (bottom row) against an established clinical RT-qPCR pipeline (top row). (FIG. 19B) LAMP-Seq read numbers obtained per sample in comparison to RT-qPCR Ct values indicated in bottom row. The red dashed line indicates a threshold of 100,000 reads. (FIG. 19C) Summary statistics of validation experiments detailed in FIGS. 19A-19B. (FIG. 19D) (SEQ ID NO: 39) NextSeq data obtained from a SARS-CoV-2-positive swab sample employing LAMP-Seq. Base frequencies are depicted by the size of each letter without applying any read filtering. (FIG. 19E) Quantitative assessment of barcode swapping during LAMP-Seq and its dependence on pre-dilution of pooled RT-LAMP reactions before PCR (left panel, 1,000-fold, right panel, 1,000,000-fold). LAMP-Seq was performed as described in the Methods section, with the exception that synthetic RNA was used as template instead of a swab sample, no Bst 3.0 or Tris buffer were added, and the reactions were scaled down to a volume of 25 μl. Numbers in the plot indicate read numbers for non-templated negative control reactions. (FIG. 19F) (SEQ ID NOs: 40-45) Empirical performance assessment of 480 random LAMP-Seq barcode primers. 480 barcoded FIP primers were mixed at equimolar concentration and were used as a pool in four replicate LAMP-Seq reactions templated by synthetic RNA. Raw sequencing data were analyzed using LAMP-Seq-Inspector v1.0 (http://manuscript.lamp-seq.org/Inspector.htm). Read counts are shown for barcodes in descending order, with the worst-performing 5% of all barcode sequences highlighted in light red and the worst-performing barcode sequences listed.

FIGS. 20A-20G—Modeling of compressed barcoding schemes for LAMP-Seq, enabling population-scale testing. (FIG. 20A) Schematic illustration of an uncompressed and a compressed single barcode scheme. (FIGS. 20B-20C) Calculated False Positive Probability and False Negative Probability depending on the global positive frequency of samples (FIG. 20A) and m=the complexity of the barcode library (FIG. 20B) for various numbers of pools per run, utilizing a compressed single barcode space accounting for barcode loss. Dashed grey lines indicate a probability threshold of 0.2%. (FIG. 20D) Schematic of anticipated enzymatic reactions and reaction products for dual barcoding. The depicted positioning of barcodes 4-6 in LAMP products is not the only conformation, with an alternative orientation indicated by dotted circles. (FIG. 20E) Schematic illustration of an uncompressed and a compressed dual barcode scheme. (FIGS. 20F-20G) Numerical simulation of False Positive Probability and False Negative Probability depending on the global positive frequency of samples, m₁=the number of FIP barcodes, m₂=the number of BIP barcodes, the number of pools per run, and k₁₂′=the number of required barcode pairs per positive sample, over 100 iterations. Dashed grey lines indicate a probability threshold of 0.2%.

FIG. 21—Template switching for Scenario 3 (Example 3) with m₁=m₂=5, k₁=k₂=1, Δ_{switch}=0.02, and no barcode loss. The probability of inferring a particular sample as positive is shown next to each patient sample.

FIG. 22—Template switching for Scenario 5 (Example 8) with m₁=m₂=5, k₁=k₂=1, Δ_{switch}=0.02, k′₁₂=4, and no barcode loss. The probability of inferring a particular sample as positive is shown next to each patient sample.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Currently, COVID-19 testing is performed mainly using RT-qPCR to amplify and detect one of several highly conserved regions of the SARS-CoV2 genome, or by detecting serum antibodies specific for viral proteins. The global capacity for testing, however, has been limited by a combination of access and supply issues. As such there is an immediate need for improved methods of testing for infectious organisms, such as SARS-CoV2 that can allow for scalable deployment.

Provided in several embodiments herein are methods that can allow for a population-scale detection method based on massively parallel RT-LAMP. In some embodiments, the method includes a single heat-step for each individual sample (e.g., nasal swab, nasal wash, or potentially saliva), followed by pooled processing, parallelizable deep sequencing, and computational analysis. By employing compressed sensing via use of unique barcode combinations per sample, the cost and complexity of testing can be significantly reduced. The cost is estimated to be less than five USD and this approach would be scalable to millions of samples per day.

Embodiments disclosed herein provide systems and methods that allow for massively parallel population-scale screening with reduced cost and logistical overhead. Applications include, for example, genotyping, screening for disease markers, and infectious disease testing and monitoring. In certain embodiments kits and methods are designed to allow for pooled processing, parallelizable deep sequencing, and computational analysis. By employing compressed sensing via use of unique barcode and barcode combinations per sample, the cost and complexity of testing can be significantly reduced.

### Kits

Described in several embodiments herein are kits that provide reagents for initial processing of samples and for use with the methods disclosed in detail below. As noted above, the method allows for massively parallel processing of multiple samples that are pooled together. The ability to identify positive samples from the pooled group of samples relates to incorporation of unique barcode combinations into amplicons generated from an initial amplification reaction on individual samples that are then further analyzed according to the methods described herein. Thus, the kits described herein provide an embodiment designed to generate these initial amplicons from individual samples and incorporate the unique combination of barcode information into the amplicon.

In some exemplary embodiments herein, the kits comprise amplification reagents and a set of primers. In certain example embodiments, a kit further comprises a lysis reagent. In certain example embodiments, the kit comprises a sample collection component. In certain example embodiments, the kit may comprise a reaction vessel comprising a pre-mixed combination of amplification reagents and barcoded primer sets. In certain example embodiments the reaction vessel is designed to receive a sample or the sample collection component and be sealed. The initial amplification reaction on the individual sample can be done at the point-of-care or the reaction vessel containing the sample and sample collection component can be sent to a central processing facility for pooling and parallel processing according to the methods disclosed herein. In certain examples, embodiment the kit further comprises a control sequence that can be amplified by the primers in the kit. The control may be included in the pre-mixed solution with the amplification reagents and the primer sets.

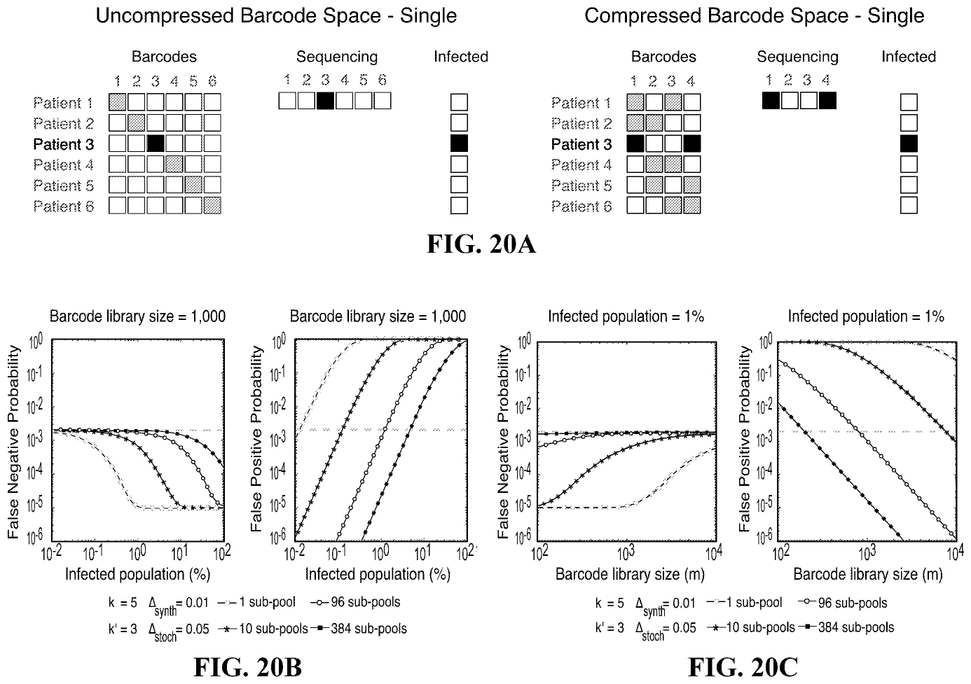

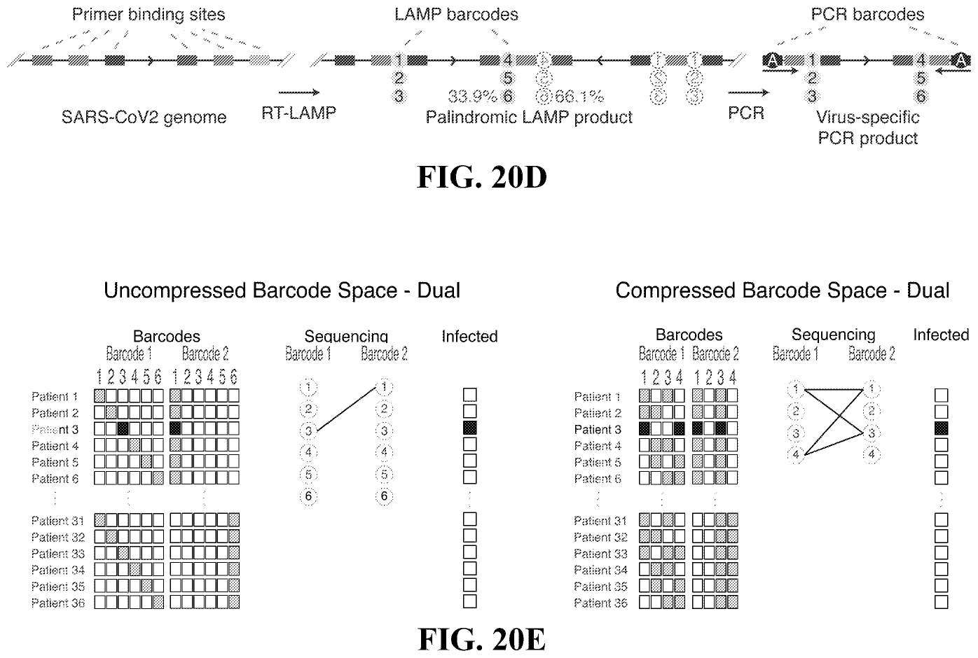

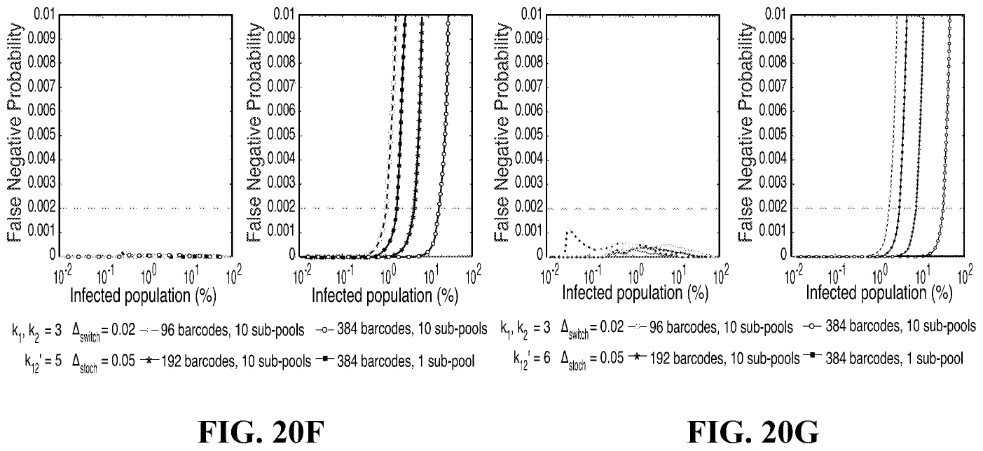

In some embodiments, a set of kits may be prepared, each kit comprising a set of primers. A set of kits may be designed according to a desired set of samples to be analyzed by pooling, and thus a number of barcodes per sample and a number of barcode primers. In an example embodiment, and described in the examples, 100,000 patient samples could be uniquely barcoded using 100 FIP primers, 100 BIP primers for a set of kits designed for LAMP amplification. With compression, over 20 billion samples can each be assigned a unique combination of barcodes using a combination of 3 FIP primers and a combination of 3 BIP primers per patient sample from a pool of 96 barcoded FIP and 96 barcoded BIP primers, see e.g., FIG. 20E.

### Primers

In some embodiments, the kit includes one or more primer sets where each primer set includes two or more primers. In some embodiments, two primers of the two or more primers of a primer set forms a primer pair. A primer pair refers to primers that are each capable of hybridizing to different sequences of a nucleic acid and are designed and configured to together define the region of the nucleic acid (e.g., DNA) that is amplified during an amplification reaction, such as a PCR or PCR-based amplification reaction. Any given primer set can have one, two, or more primer pairs. For example, a primer set having 6 primers, can include 1, 2, 3, or more primer pairs. It will be appreciated that a first primer can form a primer pair with more than one second primers. For example, a forward primer can form a primer pair with multiple different reverse primers. The primer pairs in primer sets having multiple primer pairs can be configured to amplify different and/or overlapping nucleic acid regions.

In some embodiments, the kit includes one or more primer sets including two or more primers, where at least one primer of at least one of the one or more primer sets includes one or more barcodes, and where the one or more primer sets are configured to amplify one or more target sequences in one or more amplification steps from a sample in to generate amplicons that comprise the one or more target sequences and a unique combination of barcodes. In some embodiments, the barcoded primers present in the kit are included in the kit at equal amounts or concentrations. In some embodiments, each kit, each primer set, and/or each have a unique barcode or set of barcodes. In some embodiments, two or more barcodes in a kit, each primer set, and/or one or more primers are different from each other. In some embodiments, two or more barcodes in a kit, each primer set, and/or one or more primers are the same from other. In some embodiments, all primers and/or primer sets in a kit contain a barcode unique to that kit. In some embodiments, each primer set contains a barcode that is unique to each primer set. In some embodiments, each primer pair contains a barcode unique to that primer pair. In some embodiments, each primer contains a barcode unique to that primer.

In some embodiments, the kit includes at least one primer set that is configured for loop-mediated isothermal amplification (LAMP) or reverse transcription loop-mediated isothermal amplification (RT-LAMP) and comprises at least one forward inner primer (FIP), backward inner primer (BIP), or both. In some embodiments, one, two, or more primers in a primer set includes one or more barcodes. In some embodiments, the barcode(s) is/are inserted between the two target-specific sequences of either the FIP, the BIP, or both.

In some embodiments, the kit contains one or more barcodes, such as in one or more primers of a primer set, that is/are unique to a first amplification and one or more barcodes, such as in one or more primers of a primer set that are unique to a second, third, fourth, fifth, etc. amplification reaction.

In some embodiments, the kit can include one or more primer sets that are configured for a PCR amplification, wherein one or more primers of the one or more primer sets configured for PCR amplification comprises one or more PCR barcodes, sequencing adaptors, or both. In some embodiments, the one or more primer sets configured for PCR amplification contains a barcode unique to the PCR amplification primer sets and/or sequencing adaptors.

In some embodiments, one, two, or more primers of the at least one or more primer sets includes one or more barcodes. In some embodiments, the barcode(s) is/are inserted between the two target-specific sequences of the FIP, the BIP, or both.

In some embodiments, the kit includes one or more primer sets configured for non-LAMP amplification by PCR. In some of these embodiments one or more primers of these one or more primer sets comprises one or more PCR barcodes, sequencing adaptors, or both.

In some embodiments, a set of primers included in the kit comprises two or more primer pairs and each primer pair comprises a different barcode. In some embodiments, a single primer in the primer pair can have a barcode. In some embodiments, both primers in a primer pair can have a barcode, where the barcodes in each of the primers of the primer pair are the same or different. Each primer pair may amplify different or overlapping target sequences of a target polynucleotide. Example target polynucleotides are discussed in further detail below. Kits may be configured to detect a single target polynucleotide or more than one target polynucleotide so long as multiple primer sets are used for each target polynucleotide to incorporate a unique combination of barcodes into the resulting amplicons.

The number of barcoded primers to be included in a given kit is determined, in part, by the size of the barcode set for which individual barcodes can be pulled from and the number of samples that are to be pooled and run together. Guidance on the size of the barcode set and size of the number of samples to be pooled together is discussed in further detail below and in the Examples section. In certain example embodiment, the number of barcodes incorporated using the aforementioned primers pairs is 2, 3, 4, 5, 6, 7, 8, 9, 10, 11, 12, 13, 14, 15, 16, 17, 18, 19, or 20.

Primer design is well known to those of skill in the art. In this regard, reference is made to Shi et al. Chin Sci Bull 48(12):1165-1169 (2003); Chen et al. Analyst 145(2):440-444 (2020); Higgins et al. Bioinformatics 35(4):682-684 (2019); Liu et al. World J Microbiol Biotechnol 35(6):95 (2019); Jia et al. Front Microbiol 10:2860 (2019); Hardinge et al. Sci Rep 9(1):7400 (2019); Morabito et al. Mol Diagn Ther 17(3):185-192 (2013); de Baar et al. J Clin Microbiol 37(6):1813-1818 (1999); Lopez-Jimena et al. PLoS Negl Trop Dis 12(5):e0006381 (2018). The primers and barcodes may be further designed such that the do not form strong secondary structures or where the barcode has a GC content within a specified range.

### Barcodes

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more).

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

In certain example embodiments, barcodes are designed to avoid barcodes with sequence portions that are the reverse complement of a 3 prime end of a primer, such as the 3′ end of a HP primer or a BIP primer used in isothermal amplifications embodiments disclosed herein.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associated with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type (SEQ ID NO: 46).

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2nd Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequenceable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, SynBio software system, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17;106(7):2289-94).

### Amplification Reagents

In some embodiments, the kit includes one or more amplification reagents. Amplification reagents and systems known in the art can be designed for use with the methods and systems detailed herein. In some embodiments, amplification is isothermal. Any suitable RNA or DNA amplification technique may be used. In certain example embodiments, the RNA or DNA amplification is an isothermal amplification. In certain example embodiments, the isothermal amplification may be nucleic-acid sequence-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In particular embodiments a first amplification method is utilized, for example an isothermal amplification, and a second amplification method is utilized subsequently, for example, a non-isothermal amplification method. In certain embodiments when two amplification methods are used, the amplification methods are the same, in other embodiments, the amplification methods are different. In an aspect, at least one of the amplification methods is an isothermal amplification that retains one or more barcodes in the amplicon. In an aspect, at least one of the amplification methods comprise amplifying a plurality of pooled samples. The isothermal amplification kits may be designed, via the selection of appropriate polymerases and buffers, to work over a wide range of temperatures.

### Loop-Mediated Isothermal Amplification

In certain example embodiments, a loop-mediated isothermal amplification (LAMP) reaction may be used to target nucleic acids, which encompasses both LAMP and RT-LAMP reactions. LAMP can be performed with a four-primer system for isothermal nucleic acid amplification in conjunction with a polymerase. Notomi et al., Nucleic Acids Res. 2000, 28, 12, Nagamine et al., Molecular and Cellular Probes (2002) 16, 223-229, doi: 10.1006/mcpr.2002.0415. When performing LAMP with a 4-primer system, two loop-forming inner primers, denoted as FIP and BIP, are provided with two outer primers, F3 and B3. The inner primers each contain two distinct sequences, one for priming in the first stage of the amplification and the other sequence for self-priming in subsequent amplification states. The two outer primers initiate strand displacement of nucleic acid strands initiated from the FIP and BIP primers, thereby generating formation of loops and strand displacement nucleic acid synthesis utilizing the provided polymerase. LAMP can be conducted with two to six primers, ranging from only the two loop-forming primers, up to at least the addition of 2 additional primers, LF and LB along with the two outer primers and two inner primers. LAMP technologies advantageously have high specificity and can work at a variety of pH and temperature. In a preferred aspect, the LAMP is an isothermal reaction at between about 45° C. to 75° C., 55 to 70° C. or 60° C. to 65° C. Colorimetric LAMP (Y. Zhang et al., doi:10.1101/2020.92.26.20028373), RT-LAMP (Lamb et al., doi: 10.1101/2020.02.19.20025155; and Yang et al., doi:10.1101/2020.03.02.20030130) have been developed for detection of COVID-19, and are incorporated herein by reference in their entirety.

In certain embodiment, the LAMP reagents may include Bst 2.0+RTx or Bst 3.0 from New England Biolabs.

In an aspect, the primer sets for LAMP comprise a unique combination of barcodes and are designed to amplify one or more target sequences, generating amplicons that comprise the one or more target sequences along with the unique combination of barcodes. The barcodes can be designed as described elsewhere herein, and may comprise a defined set that provides a unique combination of barcodes for each sample. In an aspect, the barcode sequences are inserted between two target specific sequences of the FIP primer, the BIP primer, or both. In an aspect, a unique set of barcoded FIP (or BIP) primers are utilized in the LAMP reaction. The number of unique barcoded FIP (or BIP) primers in a set will vary based on assumptions for the assay to be conducted, for example, number of samples per run, depth of sequencing reads. One or more FIP or BIP primers may comprise a barcode in the set. In an aspect, the unique set of barcoded FIP (or BIP) primers comprise 2, 3, 4, 5, 6, 7, 8, 9, 10, 11, 12, 13, 14, 15 or more FIP (or BIP) sequences, in which one or more of the sequences comprise a unique barcode. Use of the FIP (or BIP) primers can provide a resultant amplicon that preferably spans a fraction of the target nucleic acid sequence that is not covered by the amplification primers. In an aspect, the amplicon spans one or both junctions between a barcode sequence and the target nucleic acid sequence. In particular embodiments, the unique set of FIP (or BIP) primers can be utilized with the BIP (or FIP) primer, F3 primer, B3 primer, LF primer and LB primer. During the LAMP reaction, the barcode is inserted into the loop region of the LAMP product. Upon heating of the sample to a temperature sufficient for LAMP amplification, e.g., 50° C.-72° C. (e.g., 50, 51, 52, 53, 54, 55, 56, 57, 58, 59, 60, 61, 62, 63, 64, 65, 66, 67, 68, 69, 70, 71, to/or 72° C.), using a polymerase and, optionally a reverse transcriptase (in the event RT-LAMP is utilized). Preferably the enzymes utilized in the LAMP reaction are heat-stabilized. One or more of the barcodes can be associated with the source of the sample, e.g., a patient identification, origin-specific barcode. Optionally, a control template is further provided with the sample, which may differ from the target sequence but share primer binding sites. In an aspect, the sample can continue to be heated subsequent to time sufficient to complete the LAMP reaction, to about 90° C. to about 100° C. (such as about 90, 91, 92, 93, 94, 95, 96, 97, 98, 99, to/or 100° C.), inactivating the enzymes and/or sterilizing the sample. The sample can be further processed through additional reactions, with or without a purification step. In an exemplary embodiment, a plurality of samples are pooled and subjected to one or more PCR reactions, utilizing the unique barcode(s) of the LAMP amplicons to identify the source of the sample. In an aspect, 10,000 to 100,000, or more samples can be pooled. The samples can be optionally tagmented by Tn5 with two handles prior to PCR reactions, or two PCR primers with varying Illumina handles can be used that share the same binding sequence to the viral sequence. Deep sequencing via an Illumina NextSeq sequencer can be performed, identifying positive samples comprising the presence of target sequences, and associating positive samples with a patient-source.

### Recombinase Polymerization Amplification

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. (e.g., 37, 38, 39, 40, 41, to/or 42° C.). The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

### NASBA

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected thus further enhancing the sensitivity of the assay. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. (e.g., 37, 38, 39, 40, 41, to/or 42° C.). The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

### Transposase Based Amplification

Embodiments disclosed herein provide systems and methods for isothermal amplification of target nucleic acid sequences by contacting oligonucleotides containing the target nucleic acid sequence with a transposon complex. The oligonucleotides may be single stranded or double stranded RNA, DNA, or RNA/DNA hybrid oligonucleotides. The transposon complex comprises a transposase and a transposon sequence comprising one or more RNA polymerase promoters. The transposase facilitates insertion of the one or more RNA polymerase promoters into the oligonucleotide. A RNA polymerase promoter can then transcribe the target nucleic acid sequence from the inserted one or more RNA polymerase promoters. One advantage of this system is that there is no need to heat or melt double-stranded DNA templates, since RNA polymerase polymerases require a double-stranded template. Such isothermal amplification is fast and simple, obviating the need for complicated and expensive instrumentation for denaturation and cooling. In certain example embodiments, the RNA polymerase promoter is a native of modified T7 RNA promoter.

The term “transposon”, as used herein, refers to a nucleic acid segment, which is recognized by a transposase or an integrase enzyme and which is an essential component of a functional nucleic acid-protein complex (i.e., a transposome) capable of transposition. The term “transposase” as used herein refers to an enzyme, which is a component of a functional nucleic acid-protein complex capable of transposition and which is mediating transposition. The term “transposase” also refers to integrases from retrotransposons or of retroviral origin. Transposon complexes form between a transposase enzyme and a fragment of double stranded DNA that contains a specific binding sequence for the enzyme, termed “transposon end”. The sequence of the transposon binding site can be modified with other bases, at certain positions, without affecting the ability for transposon complex to form a stable structure that can efficiently transpose into target DNA.

In embodiments provided herein, the transposon complex may comprise a transposase and a transposon sequence comprising one or more RNA polymerase promoters. The term “promoter” refers to a region of DNA involved in binding the RNA polymerase to initiate transcription. In specific embodiments, the RNA polymerase promoter may be a T7 RNA polymerase promoter. The T7 RNA promoter may be inserted into the double-stranded polynucleotide using the transposase. In some embodiments, insertion of the T7 RNA polymerase promoter into the oligonucleotide may be random.

The frequency of transposition is very low for most transposons, which use complex mechanisms to limit activity. Tn5 transposase, for example, utilizes a DNA binding sequence that is suboptimal and the C-terminus of the transposase interferes with DNA binding. Mechanisms involved in Tn5 transposition have been carefully characterized by Reznikoff and colleagues. Tn5 transposes by a cut-and-paste mechanism. The transposon has two pairs of 19 bp elements that are utilized by the transposase: outside elements (OE) and inside elements (IE). One transposase monomer binds to each of the two elements that are utilized. After a monomer is bound to each end of the transposon, the two monomers dimerize, forming a synapse. Vectors with donor backbones of at least 200 bp, but less than 1000 bp, are most functional for transposition in bacteria. Transposon cleavage occurs by trans catalysis and only when monomers bound to each DNA end are in a synaptic complex. Tn5 transposes with a relaxed target site selection and can therefore insert into target DNA with little to no target sequence specificity.

The natural downregulation of Tn5 transposition can be overcome by selection of a hyperactive transposase and by optimizing the transposase-binding elements (York et al. 1998. Nucleic Acid Res. 26(8): 1927-1933). A mosaic element (ME), made by modification of three bases of the wild type OE, led to a 50-fold increase in transposition events in bacteria as well as cell-free systems. The combined effect of the optimized ME and hyperactive mutant transposase is estimated to result in a 100-fold increase in transposition activity. Goryshin et al showed that pre-formed Tn5 transposition complexes could be functionally introduced into bacterial or yeast by electroporation (Goryshin et al. 2000. Nat. Biotechnol. 18(1): 97-100). Linearization of the DNA, to have inverted repeats precisely positioned at both ends of the transposon, allowed Goryshin and coworkers to bypass the cutting step of transposition thus enhancing transposition efficiency.

In some embodiments, the transposase may be used to tagment the oligonucleotide sequence comprising the target sequence. The term “tagmentation” refers to a step in the Assay for Transposase Accessible Chromatin using sequencing (ATAC-seq) as described. (See, Buenrostro, J. D., et al., 2013. Nat. Methods. 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

In some embodiments, the transposase may be a Tn5 transposase. In some embodiments, the transposase may be a variant of a Tn5 transposase, or an engineered transposase. Transposases may be engineered using any method known in the art. The engineered transposase may be optimized to function at a temperature ranging from 30° C. to 45° C., 35° C. to 40° C. or any temperature in between, such as 30, 31, 32, 33, 34, 35, 36, 37, 38, 39, 40, 41, 42, 43, 44, to/or 45° C. In some embodiments, The engineered transposase may be optimized to release from the oligonucleotide at a faster rate compared to a wild type transposase.

In some embodiments, the transposase may be a Tn5 transposase, a Mu transposase, or a Tn7 transposase. Transposition efficiency in vitro may vary depending on the transposon system used. Generally, Tn5 and Mu transposases effect higher levels of transposition efficiency. In some embodiments, insertion may be random. In some embodiments, insertion may occur in GC rich regions of the target sequence.

In some embodiments, the transposon sequence may comprise two 19 base pair Mosaic End (ME) Tn5 transposase recognition sequences. Tn5 transposases will generally transpose any DNA sequence contained between such short 19 base pair ME Tn5 transposase recognition sequences.

In some embodiments, use of a transposase allows for separation of a double-stranded polynucleotide in the absence of heat or melting. Embodiments can be as described in International Application Publication WO 2020/00604, which is incorporated herein by reference.

### Nickase Dependent Amplification

In an embodiment of the invention may comprise nickase-based amplification. The nicking enzyme may be a CRISPR protein. Accordingly, the introduction of nicks into dsDNA can be programmable and sequence-specific. In an embodiment of the invention, two guides can be designed to target opposite strands of a dsDNA target. According to the invention, the nickase can be Cas12a, Cas12b, Cas9 or any ortholog or CRISPR protein that cleaves or is engineered to cleave a single strand of a DNA duplex. The nicked strands may then be extended by a polymerase. In an embodiment, the locations of the nicks are selected such that extension of the strands by a polymerase is towards the central portion of the target duplex DNA between the nick sites. In certain embodiments, primers are included in the reaction capable of hybridizing to the extended strands followed by further polymerase extension of the primers to regenerate two dsDNA pieces: a first dsDNA that includes the first strand Cas12a guide site or both the first and second strand Cas12a guide sites, and a second dsDNA that includes the second strand Cas12a guide site or both the first and second strand Cas12a guide sites. These pieces continue to be nicked and extended in a cyclic reaction that exponentially amplifies the region of the target between nicking sites.

The amplification can be isothermal and selected for temperature. In one embodiment, the amplification proceeds rapidly at 37° C. In other embodiments, the temperature of the isothermal amplification may be chosen by selecting a polymerase (e.g., Bsu, Bst, Phi29, klenow fragment etc.). operable at a different temperature.

Thus, whereas nicking isothermal amplification techniques use nicking enzymes with fixed sequence preference (e.g., in nicking enzyme amplification reaction or NEAR), which requires denaturing of the original dsDNA target to allow annealing and extension of primers that add the nicking substrate to the ends of the target, use of a CRISPR nickase wherein the nicking sites can be programed via guide RNAs means that no denaturing step is necessary, enabling the entire reaction to be truly isothermal. This also simplifies the reaction because these primers that add the nicking substrate are different than the primers that are used later in the reaction, meaning that NEAR requires two primer sets (i.e., 4 primers) while Cpf1 nicking amplification only requires one primer set (i.e., two primers). This makes nicking Cpf1 amplification much simpler and easier to operate without complicated instrumentation to perform the denaturation and then cooling to the isothermal temperature.

In an aspect, the isothermal amplification reagents may be utilized with a thermostable CRISPR-Cas protein. The combination of thermostable protein and isothermal amplification reagents may be utilized to further improve reaction times for detection and diagnostics. Embodiments can be as described in International Application Publication WO 2020/006067, entitled CRISPR Double Nickase Based Amplification Compositions, Systems and Methods, incorporated herein by reference.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM to 1 M, such as about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 975 mM, to/or 1 M, or the like. One of ordinary skill in the art will be able to determine an appropriate concentration of a buffer such as Tris for use with the present invention.

### Helicase-Dependent Amplification

In helicase-dependent amplification (HDA), a helicase enzyme is used to unwind a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

The target nucleic acid may be amplified by opening R-loops of the target nucleic acid using first and second CRISPR/Cas complexes. The first and second strand of the target nucleic acid may thus be unwound using a helicase, allowing primers and polymerase to bind and extend the DNA under isothermal conditions.

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W. H. Freeman and Company (2nd ed. (1992)), especially chapter 11). Any helicase that translocates along DNA or RNA in a 5′ to 3′ direction or in the opposite 3′ to 5′ direction may be used in present embodiments of the invention. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2^{nd} ed. (1992)), include E. coli helicase I, II, III, & IV, Rep, DnaB, PriA, PcrA, T4 Gp41helicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem. 276:232-243 (2001)), thermostable UvrD helicases from T. tengcongensis (disclosed in this invention, Example XII) and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms ((Grainge et al., Nucleic Acids Res. 31:4888-4898 (2003)).

A traditional definition of a helicase is an enzyme that catalyzes the reaction of separating/unzipping/unwinding the helical structure of nucleic acid duplexes (DNA, RNA or hybrids) into single-stranded components, using nucleoside triphosphate (NTP) hydrolysis as the energy source (such as ATP). However, it should be noted that not all helicases fit this definition anymore. A more general definition is that they are motor proteins that move along the single-stranded or double stranded nucleic acids (usually in a certain direction, 3′ to 5′ or 5 to 3, or both), i.e., translocases, that can or cannot unwind the duplexed nucleic acid encountered. In addition, some helicases simply bind and “melt” the duplexed nucleic acid structure without an apparent translocase activity.

Helicases exist in all living organisms and function in all aspects of nucleic acid metabolism. Helicases are classified based on the amino acid sequences, directionality, oligomerization state and nucleic-acid type and structure preferences. The most common classification method was developed based on the presence of certain amino acid sequences, called motifs. According to this classification helicases are divided into 6 super families: SF1, SF2, SF3, SF4, SF5 and SF6. SF1 and SF2 helicases do not form a ring structure around the nucleic acid, whereas SF3 to SF6 do. Superfamily classification is not dependent on the classical taxonomy.

DNA helicases are responsible for catalyzing the unwinding of double-stranded DNA (dsDNA) molecules to their respective single-stranded nucleic acid (ssDNA) forms. Although structural and biochemical studies have shown how various helicases can translocate on ssDNA directionally, consuming one ATP per nucleotide, the mechanism of nucleic acid unwinding and how the unwinding activity is regulated remains unclear and controversial (T. M. Lohman, E. J. Tomko, C. G. Wu, “Non-hexameric DNA helicases and translocases: mechanisms and regulation,” Nat Rev Mol Cell Biol 9:391-401 (2008)). Since helicases can potentially unwind all nucleic acids encountered, understanding how their unwinding activities are regulated can lead to harnessing helicase functions for biotechnology applications.

The term “HDA” refers to Helicase Dependent Amplification, which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification. Embodiments can be as described in International Application PCT/US2019/039167, entitled CRISPR Effector System Based Amplification Methods, Systems and Diagnostics.

The invention comprises use of any suitable helicase known in the art. These include, but are not necessarily limited to, UvrD helicase, CRISPR-Cas3 helicase, E. coli helicase I, E. coli helicase II, E. coli helicase III, E. coli helicase IV, Rep helicase, DnaB helicase, PriA helicase, PcrA helicase, T4 Gp41 helicase, T4 Dda helicase, SV40 Large T antigen, yeast RAD helicase, RecD helicase, RecQ helicase, thermostable T. tengcongensis UvrD helicase, thermostable T. thermophilus UvrD helicase, thermostable T. aquaticus DnaB helicase, Dda helicase, papilloma virus E1 helicase, archaeal MCM helicase, eukaryotic MCM helicase, and T7 Gp4 helicase.

In particularly preferred embodiments, the helicase comprises a super mutation. In particular embodiments, Although the E coli mutation has been described, the mutations were generated by sequence alignment (e.g. D409A/D410A for TteUvrd) and result in thermophilic enzymes working at lower temperatures like 37 C, which is advantageous for amplification methods and systems described herein. In some embodiments, the super mutation is an aspartate to alanine mutation, with position based on sequence alignment. In some embodiments, the super mutant helicase is selected from WP 003870487.1 Thermoanaerobacter ethanolicus 403/404, WP_049660019.1 Bacillus sp. FJAT-27231 407/408, WP_034654680.1 Bacillus megaterium 415/416, WP 095390358.1 Bacillus simplex 407/408, and WP 055343022.1 Paeniclostridium sordellii 402/403.

Methods of amplifying, detection and/or quantifying using the systems disclosed herein can comprise incubating the sample or set of samples under conditions sufficient for an enzymatic reaction to occur. In certain example embodiments, the incubation time of the present invention may be shortened. One skilled in the art can perform biochemical reactions in 5 minutes (e.g., 5 minute ligation). Incubating may occur at one or more temperatures over time frames between about 10 minutes and 3 hours, preferably less than 200 minutes, 150 minutes, 100 minutes, 75 minutes, 60 minutes, 45 minutes, 30 minutes, or 20 minutes, depending on sample, reagents and components of the system. In some embodiments, incubating is performed at one or more temperatures between about 20° C. and 100° C., in certain embodiments or about 37° C., in some embodiments, between about 45° C. to 75° C., 55 to 70° C. or 60° C. to 65° C. In some embodiments, incubating is performed at one or more temperatures (such as in one or more incubation steps) at a temperature that is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100° C. In some embodiments, incubation is performed in one or more steps where each step is performed at a temperature ranging from about 20, to about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. In some embodiments, incubation is performed in one or more steps where each step is performed at a temperature ranging from about 45, to 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75° C. In some embodiments, incubation is performed in one or more steps where each step is performed at a temperature ranging from about 55 to 56, 57, 58, 59, 60, 61, 62, 63, 64, 65, 66, 67, 68, 69, or 70° C. In some embodiments, incubation is performed in one or more steps where each step is performed at a temperature ranging from about 60 to 61, 62, 63, 64, or 65° C.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

### Other General Amplification Reagent Considerations

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected.

Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In certain example embodiments, the kit may further include a colorimetric or turbidimetric indicator with generation of a color signal or increase in turbidity of reaction indicating successful amplification of the one or more target molecule.

### Lysis Reagents

Other components of a biological or chemical reaction may include a cell lysis component in order to break open or lyse a cell for analysis of the materials therein. A cell lysis component(s) may include, but are not limited to, a detergent, a salt as described above, such as NaCl, KCl, ammonium sulfate [(NH4)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40), Polysorbate 10, Polysorbate 20, Proteinase K, Saponin. Concentrations of detergents may depend on the particular application, and may be specific to the reaction in some cases.

In certain example embodiments, the lysis reagent may be the Quick Extract lysis reagents described in e.g., Example 6, which does not require isolation of the nucleic acid prior to further processing of the sample.

### Additional Reagents

The kits can also include additional reagents, such as protein inhibitors, reaction indicators capable of providing a detectable signal of reaction status and/or result, suitable control reagents, combinations thereof, and the like. In some embodiments, the reaction indicator is a colorimetric or turbidimetric indicator. In some embodiments, the reaction indicator is a pH sensitive indicator. In some embodiments, the control reagents are capable of amplifying a control target in a sample. Suitable control targets include housekeeping targets, or other targets that are expected to be expressed or are otherwise capable of being detected in all samples tested. In some embodiments, the suitable control targets are also typically expressed or otherwise capable of detection at about the same level or amount or at about the same relative level or relative amount (such as relative to sample weight or volume on a total, liquid, total protein, total nucleic acid, total DNA, total RNA, or dry matter basis).

### Sample Collection Component

The kit can include and/or the method can employ, in some embodiments, a sample collection component. The sample collection component may comprise one or more components to be used by a patient or health care worker. The sample collection may include a nasopharyngeal swab, oropharyngeal swab, or other swab for collection of an oral or nasal sample. Sterile polyester or nylon swabs with aluminum or plastic shafts are preferable. Squirt bottles, bulbs, syringes or other means for rinsing of an oral or nasal cavity can be provided as a sample collection component. The bulb, bottle, or syringe may be provided pre-loaded with saline for the collection of a specimen. A sterile suction catheter suction apparatus can be provided for obtention of a nasopharyngeal/nasal aspirate. Jars and other sample containers can be used for collection of stool samples, and may further include additional probes, swabs and tubes for further sample processing. In some embodiments, the sample collection component is configured for collection of a nasal swab, an oral swab, a nasal wash, an oral wash, a fecal sample, a wound swab, or a combination thereof.

Sample collection components may comprise a vial, tube or other containment means for the collected sample. The sample collection component may comprise tubes, vials, containers or other receptacles for the collection of a sample wash, for example, nasal or oral washes. The containment means may comprise a lid configured to receive all or a portion of a swab, or may be configured such that the swab is provided as a portion of the lid to the containment means. The containment may comprise one or more reagents, including amplification reagents, solvents, detergents and other solutions, and can be designed for use in further sample manipulations, shipping, and subsequent reactions. In a preferred embodiment, one or more of the sample collection components is resistant to heat, and the collected sample can be further reacted and processed within the sample collection component, for example, for conducting the isothermal amplification reaction. Sample collection means may be further provided with ice packs and other shipping packaging.

In some embodiments, the sample collection component can be or include a sample dosing component. The sample dosing component can be configured to portion out or separate out a portion a collected sample to a desired amount (e.g., a dose appropriate for the downstream reaction(s)) and optionally facilitate placement of the portion in a reaction or other collection vessel. Non-limiting sample dosing components are spoons, spatulas, depression sticks, droppers, capillaries, syringes, and the like. In some embodiments, the sample dosing component can be part of or form the sample collection component. In some embodiments, the sample dosing component and the sample dosing component are separate components of the kit.

### Heating Component

The kits can include one or more heating components that can be utilized by the patient or health care worker. In embodiments, the heating component is a chemical heating regent, for example, molten sodium acetate, or air-activated packets comprising iron powder. The chemical heating reagent may be provided in a shape for receiving the sample collection component of the reaction vessel. The heating component may comprise a heating element, such as a cartridge or coil heating element, or the heating component may be provided by the end user. When the heating component is an end-user supplied heating element the kit may further comprise instructions for the use of readily available heating components for use by a patient or health care worker, including microwaves, ovens, driers, heating plates, pads blocks, and incubators, and may further comprise a thermometer for measuring heating temperatures.

In some embodiments, the one or more heating components, such as a chemical heating reagent, is/are configured for use in a reaction, such as an isothermal reaction conducted at a temperature of between 45° C. to 75° C., such as any temperature of about 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 degrees C., including any value or range of values therein. In some embodiments, the one or more heating components, such as a chemical heating reagent, is/are configured for use in a reaction, such as an isothermal reaction conducted at a temperature of between 55° C. to 70° C. the one or more heating components, such as a chemical heating reagent, is/are configured for use in a reaction, such as an isothermal reaction conducted at a temperature of between 60° C. to 65° C.

### Reaction Vessel

The reaction vessel may be one of the sample collection components described above, or may be a separate vessel suitable for the storing and shipping of a sample. The reaction vessel may comprise pre-mixed ingredients, including amplification reagents, detergents, sterile solutions, and other reagents that may be utilized in further sample processing. In certain embodiments, the reaction vessel is configured to receive a sample collection component above, such as a swab. As an example, the reaction vessel may comprise a lid configured to receive all or a portion of a swab, or may be configured such that the swab is provided as a portion of the lid of the reaction vessel. The reaction vessel may be pre-loaded with sterile solution for the storing of the sample. More than one reaction vessel may be included if more than one target is to be detected. Each reaction vessel can comprise reagents specific for the detection of each separate target. Reagents may be provided lyophilized, with reconstitution at the point of use, for example with a solution provided as a separate element of the kit. The reaction vessel is preferably designed to be heated and can be configured for use with particular heating elements and/or type of end-user.

The reaction vessel may be suitable for processing of individual samples or may comprise multi-well plates capable of processing multiple samples simultaneously.

In some embodiments, the reaction vessel includes a pre-mixed combination of amplification reagents and barcoded primers and configured to be sealed after receiving the sample, sample collection component, sample dosing component, or a combination thereof.

In some embodiments, the reaction vessel is configured for use in an isothermal amplification reaction. In some embodiments, the reaction vessel is configured for use at a point of care. In some embodiments, the reaction vessel is configured for use in an isothermal amplification reaction conducted at a point of care.

### Methods of Parallel Processing of Pooled Samples

In another aspect, the embodiments disclosed herein are directed to methods for parallel detection of one or more target sequence across multiple pooled samples. The methods can be adapted for use with the kits described above, but can also be applied to samples obtained by other means. The method utilizes a compressed sensing space of barcodes that is the number of unique barcode sequences needed to cover a particular use and can be set to a determined size. Accordingly, the method is advantageously scalable to a particular sample size, allowing for high-throughput processing of a large numbers of unique samples. The methods are designed to be used with samples that have undergone an initial heating/amplification step to label target sequences with a unique combination of barcodes. Individual barcodes may be used across multiple kits, but each sample can be labeled with a unique combination of barcodes. In this way, the set of individual barcodes needed can be much smaller than if a unique barcode had to be assigned to each sample to be processed. The method can process multiple individual samples in parallel by pooling individual samples that have been labeled with unique combinations of individual barcodes into pools.

The number of barcodes that are to be used to label an individual sample is determined at least in part by the total number of individual barcodes in a defined barcode set and the number of samples to be processed in parallel. The pool size, or number of samples that can be pooled together for processing by the methods disclosed herein, is optimized based on an expected or empirically determined fraction of positive samples, an estimated or empirically determined fraction of ineffective barcodes, a frequency of sample barcode dropout, a heterogeneity of sample representation in sequencing data, a false-positive cutoff rate, a false-negative cutoff rate, or a combination thereof. Example methods for determining an appropriate size of the barcode set to be used and methods for optimizing pool size are described in further detail below in e.g., Example 3.

In some embodiments, a defined barcode set for any given method or kit can be designed and/or configured according to a set of any number of kits such that unique samples analyzed by the set of kits can be processed in parallel according to the methods herein and still be uniquely identified. In some embodiments, the number of kits can range from 1 to 1,000 or more, such as 1, 2, 3, 4, 5, 6, 7, 8, 9, 10, 0, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 or more. In some embodiments, the barcodes within a set of kits can include a set of barcodes unique to each kit within the set.

The method can take a large set of individual samples as its initial input. These samples may have previously been processed individually to undergo a labeling step, whereby one or more target sequences are initially amplified to label with at least one barcodes as noted above. In certain example embodiments, the samples have been labeled with two or more barcodes to generate a unique combination of barcodes. This initial amplification step can be an isothermal amplification step. This initial labeling and/or amplification step can completed at a separate location from one or more other steps in the method, for example by an end user, sample collection point, and/or point of care health provider, that had been provided with a kit for that purpose. In certain example embodiments, the initial amplification/labeling is conducted using one of the kits disclosed herein.

After initial amplification/labeling, the samples can be pooled together for further processing and/or amplification steps and/or sequencing steps. As noted above, the number of pools is a function of samples to be processed and other optimization factors. Example guidance on determining the appropriate pool size is provided in the Working Examples below, such as in Examples 3, 7 and 8. In certain example embodiments, the number of pooled sample sets may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In certain example embodiments, the number of pooled sample sets may be 384.

Each pooled sample set is then processed in bulk. In some embodiments, a further amplification reaction is carried out on the pooled sample set to further amplify an amplicon comprising the one or more target sequences and the barcode combination. The further amplification reaction may be a PCR primer amplification. Appropriate primers for the PCR reaction can be designed using known techniques in the art and depending on the target polynucleotide to be amplified. In certain example embodiments, the primers used may bind to a portion of the target nucleic acid sequence not covered, or only partially covered, by the isothermal amplification primers. In certain example embodiment, the amplicon resulting from the further PCR amplification step, particularly where the isothermal amplification or initial amplification step was a LAMP-based isothermal amplification stem, may span one, or preferably all, junctions between a barcode sequence and the target nucleic acid sequence. In certain example embodiments, the further amplification reaction may incorporate at least one additional barcode into the amplicon. The at least one additional barcode can identify the pooled set in which a given sample was processed. In other words, the at least one additional barcode is unique to the pooled set in which a given sample was processed.

In certain example embodiments, the pooled samples are first diluted prior to the amplification reaction (e.g., PCR reaction) on the pooled samples. In some embodiments, the dilution is sufficient to reduce the formation of aberrant amplification products that would be produced from primers designed for the initial amplification step. In certain example embodiments, the pooled samples are diluted between 1:1,000 to 1:1,000,000 prior to further amplification. Exemplary dilutions include, without limitation 1 to 1,000, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, 110000, 111000, 112000, 113000, 114000, 115000, 116000, 117000, 118000, 119000, 120000, 121000, 122000, 123000, 124000, 125000, 126000, 127000, 128000, 129000, 130000, 131000, 132000, 133000, 134000, 135000, 136000, 137000, 138000, 139000, 140000, 141000, 142000, 143000, 144000, 145000, 146000, 147000, 148000, 149000, 150000, 151000, 152000, 153000, 154000, 155000, 156000, 157000, 158000, 159000, 160000, 161000, 162000, 163000, 164000, 165000, 166000, 167000, 168000, 169000, 170000, 171000, 172000, 173000, 174000, 175000, 176000, 177000, 178000, 179000, 180000, 181000, 182000, 183000, 184000, 185000, 186000, 187000, 188000, 189000, 190000, 191000, 192000, 193000, 194000, 195000, 196000, 197000, 198000, 199000, 200000, 201000, 202000, 203000, 204000, 205000, 206000, 207000, 208000, 209000, 210000, 211000, 212000, 213000, 214000, 215000, 216000, 217000, 218000, 219000, 220000, 221000, 222000, 223000, 224000, 225000, 226000, 227000, 228000, 229000, 230000, 231000, 232000, 233000, 234000, 235000, 236000, 237000, 238000, 239000, 240000, 241000, 242000, 243000, 244000, 245000, 246000, 247000, 248000, 249000, 250000, 251000, 252000, 253000, 254000, 255000, 256000, 257000, 258000, 259000, 260000, 261000, 262000, 263000, 264000, 265000, 266000, 267000, 268000, 269000, 270000, 271000, 272000, 273000, 274000, 275000, 276000, 277000, 278000, 279000, 280000, 281000, 282000, 283000, 284000, 285000, 286000, 287000, 288000, 289000, 290000, 291000, 292000, 293000, 294000, 295000, 296000, 297000, 298000, 299000, 300000, 301000, 302000, 303000, 304000, 305000, 306000, 307000, 308000, 309000, 310000, 311000, 312000, 313000, 314000, 315000, 316000, 317000, 318000, 319000, 320000, 321000, 322000, 323000, 324000, 325000, 326000, 327000, 328000, 329000, 330000, 331000, 332000, 333000, 334000, 335000, 336000, 337000, 338000, 339000, 340000, 341000, 342000, 343000, 344000, 345000, 346000, 347000, 348000, 349000, 350000, 351000, 352000, 353000, 354000, 355000, 356000, 357000, 358000, 359000, 360000, 361000, 362000, 363000, 364000, 365000, 366000, 367000, 368000, 369000, 370000, 371000, 372000, 373000, 374000, 375000, 376000, 377000, 378000, 379000, 380000, 381000, 382000, 383000, 384000, 385000, 386000, 387000, 388000, 389000, 390000, 391000, 392000, 393000, 394000, 395000, 396000, 397000, 398000, 399000, 400000, 401000, 402000, 403000, 404000, 405000, 406000, 407000, 408000, 409000, 410000, 411000, 412000, 413000, 414000, 415000, 416000, 417000, 418000, 419000, 420000, 421000, 422000, 423000, 424000, 425000, 426000, 427000, 428000, 429000, 430000, 431000, 432000, 433000, 434000, 435000, 436000, 437000, 438000, 439000, 440000, 441000, 442000, 443000, 444000, 445000, 446000, 447000, 448000, 449000, 450000, 451000, 452000, 453000, 454000, 455000, 456000, 457000, 458000, 459000, 460000, 461000, 462000, 463000, 464000, 465000, 466000, 467000, 468000, 469000, 470000, 471000, 472000, 473000, 474000, 475000, 476000, 477000, 478000, 479000, 480000, 481000, 482000, 483000, 484000, 485000, 486000, 487000, 488000, 489000, 490000, 491000, 492000, 493000, 494000, 495000, 496000, 497000, 498000, 499000, 500000, 501000, 502000, 503000, 504000, 505000, 506000, 507000, 508000, 509000, 510000, 511000, 512000, 513000, 514000, 515000, 516000, 517000, 518000, 519000, 520000, 521000, 522000, 523000, 524000, 525000, 526000, 527000, 528000, 529000, 530000, 531000, 532000, 533000, 534000, 535000, 536000, 537000, 538000, 539000, 540000, 541000, 542000, 543000, 544000, 545000, 546000, 547000, 548000, 549000, 550000, 551000, 552000, 553000, 554000, 555000, 556000, 557000, 558000, 559000, 560000, 561000, 562000, 563000, 564000, 565000, 566000, 567000, 568000, 569000, 570000, 571000, 572000, 573000, 574000, 575000, 576000, 577000, 578000, 579000, 580000, 581000, 582000, 583000, 584000, 585000, 586000, 587000, 588000, 589000, 590000, 591000, 592000, 593000, 594000, 595000, 596000, 597000, 598000, 599000, 600000, 601000, 602000, 603000, 604000, 605000, 606000, 607000, 608000, 609000, 610000, 611000, 612000, 613000, 614000, 615000, 616000, 617000, 618000, 619000, 620000, 621000, 622000, 623000, 624000, 625000, 626000, 627000, 628000, 629000, 630000, 631000, 632000, 633000, 634000, 635000, 636000, 637000, 638000, 639000, 640000, 641000, 642000, 643000, 644000, 645000, 646000, 647000, 648000, 649000, 650000, 651000, 652000, 653000, 654000, 655000, 656000, 657000, 658000, 659000, 660000, 661000, 662000, 663000, 664000, 665000, 666000, 667000, 668000, 669000, 670000, 671000, 672000, 673000, 674000, 675000, 676000, 677000, 678000, 679000, 680000, 681000, 682000, 683000, 684000, 685000, 686000, 687000, 688000, 689000, 690000, 691000, 692000, 693000, 694000, 695000, 696000, 697000, 698000, 699000, 700000, 701000, 702000, 703000, 704000, 705000, 706000, 707000, 708000, 709000, 710000, 711000, 712000, 713000, 714000, 715000, 716000, 717000, 718000, 719000, 720000, 721000, 722000, 723000, 724000, 725000, 726000, 727000, 728000, 729000, 730000, 731000, 732000, 733000, 734000, 735000, 736000, 737000, 738000, 739000, 740000, 741000, 742000, 743000, 744000, 745000, 746000, 747000, 748000, 749000, 750000, 751000, 752000, 753000, 754000, 755000, 756000, 757000, 758000, 759000, 760000, 761000, 762000, 763000, 764000, 765000, 766000, 767000, 768000, 769000, 770000, 771000, 772000, 773000, 774000, 775000, 776000, 777000, 778000, 779000, 780000, 781000, 782000, 783000, 784000, 785000, 786000, 787000, 788000, 789000, 790000, 791000, 792000, 793000, 794000, 795000, 796000, 797000, 798000, 799000, 800000, 801000, 802000, 803000, 804000, 805000, 806000, 807000, 808000, 809000, 810000, 811000, 812000, 813000, 814000, 815000, 816000, 817000, 818000, 819000, 820000, 821000, 822000, 823000, 824000, 825000, 826000, 827000, 828000, 829000, 830000, 831000, 832000, 833000, 834000, 835000, 836000, 837000, 838000, 839000, 840000, 841000, 842000, 843000, 844000, 845000, 846000, 847000, 848000, 849000, 850000, 851000, 852000, 853000, 854000, 855000, 856000, 857000, 858000, 859000, 860000, 861000, 862000, 863000, 864000, 865000, 866000, 867000, 868000, 869000, 870000, 871000, 872000, 873000, 874000, 875000, 876000, 877000, 878000, 879000, 880000, 881000, 882000, 883000, 884000, 885000, 886000, 887000, 888000, 889000, 890000, 891000, 892000, 893000, 894000, 895000, 896000, 897000, 898000, 899000, 900000, 901000, 902000, 903000, 904000, 905000, 906000, 907000, 908000, 909000, 910000, 911000, 912000, 913000, 914000, 915000, 916000, 917000, 918000, 919000, 920000, 921000, 922000, 923000, 924000, 925000, 926000, 927000, 928000, 929000, 930000, 931000, 932000, 933000, 934000, 935000, 936000, 937000, 938000, 939000, 940000, 941000, 942000, 943000, 944000, 945000, 946000, 947000, 948000, 949000, 950000, 951000, 952000, 953000, 954000, 955000, 956000, 957000, 958000, 959000, 960000, 961000, 962000, 963000, 964000, 965000, 966000, 967000, 968000, 969000, 970000, 971000, 972000, 973000, 974000, 975000, 976000, 977000, 978000, 979000, 980000, 981000, 982000, 983000, 984000, 985000, 986000, 987000, 988000, 989000, 990000, 991000, 992000, 993000, 994000, 995000, 996000, 997000, 998000, 999000 or 1,000,000.

Each pooled set of barcoded amplicons is then sequenced using a suitable sequencing technique. In certain example embodiments, the pooled sets of barcoded amplicons may be sequences using a next generation sequencing technologies. Exemplary next generation sequencing technologies include, for example, Illumina sequencing, Ion Torrent sequencing, 454 sequencing, SOLiD sequencing, and nanopore sequencing amongst others.

The sequencing readout will include the target sequence and the barcode sequences. The target sequence identifies the presence of the target sequence and the barcode combination allows for identification of the individual sample (subject) testing positive for the particular target sequence.

In some embodiments, one or more reagents are included in the reaction, such that a visible or otherwise detectable signal is produced (e.g., a change in color or turbidity) so as to indicate a positive amplification (either of a target or control). Such signal can be indicative of the reaction completion (or incompletion), positive (or negative) results, and the like. Such a signal can be qualitative or quantitative. Such a signal can be observed to the naked eye and/or can be detected using a suitable detection device, including but not limited to a smart phone, tablet, or other handheld device, computer, or other suitable sensor/detector.

In some exemplary embodiments, the method of parallel detection of one or more target sequence across multiple samples includes (a) separating a set of samples into one or more pooled sample sets, wherein each sample comprises of the set of samples includes an initial amplicon that includes one or more target sequences and at least one barcode; (b) conducting an amplification reaction on the one or more pooled sets of (a) to further amplify the amplicons, and optionally, further incorporating one or more additional barcodes to the amplicon; (c) sequencing the amplicons after (b); and identifying individual samples from the pooled sample set that are positive for the one or more target sequences based on sequencing of the amplicons in (c), wherein identification is based, at least in part, on detection of the unique combination of barcodes present in the sequenced amplicons. In some embodiments, at least one barcode in the initial is unique to the sample, an initial amplification reaction primer, an initial amplification reaction primer set, or a combination thereof. In some embodiments, at least one of the one or more additional barcodes is unique to the pooled set.

In some embodiments, the amplicon resulting from amplification of the pooled sets spans a fraction of the target nucleic acid sequence not covered or only partially covered by the primers used to generate an initial amplicon. In some embodiments, the amplicon resulting from the amplification of pooled sets spans one, or preferably both, of the junctions between a barcode sequence and the target nucleic acid sequence.

In some embodiments, sequencing the amplicons comprises deep sequencing of the amplicons.

In some embodiments, the set of samples is/are diluted to between 1:1,000 to 1:1,000,000 prior to the amplification reaction of (b).

In some embodiments, the method further comprises generating one or more initial amplicons before step (a) by amplifying one or more amplicons in each individual samples wherein one or more amplicons include one or more target sequences. In some embodiments, generating one or more initial amplicons is or includes an isothermal reaction. In some embodiments, generating one or more initial amplicons is or includes LAMP or RT-LAMP.

In some embodiments, the amplicons in each individual sample comprising the set of samples of step (a) are generated by conducting an isothermal amplification reaction on each individual sample using one or more primer sets and wherein a primer in each primer set comprises a barcode and each set of primers comprises a combination of barcodes unique to each sample. In some embodiments, one or more of the barcode sequences are inserted between two target-specific sequences of either a forward inner primer (FIP), a backward inner primer (BIP), or both.

In some embodiments, the number of barcodes used per sample is determined, at least in part, on the total number of barcode sequences in a defined set of barcode sequences and a number of samples to be processed in parallel. The Working Examples provided herein (including, but not limited to, Examples 3, 7, and 8) a demonstration and modeling of determining the minimum number of barcodes needed based on the number of samples to be processed and analyzed in parallel. In some embodiments, the number of barcodes used per sample is between 1 and 20, and more particularly, 2 and 20, 3 and 20, 4 and 20, 5 and 20, 6 and 20, 7 and 20, 8 and 20, 9 and 20, 10 and 20, 11 and 20, 12 and 20, 13 and 20, 14 and 20, 15 and 20, 16 and 20, 17 and 20, 18 and 20 or 19 and 20.

In some embodiments, the barcodes, such as the barcodes for an initial amplification, are selected so as to avoid barcodes comprising a sequence portion that is a reverse complement to the 3′ end of a primer, in particular the 3′ end of a forward inner primer (FIP).

In some embodiments, the number of samples to be processed in a pooled set is optimized based on an expected or empirically determined fraction of positive samples, an estimated or empirically determined fraction of ineffective barcodes, a frequency of sample barcode dropout, a heterogeneity of sample representation in sequencing data, a false-positive cutoff rate, a false-negative cutoff rate, or a combination thereof.

In some embodiments, the samples are heat-inactivated wither prior to or after being pooled into a pooled sample set.

### Target Molecules and Uses

In certain example embodiments, the systems, devices, and methods disclosed herein may be used for biomarker detection. For example, the systems, devices and method disclosed herein may be used for SNP detection and/or genotyping. The systems, devices and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular, immune disorders, and cancer among other diseases may be detected. In certain example embodiments, the embodiments disclosed herein may be used for cell free DNA detection of diseases that involve lysis, such as liver fibrosis and restrictive/obstructive lung disease. In certain example embodiments, the embodiments could be utilized for faster and more portable detection for pre-natal testing of cell-free DNA.

### SNP & Genotyping Applications

The embodiments disclosed herein may be used for screening panels of different SNPs associated with, among others, cardiovascular health, lipid/metabolic signatures, ethnicity identification, paternity matching, human ID (e.g., matching suspect to a criminal database of SNP signatures). The embodiments disclosed herein may also be used for cell free DNA detection of mutations related to and released from cancer tumors. The embodiments disclosed herein may also be used for detection of meat quality, for example, by providing rapid detection of different animal sources in a given meat product. Embodiments disclosed herein may also be used for the detection of GMOs or gene editing related to DNA. As described herein elsewhere, closely related genotypes/alleles or biomarkers (e.g., having only a single nucleotide difference in a given target sequence) may be distinguished.

### Cancer and Cancer Drug Resistance Detection

In certain embodiments, the present invention may be used to detect genes and mutations associated with cancer. In certain embodiments, mutations associated with resistance are detected. The amplification of resistant tumor cells or appearance of resistant mutations in clonal populations of tumor cells may arise during treatment (see, e.g., Burger J A, et al., Clonal evolution in patients with chronic lymphocytic leukaemia developing resistance to BTK inhibition. Nat Commun. 2016 May 20; 7:11589; Landau D A, et al., Mutations driving CLL and their evolution in progression and relapse. Nature. 2015 Oct. 22;526(7574):525-30; Landau D A, et al., Clonal evolution in hematological malignancies and therapeutic implications. Leukemia. 2014 January;28(1):34-43; and Landau D A, et al., Evolution and impact of subclonal mutations in chronic lymphocytic leukemia. Cell. 2013 Feb. 14;152(4):714-26). Accordingly, detecting such mutations requires highly sensitive assays and monitoring requires repeated biopsy. Repeated biopsies are inconvenient, invasive and costly. Resistant mutations can be difficult to detect in a blood sample or other noninvasively collected biological sample (e.g., blood, saliva, urine) using the prior methods known in the art. Resistant mutations may refer to mutations associated with resistance to a chemotherapy, targeted therapy, or immunotherapy.

In certain embodiments, mutations occur in individual cancers that may be used to detect cancer progression. In one embodiment, mutations related to T cell cytolytic activity against tumors have been characterized and may be detected by the present invention (see e.g., Rooney et al., Molecular and genetic properties of tumors associated with local immune cytolytic activity, Cell. 2015 Jan. 15; 160(1-2): 48-61). Personalized therapies may be developed for a patient based on detection of these mutations (see e.g., International Patent Publication WO 2016100975A1). In certain embodiments, cancer specific mutations associated with cytolytic activity may be a mutation in a gene selected from the group consisting of CASP8, B2M, PIK3CA, SMC1A, ARIDSB, TET2, ALPK2, COL5A1, TP53, DNER, NCOR1, MORC4, CIC, IRF6, MYOCD, ANKLE1, CNKSR1, NF1, SOS1, ARID2, CUL4B, DDX3X, FUBP1, TCP11L2, HLA-A, B or C, CSNK2A1, MET, ASXL1, PD-L1, PD-L2, IDO1, IDO2, ALOX12B and ALOX15B, or copy number gain, excluding whole-chromosome events, impacting any of the following chromosomal bands: 6q16.1-q21, 6q22.31-q24.1, 6q25.1-q26, 7p11.2-q11.1, 8p23.1, 8p11.23-p11.21 (containing IDOL IDO2), 9p24.2-p23 (containing PDL1, PDL2), 10p15.3, 10p15.1-p13, 11p14.1, 12p13.32-p13.2, 17p13.1 (containing ALOX12B, ALOX15B), and 22q11.1-q11.21.

In certain embodiments, the present invention is used to detect a cancer mutation (e.g., resistance mutation) during the course of a treatment and after treatment is completed. The sensitivity of the present invention may allow for noninvasive detection of clonal mutations arising during treatment and can be used to detect a recurrence in the disease.

In certain example embodiments, detection of microRNAs (miRNA) and/or miRNA signatures of differentially expressed miRNA, may be used to detect or monitor progression of a cancer and/or detect drug resistance to a cancer therapy. As an example, Nadal et al. (Nature Scientific Reports, (2015) doi:10.1038/srep12464) describe mRNA signatures that may be used to detect non-small cell lung cancer (NSCLC).

In certain example embodiments, the presence of resistance mutations in clonal subpopulations of cells may be used in determining a treatment regimen. In other embodiments, personalized therapies for treating a patient may be administered based on common tumor mutations. In certain embodiments, common mutations arise in response to treatment and lead to drug resistance. In certain embodiments, the present invention may be used in monitoring patients for cells acquiring a mutation or amplification of cells harboring such drug resistant mutations.

Treatment with various chemotherapeutic agents, particularly with targeted therapies such as tyrosine kinase inhibitors, frequently leads to new mutations in the target molecules that resist the activity of the therapeutic. Multiple strategies to overcome this resistance are being evaluated, including development of second generation therapies that are not affected by these mutations and treatment with multiple agents including those that act downstream of the resistance mutation. In an exemplary embodiment, a common mutation to ibrutinib, a molecule targeting Bruton's Tyrosine Kinase (BTK) and used for CLL and certain lymphomas, is a Cysteine to Serine change at position 481 (BTK/C481S). Erlotinib, which targets the tyrosine kinase domain of the Epidermal Growth Factor Receptor (EGFR), is commonly used in the treatment of lung cancer and resistant tumors invariably develop following therapy. A common mutation found in resistant clones is a threonine to methionine mutation at position 790.

Non-silent mutations shared between populations of cancer patients and common resistant mutations that may be detected with the present invention are known in the art (see e.g., WO/2016/187508). In certain embodiments, drug resistance mutations may be induced by treatment with ibrutinib, erlotinib, imatinib, gefitinib, crizotinib, trastuzumab, vemurafenib, RAF/MEK, checkpoint blockade therapy, or antiestrogen therapy. In certain embodiments, the cancer specific mutations are present in one or more genes encoding a protein selected from the group consisting of Programmed Death-Ligand 1 (PD-L1), androgen receptor (AR), Bruton's Tyrosine Kinase (BTK), Epidermal Growth Factor Receptor (EGFR), BCR-Abl, c-kit, PIK3CA, HER2, EML4-ALK, KRAS, ALK, ROS1, AKT1, BRAF, MEK1, MEK2, NRAS, RAC1, and ESR1.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Recently, gene expression in tumors and their microenvironments have been characterized at the single cell level (see e.g., Tirosh, et al. Dissecting the multicellular ecosystem of metastatic melanoma by single cell RNA-seq. Science 352, 189-196, doi:10.1126/science.aad0501 (2016)); Tirosh et al., Single-cell RNA-seq supports a developmental hierarchy in human oligodendroglioma. Nature. 2016 Nov. 10;539(7628):309-313. doi: 10.1038/nature20123. Epub 2016 Nov. 2; and International patent publication serial number WO 2017004153 A1). In certain embodiments, gene signatures may be detected using the present invention. In one embodiment complement genes are monitored or detected in a tumor microenvironment. In one embodiment MITF and AXL programs are monitored or detected. In one embodiment, a tumor specific stem cell or progenitor cell signature is detected. Such signatures indicate the state of an immune response and state of a tumor. In certain embodiments, the state of a tumor in terms of proliferation, resistance to treatment and abundance of immune cells may be detected.

Thus, in certain embodiments, the invention provides low-cost, rapid, multiplexed cancer detection panels for circulating DNA, such as tumor DNA, particularly for monitoring disease recurrence or the development of common resistance mutations.

In certain embodiments, the systems, devices, and methods disclosed herein may be used for detecting the presence or expression level of long non-coding RNAs (lncRNAs). Expression of certain lncRNAs are associated with disease state and/or drug resistance. In particular, certain lncRNAs (e.g., TCONS_00011252, NR_034078, TCONS 00010506, TCONS 00026344, TCONS 00015940, TCONS 00028298, TCONS 00026380, TCONS 0009861, TCONS 00026521, TCONS 00016127, NR 125939, NR 033834, TCONS 00021026, TCONS 00006579, NR 109890, and NR 026873) are associated with resistance to cancer treatment, such as resistance to one or more BRAF inhibitors (e.g., Vemurafenib, Dabrafenib, Sorafenib, GDC-0879, PLX-4720, and LGX818) for treating melanoma (e.g., nodular melanoma, lentigo maligna, lentigo maligna melanoma, acral lentiginous melanoma, superficial spreading melanoma, mucosal melanoma, polypoid melanoma, desmoplastic melanoma, amelanotic melanoma, and soft-tissue melanoma). The detection of lncRNAs using the various embodiments described herein can facilitate disease diagnosis and/or selection of treatment options

### LOH Detection

Cancer cells undergo a loss of genetic material (DNA) when compared to normal cells. This deletion of genetic material which almost all, if not all, cancers undergo is referred to as “loss of heterozygosity” (LOH). Loss of heterozygosity (LOH) is a gross chromosomal event that results in loss of the entire gene and the surrounding chromosomal region. The loss of heterozygosity is a common occurrence in cancer, where it can indicate the absence of a functional tumor suppressor gene in the lost region. However, a loss may be silent because there still is one functional gene left on the other chromosome of the chromosome pair. The remaining copy of the tumor suppressor gene can be inactivated by a point mutation, leading to loss of a tumor suppressor gene. The loss of genetic material from cancer cells can result in the selective loss of one of two or more alleles of a gene vital for cell viability or cell growth at a particular locus on the chromosome.

An “LOH marker” is DNA from a microsatellite locus, a deletion, alteration, or amplification in which, when compared to normal cells, is associated with cancer or other diseases. An LOH marker often is associated with loss of a tumor suppressor gene or another, usually tumor related, gene.

The term “microsatellites” refers to short repetitive sequences of DNA that are widely distributed in the human genome. A microsatellite is a tract of tandemly repeated (i.e. adjacent) DNA motifs that range in length from two to five nucleotides, and are typically repeated 5-50 times. Somatic alterations in the repeat length of such microsatellites have been shown to represent a characteristic feature of tumors. Primers may be designed to detect such microsatellites. Furthermore, the present invention may be used to detect alterations in repeat length, as well as amplifications and deletions based upon quantitation of the detectable signal. Certain microsatellites are located in regulatory flanking or intronic regions of genes, or directly in codons of genes. Microsatellite mutations in such cases can lead to phenotypic changes and diseases, notably in triplet expansion diseases such as fragile X syndrome and Huntington's disease.

Frequent loss of heterozygosity (LOH) on specific chromosomal regions has been reported in many kinds of malignancies. Allelic losses on specific chromosomal regions are the most common genetic alterations observed in a variety of malignancies, thus microsatellite analysis has been applied to detect DNA of cancer cells in specimens from body fluids, such as sputum for lung cancer and urine for bladder cancer. (Rouleau, et al. Nature 363, 515-521 (1993); and Latif, et al. Science 260, 1317-1320 (1993)). Moreover, it has been established that markedly increased concentrations of soluble DNA are present in plasma of individuals with cancer and some other diseases, indicating that cell free serum or plasma can be used for detecting cancer DNA with microsatellite abnormalities. (Kamp, et al. Science 264, 436-440 (1994); and Steck, et al. Nat Genet. 15(4), 356-362 (1997)). Two groups have reported microsatellite alterations in plasma or serum of a limited number of patients with small cell lung cancer or head and neck cancer. (Hahn, et al. Science 271, 350-353 (1996); and Miozzo, et al. Cancer Res. 56, 2285-2288 (1996)). Detection of loss of heterozygosity in tumors and serum of melanoma patients has also been previously shown (see, e.g., United States patent number U.S. Pat. No. 6,465,177B1).

Thus, it is advantageous to detect LOH markers in a subject suffering from or at risk of cancer. The present invention may be used to detect LOH in tumor cells. In one embodiment, circulating tumor cells may be used as a biological sample. In preferred embodiments, cell free DNA obtained from serum or plasma is used to noninvasively detect and/or monitor LOH. In other embodiments, the biological sample may be any sample described herein (e.g., a urine sample for bladder cancer). Not being bound by a theory, the present invention may be used to detect LOH markers with improved sensitivity as compared to any prior method, thus providing early detection of mutational events. In one embodiment, LOH is detected in biological fluids, wherein the presence of LOH is associated with the occurrence of cancer. The method and systems described herein represents a significant advance over prior techniques, such as PCR or tissue biopsy by providing a non-invasive, rapid, and accurate method for detecting LOH of specific alleles associated with cancer. Thus, the present invention provides methods and systems which can be used to screen high-risk populations and to monitor high risk patients undergoing chemoprevention, chemotherapy, immunotherapy or other treatments.

### Circulating Tumor Cells

In one embodiment, circulating cells (e.g., circulating tumor cells (CTC)) can be assayed with the present invention. Isolation of circulating tumor cells (CTC) for use in any of the methods described herein may be performed. Exemplary technologies that achieve specific and sensitive detection and capture of circulating cells that may be used in the present invention have been described (Mostert B, et al., Circulating tumor cells (CTCs): detection methods and their clinical relevance in breast cancer. Cancer Treat Rev. 2009; 35:463-474; and Talasaz A H, et al., Isolating highly enriched populations of circulating epithelial cells and other rare cells from blood using a magnetic sweeper device. Proc Natl Acad Sci USA. 2009; 106:3970-3975). As few as one CTC may be found in the background of 105-106 peripheral blood mononuclear cells (Ross A A, et al., Detection and viability of tumor cells in peripheral blood stem cell collections from breast cancer patients using immunocytochemical and clonogenic assay techniques. Blood. 1993, 82:2605-2610). The CellSearch® platform uses immunomagnetic beads coated with antibodies to Epithelial Cell Adhesion Molecule (EpCAM) to enrich for EPCAM-expressing epithelial cells, followed by immunostaining to confirm the presence of cytokeratin staining and absence of the leukocyte marker CD45 to confirm that captured cells are epithelial tumor cells (Momburg F, et al., Immunohistochemical study of the expression of a Mr 34,000 human epithelium-specific surface glycoprotein in normal and malignant tissues. Cancer Res. 1987; 47:2883-2891; and Allard W J, et al., Tumor cells circulate in the peripheral blood of all major carcinomas but not in healthy subjects or patients with nonmalignant diseases. Clin Cancer Res. 2004; 10:6897-6904). The number of cells captured have been prospectively demonstrated to have prognostic significance for breast, colorectal and prostate cancer patients with advanced disease (Cohen S J, et al., J Clin Oncol. 2008; 26:3213-3221; Cristofanilli M, et al. N Engl J Med. 2004; 351:781-791; Cristofanilli M, et al., J Clin Oncol. 2005;23: 1420-1430; and de Bono J S, et al. Clin Cancer Res. 2008; 14:6302-6309).

The present invention also provides for isolating CTCs with CTC-Chip Technology. CTC-Chip is a microfluidic based CTC capture device where blood flows through a chamber containing thousands of microposts coated with anti-EpCAM antibodies to which the CTCs bind (Nagrath S, et al. Isolation of rare circulating tumor cells in cancer patients by microchip technology. Nature. 2007; 450: 1235-1239). CTC-Chip provides a significant increase in CTC counts and purity in comparison to the CellSearch® system (Maheswaran S, et al. Detection of mutations in EGFR in circulating lung-cancer cells, N Engl J Med. 2008; 359:366-377), both platforms may be used for downstream molecular analysis.

### Cell-Free Chromatin

In certain embodiments, cell free chromatin fragments are isolated and analyzed according to the present invention. Nucleosomes can be detected in the serum of healthy individuals (Stroun et al., Annals of the New York Academy of Sciences 906: 161-168 (2000)) as well as individuals afflicted with a disease state. Moreover, the serum concentration of nucleosomes is considerably higher in patients suffering from benign and malignant diseases, such as cancer and autoimmune disease (Holdenrieder et al (2001) Int J Cancer 95, 1 14-120, Trejo-Becerril et al (2003) Int J Cancer 104, 663-668; Kuroi et al 1999 Breast Cancer 6, 361-364; Kuroi et al (2001) Int j Oncology 19, 143-148; Amoura et al (1997) Arth Rheum 40, 2217-2225; Williams et al (2001) J Rheumatol 28, 81-94). Not being bound by a theory, the high concentration of nucleosomes in tumor bearing patients derives from apoptosis, which occurs spontaneously in proliferating tumors. Nucleosomes circulating in the blood contain uniquely modified histones. For example, U.S. Patent Publication No. 2005/0069931 (Mar. 31, 2005) relates to the use of antibodies directed against specific histone N-terminus modifications as diagnostic indicators of disease, employing such histone-specific antibodies to isolate nucleosomes from a blood or serum sample of a patient to facilitate purification and analysis of the accompanying DNA for diagnostic/screening purposes. Accordingly, the present invention may use chromatin bound DNA to detect and monitor, for example, tumor mutations. The identification of the DNA associated with modified histones can serve as diagnostic markers of disease and congenital defects.

Thus, in another embodiment, isolated chromatin fragments are derived from circulating chromatin, preferably circulating mono and oligonucleosomes. Isolated chromatin fragments may be derived from a biological sample. The biological sample may be from a subject or a patient in need thereof. The biological sample may be sera, plasma, lymph, blood, blood fractions, urine, synovial fluid, spinal fluid, saliva, circulating tumor cells or mucous.

Cell-Free DNA (cfDNA)

In certain embodiments, the present invention may be used to detect cell free DNA (cfDNA). Cell free DNA in plasma or serum may be used as a non-invasive diagnostic tool. For example, cell free fetal DNA has been studied and optimized for testing on-compatible RhD factors, sex determination for X-linked genetic disorders, testing for single gene disorders, identification of preeclampsia. For example, sequencing the fetal cell fraction of cfDNA in maternal plasma is a reliable approach for detecting copy number changes associated with fetal chromosome aneuploidy. For another example, cfDNA isolated from cancer patients has been used to detect mutations in key genes relevant for treatment decisions.

In certain example embodiments, the present disclosure provides detecting cfDNA directly from a patient sample. In certain other example embodiments, the present disclosure provides enriching cfDNA using the enrichment embodiments disclosed above and prior to detecting the target cfDNA.

### Pre-Natal Screening

In certain embodiments, the method and systems of the present invention may be used in prenatal screening. In certain embodiments, cell-free DNA is used in a method of prenatal screening. In certain embodiments, DNA associated with single nucleosomes or oligonucleosomes may be detected with the present invention. In preferred embodiments, detection of DNA associated with single nucleosomes or oligonucleosomes is used for prenatal screening. In certain embodiments, cell-free chromatin fragments are used in a method of prenatal screening.

Prenatal diagnosis or prenatal screening refers to testing for diseases or conditions in a fetus or embryo before it is born. The aim is to detect birth defects such as neural tube defects, Down syndrome, chromosome abnormalities, genetic disorders and other conditions, such as spina bifida, cleft palate, Tay Sachs disease, sickle cell anemia, thalassemia, cystic fibrosis, Muscular dystrophy, and fragile X syndrome. Screening can also be used for prenatal sex discernment. Common testing procedures include amniocentesis, ultrasonography including nuchal translucency ultrasound, serum marker testing, or genetic screening. In some cases, the tests are administered to determine if the fetus will be aborted, though physicians and patients also find it useful to diagnose high-risk pregnancies early so that delivery can be scheduled in a tertiary care hospital where the baby can receive appropriate care.

It has been realized that there are fetal cells which are present in the mother's blood, and that these cells present a potential source of fetal chromosomes for prenatal DNA-based diagnostics. Additionally, fetal DNA ranges from about 2-10% of the total DNA in maternal blood. Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses. Cell-free fetal DNA has been shown to exist in plasma and serum of pregnant women as early as the sixth week of gestation, with concentrations rising during pregnancy and peaking prior to parturition. Because these cells appear very early in the pregnancy, they could form the basis of an accurate, noninvasive, first trimester test. Not being bound by a theory, the present invention provides unprecedented sensitivity in detecting low amounts of fetal DNA. Not being bound by a theory, abundant amounts of maternal DNA is generally concomitantly recovered along with the fetal DNA of interest, thus decreasing sensitivity in fetal DNA quantification and mutation detection. The present invention overcomes such problems by the unexpectedly high sensitivity of the assay.

The H3 class of histones consists of four different protein types: the main types, H3.1 and H3.2; the replacement type, H3.3; and the testis specific variant, H3t. Although H3.1 and H3.2 are closely related, only differing at Ser96, H3.1 differs from H3.3 in at least 5 amino acid positions. Further, H3.1 is highly enriched in fetal liver, in comparison to its presence in adult tissues including liver, kidney and heart. In adult human tissue, the H3.3 variant is more abundant than the H3.1 variant, whereas the converse is true for fetal liver. The present invention may use these differences to detect fetal nucleosomes and fetal nucleic acid in a maternal biological sample that comprises both fetal and maternal cells and/or fetal nucleic acid.

In one embodiment, fetal nucleosomes may be obtained from blood. In other embodiments, fetal nucleosomes are obtained from a cervical mucus sample. In certain embodiments, a cervical mucus sample is obtained by swabbing or lavage from a pregnant woman early in the second trimester or late in the first trimester of pregnancy. The sample may be placed in an incubator to release DNA trapped in mucus. The incubator may be set at 37° C. The sample may be rocked for approximately 15 to 30 minutes. Mucus may be further dissolved with a mucinase for the purpose of releasing DNA. The sample may also be subjected to conditions, such as chemical treatment and the like, as well known in the art, to induce apoptosis to release fetal nucleosomes. Thus, a cervical mucus sample may be treated with an agent that induces apoptosis, whereby fetal nucleosomes are released. Regarding enrichment of circulating fetal DNA, reference is made to U.S. patent publication Nos. 20070243549 and 20100240054. The present invention is especially advantageous when applying the methods and systems to prenatal screening where only a small fraction of nucleosomes or DNA may be fetal in origin.

Prenatal screening according to the present invention may be for a disease including, but not limited to Trisomy 13, Trisomy 16, Trisomy 18, Klinefelter syndrome (47, XXY), (47, XYY) and (47, XXX), Turner syndrome, Down syndrome (Trisomy 21), Cystic Fibrosis, Huntington's Disease, Beta Thalassaemia, Myotonic Dystrophy, Sickle Cell Anemia, Porphyria, Fragile-X-Syndrome, Robertsonian translocation, Angelman syndrome, DiGeorge syndrome and Wolf-Hirschhorn Syndrome.

Several further aspects of the invention relate to diagnosing, prognosing and/or treating defects associated with a wide range of genetic diseases which are further described on the website of the National Institutes of Health under the topic subsection Genetic Disorders (website at health.nih.gov/topic/Genetic Disorders).

### Infectious Disease and Microbial Detection

In certain example embodiments, the systems, devices, and methods, disclosed herein are directed to detecting the presence of one or more microbial agents in a sample, such as a biological sample obtained from a subject. In certain example embodiments, the microbe may be a bacterium, a fungus, a yeast, a protozoa, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g., bacterial resistance), monitoring of disease progression and/or outbreak, and antibiotic screening. Because of the rapid and sensitive diagnostic capabilities of the embodiments disclosed here, detection of microbe species type, down to a single nucleotide difference, the embodiments disclosed herein may be used to guide therapeutic regimens, such as selection of the appropriate antibiotic or antiviral. The embodiments disclosed herein may also be used to screen environmental samples (air, water, surfaces, food etc.) for the presence of microbial contamination.

### Viral Detection

In certain example embodiments, the systems, devices, and methods, disclosed herein are directed to detecting viruses in a sample. The embodiments disclosed herein may be used to detect viral infection (e.g., of a subject or plant), or determination of a viral strain, including viral strains that differ by a single nucleotide polymorphism. The virus may be a DNA virus, a RNA virus, or a retrovirus. Non-limiting examples of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khuj and virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e., after infection has occurred or prophylactically, i.e., before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zoster virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among oIn some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

In certain embodiments, the virus is a drug resistant virus. By means of example, and without limitation, the virus may be a ribavirin resistant virus. Ribavirin is a very effective antiviral that hits a number of RNA viruses. Below are a few important viruses that have evolved ribavirin resistance. Foot and Mouth Disease Virus: doi:10.1128/JVI.03594-13. Polio virus: www.pnas.org/content/100/12/7289.full.pdf. Hepatitis C Virus: jvi.asm.org/content/79/4/2346.full. A number of other persistent RNA viruses, such as hepatitis and HIV, have evolved resistance to existing antiviral drugs. Hepatitis B Virus (lamivudine, tenofovir, entecavir): doi:10.1002/hep.22900. Hepatitis C Virus (Telaprevir, BILN2061, ITMN-191, SCH6, Boceprevir, AG-021541, ACH-806): doi:10.1002/hep.22549. HIV has many drug resistant mutations, see hivdb.stanford.edu/ for more information. Aside from drug resistance, there are a number of clinically relevant mutations that could be targeted with the CRISPR systems according to the invention as described herein. For instance, persistent versus acute infection in LCMV: doi:10.1073/pnas.1019304108; or increased infectivity of Ebola: http://doi.org/10.1016/j.cell.2016.10.014 and http://doi.org/10.1016/j.cell.2016.10.013.

In some embodiments, the methods can be used to detect SARS-CoV2 or a variant thereof in a sample, including but not limited to the B.1.1.7 (a.k.a. 20I/501Y.V1 Variant of Concern (VOC) 202012/01, BEI Reference Isolate NR-54000), B.1.351 (a.k.a. 20H/501Y.V2, BEI Reference Isolate NR-54009), P.1 (a.k.a. 20J/501Y.V3, BEI Reference Isolate NR-54982), B.1.427, B.1.429, B.1.526, B.1.525, and P.2. See e.g., Davies et al. 2021. Science. DOI: 10.1126/science.abg3055, Jangra et al., medRxiv. 2021 Jan. 29:2021.01.26.21250543. doi: 10.1101/2021.01.26.21250543; Yuan et al., bioRxiv. 2021 Feb. 17:2021.02.16.430500. doi: 10.1101/2021.02.16.430500; Long et al., Am J Pathol. 2021 Mar. 16:S0002-9440(21)00108-5. doi: 10.1016/j.ajpath.2021.03.004; Deng et al., medRxiv. 2021 Mar. 9:2021.03.07.21252647. doi: 10.1101/2021.03.07.21252647; Tchesnokova et al., bioRxiv. 2021 Mar. 11:2021.02.22.432189. doi: 10.1101/2021.02.22.432189; Starr et al., bioRxiv. 2021 Feb. 22:2021.02.17.431683. doi: 10.1101/2021.02.17.431683; Annavajhala et al., Uhlemann A C; medRxiv. 2021 Feb. 25:2021.02.23.21252259. doi: 10.1101/2021.02.23.21252259; Science Brief from the Centers for Disease Control and Prevention (CDC) on Emerging SARS-CoV-2 Variants (www.cdc.gov).

In some embodiments the SARS-CoV2 that is detected by the method or targeted by the reagents (e.g. primers) of the kits described herein is a SARS-CoV2 or SARSCoV2 variant selected from B.1.1.7, B.1.351, P.1, or a combination thereof. In some embodiments the SARS-COV2 variant comprises a D614G mutation.

### Microbe Applications

Disclosed is a method to identify microbial species, such as bacterial, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens, in a biological or environmental sample, by detecting the presence of a target nucleic acid sequence in the sample. Multiple samples can be evaluated simultaneously using the methods and systems of the invention, by employing the use of more than one effector protein, wherein each effector protein targets a specific microbial target sequence. In this way, plurality of samples can be pooled and assays can be performed identifying a sample associated with a particular subject in which a microbe is detected.

A microbe in accordance with the invention may be a pathogenic microbe or a microbe that results in food or consumable product spoilage. A pathogenic microbe may be pathogenic or otherwise undesirable to humans, animals, or plants. For human or animal purposes, a microbe may cause a disease or result in illness. Animal or veterinary applications of the present invention may identify animals infected with a microbe. For example, the methods and systems of the invention may identify companion animals with pathogens including, but not limited to, kennel cough, rabies virus, and heartworms. In other embodiments, the methods and systems of the invention may be used for parentage testing for breeding purposes. A plant microbe may result in harm or disease to a plant, reduction in yield, or alter traits such as color, taste, consistency, odor, for food or consumable contamination purposes, a microbe may adversely affect the taste, odor, color, consistency or other commercial properties of the food or consumable product. In certain example embodiments, the microbe is a bacterial species. The bacteria may be a psychotroph, a coliform, a lactic acid bacteria, or a spore-forming bacterium. In certain example embodiments, the bacteria may be any bacterial species that causes disease or illness, or otherwise results in an unwanted product or trait. Bacteria in accordance with the invention may be pathogenic to humans, animals, or plants.

Multiplex analysis of samples enables large-scale detection of samples, reducing the time and cost of analyses. However, multiplex analyses are often limited by the availability of a biological sample. In accordance with the invention, however, alternatives to multiplex analysis may be performed such that multiple effector proteins can be added to a single sample and each masking construct may be combined with a separate quencher dye. In this case, positive signals may be obtained from each quencher dye separately for multiple detection in a single sample.

Disclosed herein are methods for distinguishing between two or more species of one or more organisms in a sample. The methods are also amenable to detecting one or more species of one or more organisms in a sample.

### Microbe Detection

In some embodiments, a method for detecting microbes in samples is provided. The one or more target molecules may be mRNA, gDNA (coding or non-coding), trRNA, or rRNA comprising a target nucleotide sequence that may be used to distinguish two or more microbial species/strains from one another. Methods for enhancing ribonucleic acid hybridization are disclosed in WO 2015/085194, entitled “Enhanced Methods of Ribonucleic Acid Hybridization” which is incorporated herein by reference. The microbe-specific target may be RNA or DNA or a protein. If DNA method may further comprise the use of DNA primers that introduce a RNA polymerase promoter as described herein. If the target is a protein than the method will utilize aptamers and steps specific to protein detection described herein.

### Detection of Single Nucleotide Variants

In some embodiments, one or more identified target sequences may be detected using sequences that are specific for and bind to the target sequence as described herein. The systems and methods of the present invention can distinguish even between single nucleotide polymorphisms present among different microbial species and therefore, use of multiple primers in accordance with the invention may further expand on or improve the number of target sequences that may be used to distinguish between species. For example, in some embodiments, the one or more primers may distinguish between microbes at the species, genus, family, order, class, phylum, kingdom, or phenotype, or a combination thereof.

Detection Based on rRNA Sequences

In certain example embodiments, the devices, systems, and methods disclosed herein may be used to identify presence of a microbial species in a sample or plurality of samples. In certain example embodiments, identification may be based on ribosomal RNA sequences, including the 16S, 23S, and 5S subunits. Methods for identifying relevant rRNA sequences are disclosed in U.S. Patent Application Publication No. 2017/0029872. In certain example embodiments, primers are configured to distinguish each species by a variable region that is unique to each species or strain. Primers may also be designed to target nucleic acids that distinguish microbes at the genus, family, order, class, phylum, kingdom levels, or a combination thereof. In certain example embodiments where amplification is used, a set of amplification primers may be designed to flanking constant regions of the ribosomal RNA sequence and to generate amplicons that comprise the one or more target sequences and a unique combination of barcodes. In certain example embodiments, the primers may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv:1307.8690 [q-bio.GN].

### Screening for Drug Resistance

In certain example embodiments, the devices, systems and methods disclosed herein may be used to screen for microbial genes of interest, for example antibiotic and/or antiviral resistance genes. Primers may be designed to distinguish between known genes of interest. Samples, including clinical samples, may then be screened using the embodiments disclosed herein for detection of such genes. The ability to screen for drug resistance at POC would have tremendous benefit in selecting an appropriate treatment regime. In certain example embodiments, the antibiotic resistance genes are carbapenemases including KPC, NDM1, CTX-M15, OXA-48. Other antibiotic resistance genes are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

Ribavirin is an effective antiviral that hits a number of RNA viruses. Several clinically important viruses have evolved ribavirin resistance including Foot and Mouth Disease Virus doi:10.1128/JVI.03594-13; polio virus (Pfeifer and Kirkegaard. PNAS, 100(12):7289-7294, 2003); and hepatitis C virus (Pfeiffer and Kirkegaard, J. Virol. 79(4):2346-2355, 2005). A number of other persistent RNA viruses, such as hepatitis and HIV, have evolved resistance to existing antiviral drugs: hepatitis B virus (lamivudine, tenofovir, entecavir) doi:10/1002/hep22900; hepatitis C virus (telaprevir, BILN2061, ITMN-191, SCh6, boceprevir, AG-021541, ACH-806) doi:10.1002/hep.22549; and HIV (many drug resistance mutations) hivb.standford.edu. The embodiments disclosed herein may be used to detect such variants among others.

Aside from drug resistance, there are a number of clinically relevant mutations that could be detected with the embodiments disclosed herein, such as persistent versus acute infection in LCMV (doi:10.1073/pnas.1019304108), and increased infectivity of Ebola (Diehl et al. Cell. 2016, 167(4):1088-1098.

As described herein elsewhere, closely related microbial species (e.g. having only a single nucleotide difference in a given target sequence) may be distinguished by introduction of a synthetic mismatch in the gRNA.

In one aspect of the invention, a method comprises detecting one or more pathogens. In this manner, differentiation between infection of a subject by individual microbes may be obtained, or differentiation of subject based on infection of one or more microbes can be accomplished. In some embodiments, such differentiation may enable detection or diagnosis by a clinician of specific diseases, for example, different variants of a disease. Preferably the pathogen sequence is a genome of the pathogen or a fragment thereof. The method further may comprise determining the evolution of the pathogen. Determining the evolution of the pathogen may comprise identification of pathogen mutations, e.g. nucleotide deletion, nucleotide insertion, nucleotide substitution. Amongst the latter, there are non-synonymous, synonymous, and noncoding substitutions. Mutations are more frequently non-synonymous during an outbreak. The method may further comprise determining the substitution rate between two pathogen sequences analyzed as described above. Whether the mutations are deleterious or even adaptive would require functional analysis, however, the rate of non-synonymous mutations suggests that continued progression of this epidemic could afford an opportunity for pathogen adaptation, underscoring the need for rapid containment. Thus, the method may further comprise assessing the risk of viral adaptation, wherein the number non-synonymous mutations is determined. (Gire, et al., Science 345, 1369, 2014).

### Monitoring Outbreaks

In some embodiments, the system or methods of use thereof as described herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, contact tracing, or a mechanism involved in a disease outbreak caused by a pathogen.

The pattern of pathogen transmission may comprise continued new transmissions from the natural reservoir of the pathogen or subject-to-subject transmissions (e.g. human-to-human transmission) following a single transmission from the natural reservoir or a mixture of both. In one embodiment, the pathogen transmission may be bacterial or viral transmission, in such case, the target sequence is preferably a microbial genome or fragments thereof. In one embodiment, the pattern of the pathogen transmission is the early pattern of the pathogen transmission, i.e. at the beginning of the pathogen outbreak. Determining the pattern of the pathogen transmission at the beginning of the outbreak increases likelihood of stopping the outbreak at the earliest possible time thereby reducing the possibility of local and international dissemination.

Determining the pattern of the pathogen transmission may comprise detecting a pathogen sequence according to the methods described herein. Determining the pattern of the pathogen transmission may further comprise detecting shared intra-host variations of the pathogen sequence between the subjects and determining whether the shared intra-host variations show temporal patterns. Patterns in observed intrahost and interhost variation provide important insight about transmission and epidemiology (Gire, et al., 2014).

Detection of shared intra-host variations between the subjects that show temporal patterns is an indication of transmission links between subject (in particular between humans) because it can be explained by subject infection from multiple sources (superinfection), sample contamination recurring mutations (with or without balancing selection to reinforce mutations), or co-transmission of slightly divergent viruses that arose by mutation earlier in the transmission chain (Park, et al., Cell 161(7):1516-1526, 2015). Detection of shared intra-host variations between subjects may comprise detection of intra-host variants located at common single nucleotide polymorphism (SNP) positions. Positive detection of intra-host variants located at common (SNP) positions is indicative of superinfection and contamination as primary explanations for the intra-host variants. Superinfection and contamination can be parted on the basis of SNP frequency appearing as inter-host variants (Park, et al., 2015). Otherwise superinfection and contamination can be ruled out. In this latter case, detection of shared intra-host variations between subjects may further comprise assessing the frequencies of synonymous and nonsynonymous variants and comparing the frequency of synonymous and nonsynonymous variants to one another. A nonsynonymous mutation is a mutation that alters the amino acid of the protein, likely resulting in a biological change in the microbe that is subject to natural selection. Synonymous substitution does not alter an amino acid sequence. Equal frequency of synonymous and nonsynonymous variants is indicative of the intra-host variants evolving neutrally. If frequencies of synonymous and nonsynonymous variants are divergent, the intra-host variants are likely to be maintained by balancing selection. If frequencies of synonymous and nonsynonymous variants are low, this is indicative of recurrent mutation. If frequencies of synonymous and nonsynonymous variants are high, this is indicative of co-transmission (Park, et al., 2015).

Like Ebola virus, Lassa virus (LASV) can cause hemorrhagic fever with high case fatality rates. Andersen et al. generated a genomic catalog of almost 200 LASV sequences from clinical and rodent reservoir samples (Andersen, et al., Cell Volume 162, Issue 4, p 738-750, 13 Aug. 2015). Andersen et al. show that whereas the 2013-2015 EVD epidemic is fueled by human-to-human transmissions, LASV infections mainly result from reservoir-to-human infections. Andersen et al. elucidated the spread of LASV across West Africa and showed that this migration was accompanied by changes in LASV genome abundance, fatality rates, codon adaptation, and translational efficiency. The method may further comprise phylogenetically comparing a first pathogen sequence to a second pathogen sequence, and determining whether there is a phylogenetic link between the first and second pathogen sequences. The second pathogen sequence may be an earlier reference sequence. If there is a phylogenetic link, the method may further comprise rooting the phylogeny of the first pathogen sequence to the second pathogen sequence. Thus, it is possible to construct the lineage of the first pathogen sequence. (Park, et al., 2015).

The method may further comprise determining whether the mutations are deleterious or adaptive. Deleterious mutations are indicative of transmission-impaired viruses and dead-end infections, thus normally only present in an individual subject. Mutations unique to one individual subject are those that occur on the external branches of the phylogenetic tree, whereas internal branch mutations are those present in multiple samples (i.e. in multiple subjects). Higher rate of nonsynonymous substitution is a characteristic of external branches of the phylogenetic tree (Park, et al., 2015).

In internal branches of the phylogenetic tree, selection has had more opportunity to filter out deleterious mutants. Internal branches, by definition, have produced multiple descendent lineages and are thus less likely to include mutations with fitness costs. Thus, lower rate of nonsynonymous substitution is indicative of internal branches (Park, et al., 2015).

Synonymous mutations, which likely have less impact on fitness, occurred at more comparable frequencies on internal and external branches (Park, et al., 2015).

By analyzing the sequenced target sequence, such as viral genomes, it is possible to discover the mechanisms responsible for the severity of the epidemic episode such as during the 2014 Ebola outbreak. For example, Gire et al. made a phylogenetic comparison of the genomes of the 2014 outbreak to all 20 genomes from earlier outbreaks suggests that the 2014 West African virus likely spread from central Africa within the past decade. Rooting the phylogeny using divergence from other ebolavirus genomes was problematic (6, 13). However, rooting the tree on the oldest outbreak revealed a strong correlation between sample date and root-to-tip distance, with a substitution rate of 8×10-4 per site per year (13). This suggests that the lineages of the three most recent outbreaks all diverged from a common ancestor at roughly the same time, around 2004, which supports the hypothesis that each outbreak represents an independent zoonotic event from the same genetically diverse viral population in its natural reservoir. They also found out that the 2014 EBOV outbreak might be caused by a single transmission from the natural reservoir, followed by human-to-human transmission during the outbreak. Their results also suggested that the epidemic episode in Sierra Leon might stem from the introduction of two genetically distinct viruses from Guinea around the same time (Gire, et al., 2014).

It has been also possible to determine how the Lassa virus spread out from its origin point, in particular thanks to human-to-human transmission and even retrace the history of this spread 400 years back (Andersen, et al., Cell 162(4):738-50, 2015).

In relation to the work needed during the 2013-2015 EBOV outbreak and the difficulties encountered by the medical staff at the site of the outbreak, and more generally, the method of the invention makes it possible to carry out sequencing using fewer selected probes such that sequencing can be accelerated, thus shortening the time needed from sample taking to results procurement. Further, kits and systems can be designed to be usable on the field so that diagnostics of a patient can be readily performed without need to send or ship samples to another part of the country or the world.

In any method described above, sequencing the target sequence or fragment thereof may be used any of the sequencing processes described above. Further, sequencing the target sequence or fragment thereof may be a near-real-time sequencing. Sequencing the target sequence or fragment thereof may be carried out according to previously described methods (Experimental Procedures: Matranga et al., 2014; and Gire, et al., 2014). Sequencing the target sequence or fragment thereof may comprise parallel sequencing of a plurality of target sequences. Sequencing the target sequence or fragment thereof may comprise Illumina sequencing.

Analyzing the target sequence or fragment thereof that hybridizes to one or more of the selected probes may be an identifying analysis, wherein hybridization of a selected probe to the target sequence or a fragment thereof indicates the presence of the target sequence within the sample.

Currently, primary diagnostics are based on the symptoms a patient has. However, various diseases may share identical symptoms so that diagnostics rely much on statistics. For example, malaria triggers flu-like symptoms: headache, fever, shivering, joint pain, vomiting, hemolytic anemia, jaundice, hemoglobin in the urine, retinal damage, and convulsions. These symptoms are also common for septicemia, gastroenteritis, and viral diseases. Amongst the latter, Ebola hemorrhagic fever has the following symptoms fever, sore throat, muscular pain, headaches, vomiting, diarrhea, rash, decreased function of the liver and kidneys, internal and external hemorrhage.

When a patient is presented to a medical unit, for example in tropical Africa, basic diagnostics will conclude malaria because statistically, malaria is the most probable disease within that region of Africa. The patient is consequently treated for malaria although the patient might not actually have contracted the disease and the patient ends up not being correctly treated. This lack of correct treatment can be life-threatening especially when the disease the patient contracted presents a rapid evolution. It might be too late before the medical staff realizes that the treatment given to the patient is ineffective and comes to the correct diagnostics and administers the adequate treatment to the patient.

In other cases, a disease such as a viral infection may occur without any symptoms, or had caused symptoms but faded before the patient is presented to the medical staff. In such cases, either the patient does not seek any medical assistance or the diagnosis is complicated due to the absence of symptoms on the day of the presentation.

The present invention may also be used in concert with other methods of diagnosing disease, identifying pathogens and optimizing treatment based upon detection of nucleic acids, such as mRNA in crude, non-purified samples.

In some cases, the patient is presented to the medical staff for diagnostics of particular symptoms. The method of the invention makes it possible not only to identify which disease causes these symptoms but at the same time determine whether the patient suffers from another disease he was not aware of. This information might be of utmost importance when searching for the mechanisms of an outbreak. Indeed, groups of patients with identical viruses also show temporal patterns suggesting a subject-to-subject transmission links.

### Viruses

Primers may be used to enrich for a viral infection (e.g., of a subject or plant), including a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2.

By way of example only, several clinically important viruses have evolved ribavirin resistance including Foot and Mouth Disease Virus doi:10.1128/JVI.03594-13; polio virus (Pfeifer and Kirkegaard. PNAS, 100(12):7289-7294, 2003); and hepatitis C virus (Pfeiffer and Kirkegaard, J. Virol. 79(4):2346-2355, 2005). A number of other persistent RNA viruses, such as hepatitis and HIV, have evolved resistance to existing antiviral drugs: hepatitis B virus (lamivudine, tenofovir, entecavir) doi:10/1002/hep22900; hepatitis C virus (telaprevir, BILN2061, ITMN-191, SCh6, boceprevir, AG-021541, ACH-806) doi:10.1002/hep.22549; and HIV (many drug resistance mutations) hivb.standford.edu. The embodiments disclosed herein may be used to detect such variants among others.

In certain example embodiments, the systems, devices, and methods, disclosed herein are directed to detecting viruses in a sample. The embodiments disclosed herein may be used to detect viral infection (e.g. of a subject or plant), or determination of a viral strain, including viral strains that differ by a single nucleotide polymorphism. The virus may be a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khuj and virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or human parainfluenza virus (HPIV-1, HPIV-2, HPIV-3, HPIV-4) Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e. after infection has occurred or prophylactically, i.e. before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

In embodiments, the virus is associated with a respiratory illness. In an aspect, the virus is a coronavirus. In certain embodiments, the systems, methods and compositions comprise two or more binding molecules to one or more viruses or subtypes. Multiplex design of primer molecules for the detection of coronaviruses and/or other respiratory viruses in a sample to identify the cause of a respiratory infection is envisioned, and design can be according to the methods disclosed herein. Regarding detection of coronavirus, primer design can be predicated on genome sequences disclosed in Tian et al, “Potent binding of 2019 novel coronavirus spike protein by a SARS coronavirus-specific human monoclonal antibody”; doi: 10.1101/2020.01.28.923011, incorporated by reference, which details human monoclonal antibody, CR3022 binding of the 2019-nCoV RBD (KD of 6.3 nM) or Sequences of the 2019-nCoV are available at GISAID accession no. EPI ISL 402124 and EPI ISL 402127-402130, and described in doi:10.1101/2020.01.22.914952, or EP ISL 402119-402121 and EP ISL 402123-402124; see also GenBank Accession No. MN908947.3. Molecule design can target unique viral genomic regions of SARS-CoV-2 (also referred to as 2019-nCoV) or conserved genomic regions across one or more viruses of the coronavirus family. The coronavirus is a positive-sense single stranded RNA family of viruses, infecting a variety of animals and humans. SARS-CoV is one type of coronavirus infection, as well as MERS-CoV. In an aspect, one may use known SARS and SARS-related coronaviruses or other viruses from one or more hosts to generate a non-redundant alignment. Related viruses can be found, for example in bats.

Design can include species level Severe acute respiratory syndrome-related coronavirus species. Includes SARS-CoV-2, SARS-CoV-1, and SARS-like CoV. Gene targets may comprise ORF lab, N protein, RNA-dependent RNA polymerase (RdRP), E protein, ORF1b-nsp14, Spike glycoprotein (S), or pancorona targets. Molecular assays have been under development and can be used as a starting point to develop molecules for the methods and systems described herein. See, “Diagnostic detection of 2019-nCoV by real-time RT-PCR” Charité, Berlin Germany (17 Jan. 2020)′ Detection of 2019 novel coronavirus (2019-nCoV) in suspected human cases by RT-PCR—Hong Kong University (23 Jan. 2020); PCR and sequencing protocol for 2019-nCoV—Department of Medical Sciences, Ministry of Public Health, Thailand (updated 28 Jan. 2020); PCR and sequencing protocols for 2019-nCoV-National Institute of Infectious Diseases Japan (24 Jan. 2020); US CDC panel primer and probes—U.S. CDC, USAV—U.S. CDC, USA (28 Jan. 2020); China CDC Primers and probes for detection 2019-nCoV (24 Jan. 2020), incorporated in their entirety by reference. Further, the molecule design may exploit differences or similarities with SARS-CoV. In an example, the assay is set for subspecies-level, identifying the cause of the COVID-19 outbreak, and may exclude detection of highly related RaTG13 genome and other bat and pangolin SARS-like CoVs.

Design can include subspecies-level detection of SARS-like CoV, including most known bat and pangolin SARS-like CoVs, optionally excluding detection of SARS-CoV-2 and SARS-CoV-1. Other human coronaviruses can be detected, including for example, HCoV-229E, HCoV-HKU1, HCoV-NL63, Betacoronivirus 1. Orthomyxyxoviruses panels can also be designed, including all known subtypes of influenza A virus, segment 2; all H1 subtypes (e.g., H1N1), segment 4; all H3 subtypes (e.g., H3N2), segment 4; N1 subtypes (e.g., H1N1) segment 6; all N2 subtypes (e.g., H3N2), segment 6; or all known lineages of influenza B virus, segment 1. Similar design for paramyxoviruses, including HPIV-1, HPIV-2, HPIV-3, or FPIV-4. Design of Picornaviruses panel, including Rhinovirus, A, B, C or a combination thereof, Enterovirus, A, B, C, D or a combination thereof, Phenumoviruses, including HRSV (Human orthopneumovirus) and HMPV (Human metapneumovirus). Other coronaviruses can be detected, including in other species, such as hedgehogs, rabbits, mice, pangolin and bats. Exemplary coronaviruses can include Bat Hp-betacoronavirus Zhejiang2013, pipistrellus bat coronavirus HKU5, rabbit coronavirus HKU14, Rousettus bat coronavirus GCCDC1, Rousettus bat coronavirus HKU9, Tylonycteris bat coronavirus HKU4, coronavirus HKU15, Byulbul coronavirus HKU11, common moorhen coronavirus HKU21, murine coronavirus, China Rattus coronavirus HKU24, Rhinolpophus ferrumequinum alphacoronavirus HuB-2013, Scotophilus bat coronavirus, 512, Wencheng Sm shreq coronavirus, Rhinolophus bat coronavirus HKU2, Nyctalus velutinum alphacoronavirus SC-2103, Porcine epidemic diarrhea virus, NL63-related bat coronavirus strain BtKYNL63-9b, Myotis ricketti alphacoronavirus Sax-2011, Mink coronavirus 1, Ferret coronavirus, Miniopterus bat coronavirus HKU8, Alphacoronavirusl, BtRf-AlphaCoV/YN2012, Coronavirus AcCoV-JC34, Bat coronavirus CDPHE15, Lucheng Rn rat coronavirus, Batr coronavirus CDPHE15, Magpie-robin coronavirus HKU18, Munia coronavirus HKU13, Night heron coronavirus HKU19, Sparrow coronavirus HKU17, Thrush coronavirus HKU12, White-eye coronavirus HKU16, Wigeon Coronavirus HKU20, Avian coronavirus, Beluga whale coronavirus SW1.

Researchers have recently identified similarities and differences between 2019-nCoV and SARS-CoV. “Coronavirus Genome Annotation Reveals Amino Acid Differences with Other SARS Viruses,” genomeweb, Feb. 10, 2020. For example, molecules based on the 8a protein, which was present in SARS-CoV but absent in 2019-nCoV, can be utilized to differentiate between the viruses. Similarly, the 8b and 3b proteins have different lengths in SARS-CoV and 2019-nCoV and can be utilized to design molecules to detect non-overlapping proteins of nucleotides encoding in the two viruses. Wu et al., Genome Composition and Divergence of the Novel Coronavirus (2019-nCoV) Originating in China, Cell Host & Microbe (2020), DOI: 10.1016/j.chom.2020.02.001, incorporated herein by reference, including all supplemental information, in particular Table S1.

The binding molecules herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, or a mechanism involved in a disease outbreak caused by a pathogen. The rapid ability to design binding molecules according to the evolution of a pathogen may further identify a pattern of transmission, including, for example, superinfection, contamination, deleterious or adaptive mutations, and mechanisms responsible for the severity of an epidemic episode. Such methods of monitoring outbreaks can be as described, for example in International Publication WO 2018/107129 [0306]-[0326], incorporated herein by reference.

In some embodiments, the binding molecules herein can be used in a method described herein to determine the presence of (e.g., in a sample) and/or the evolution of variants of a pathogen. In some embodiments, such a pathogen can be SARS-CoV2 or a variant thereof, including but not limited to the B.1.1.7 (a.k.a. 20I/501Y.V1 Variant of Concern (VOC) 202012/01, BEI Reference Isolate NR-54000), B.1.351 (a.k.a. 20H/501Y.V2, BEI Reference Isolate NR-54009), P.1 (a.k.a. 20J/501Y.V3, BEI Reference Isolate NR-54982), B.1.427, B.1.429, B.1.526, B.1.525, and P.2. See e.g., Davies et al. 2021. Science. DOI: 10.1126/science.abg3055, Jangra et al., medRxiv. 2021 Jan. 29:2021.01.26.21250543. doi: 10.1101/2021.01.26.21250543; Yuan et al., bioRxiv. 2021 Feb. 17:2021.02.16.430500. doi: 10.1101/2021.02.16.430500; Long et al., Am J Pathol. 2021 Mar. 16:50002-9440(21)00108-5. doi: 10.1016/j.ajpath.2021.03.004; Deng et al., medRxiv. 2021 Mar. 9:2021.03.07.21252647. doi: 10.1101/2021.03.07.21252647; Tchesnokova et al., bioRxiv. 2021 Mar. 11:2021.02.22.432189. doi: 10.1101/2021.02.22.432189; Starr et al., bioRxiv. 2021 Feb. 22:2021.02.17.431683. doi: 10.1101/2021.02.17.431683; Annavajhala et al., Uhlemann A C; medRxiv. 2021 Feb. 25:2021.02.23.21252259. doi: 10.1101/2021.02.23.21252259; Science Brief from the Centers for Disease Control and Prevention (CDC) on Emerging SARS-CoV-2 Variants (www.cdc.gov).

The pattern of pathogen transmission may comprise continued new transmissions from the natural reservoir of the pathogen or subject-to-subject transmissions (e.g., human-to-human transmission) following a single transmission from the natural reservoir or a mixture of both. In one embodiment, the pathogen transmission may be bacterial or viral transmission, in such case, the target sequence is preferably a microbial genome or fragments thereof. In one embodiment, the pattern of the pathogen transmission is the early pattern of the pathogen transmission, i.e. at the beginning of the pathogen outbreak. Determining the pattern of the pathogen transmission at the beginning of the outbreak increases likelihood of stopping the outbreak at the earliest possible time thereby reducing the possibility of local and international dissemination.

Further embodiments are illustrated in the following Examples which are given for illustrative purposes only and are not intended to limit the scope of the invention.

### EXAMPLES

### Example 1—Design of Population-Scale Testing for SARS-CoV-2

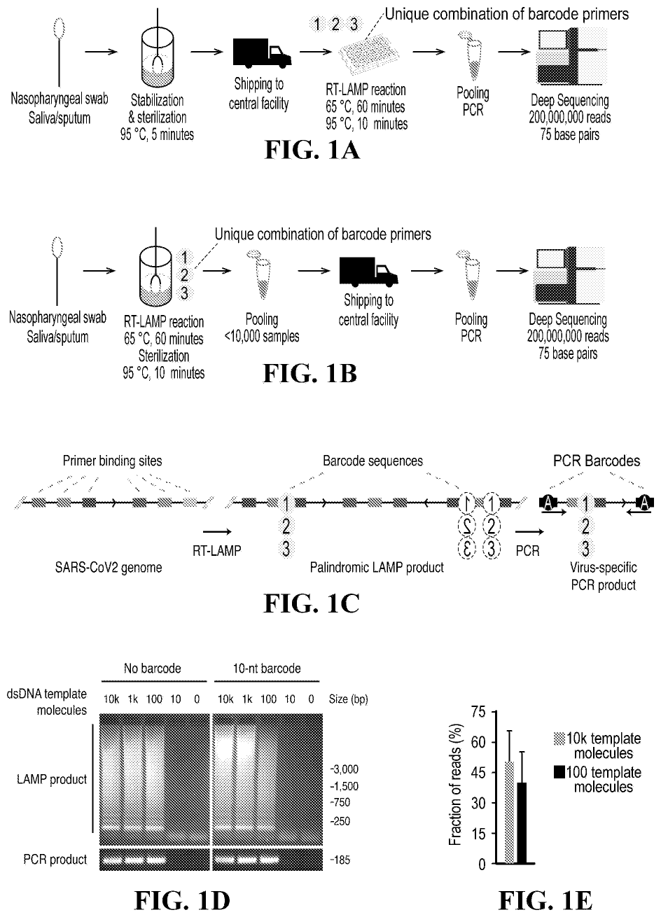

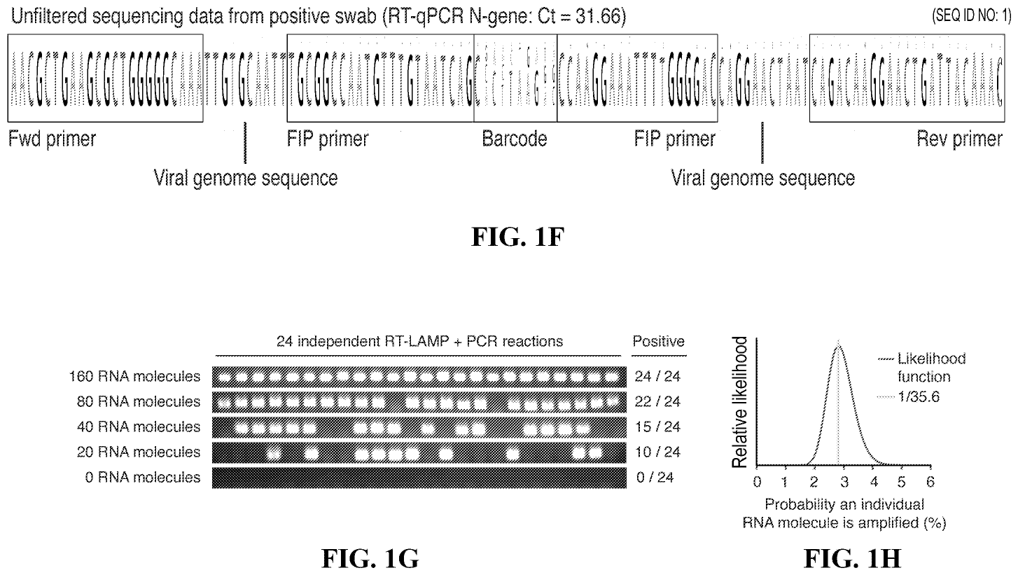

In accordance with certain example embodiments, population-scale testing for SARS-CoV-2 infection includes the following steps a barcoded RT-LAMP reaction is performed on an unpurified swab sample with primers specific for the SARS-CoV-2 genome, which is followed by large-scale pooling of samples, PCR amplification with additional barcoding, deep sequencing, and data analysis to identify positive individuals (FIG. 1A) (see below for detailed suggested protocol). RT-LAMP reactions have been demonstrated to offer high sensitivity for sequence-specific viral nucleic acid detection (Lamb et al., 2020; Yang et al., 2020; Zhang et al., 2020), even from unpurified samples (Estrela et al., 2019). To perform a barcoded RT-LAMP reaction, the unique LAMP primer design was taken advantage of: by inserting a barcode sequence into the forward inner primer (FIP) it was predicted that barcoded palindromic amplification products can be generated (FIG. 1B), allowing parallel processing of large numbers of samples after the initial amplification step. Three barcoded primer sets were designed based on validated RT-LAMP amplicons (Tables 1A-1C, (Broughton et al., 2020; Lamb et al., 2020; Zhang et al., 2020)). The chosen primers fully matched 98.3% (amplicon A), 96.9% (amplicon B), and 95.6% (amplicon C) of 183 SARS-CoV-2 genomes available in the NCBI database (as of Apr. 1, 2020). For initial validation experiments, primers for amplicon B were used. Ten-nucleotide barcodes were inserted from a set of either 1,000 or 10,000 sequences with GC content ranging from 30% to 70% and no homopolymer repeats of more than four nucleotides. Barcodes were made robust to sequence errors by ensuring a minimum Levenshtein edit distance between any barcode pair sufficient to detect either one (10,000 barcode set) or two (1,000 barcode set) insertion, deletion, or substitution errors (Tables 1A-1B; the code used is available at: https://github.comifeldman4/OpticalPooledScreensiblob/masteriops/pool_design.py).

Comparing barcoded LAMP reactions to non-barcoded controls using a dsDNA surrogate template for SARS-CoV-2 we confirmed that the presence of a 10-nt barcode within the FIP primer does not affect LAMP sensitivity, product amounts, or downstream PCR amplification (FIG. 1C). To limit the number of unique barcode primers that have to be synthesized and handled in order to test a given number of samples, a compressed barcode space can be used (FIG. 1D) (see below for details). If it is assumed that a small fraction of tested samples is expected to be positive (Gudbjartsson et al., 2020), samples can be nearly unambiguously encoded using unique combinations of barcodes out of an overlapping barcode space.

### Deployment Scenarios

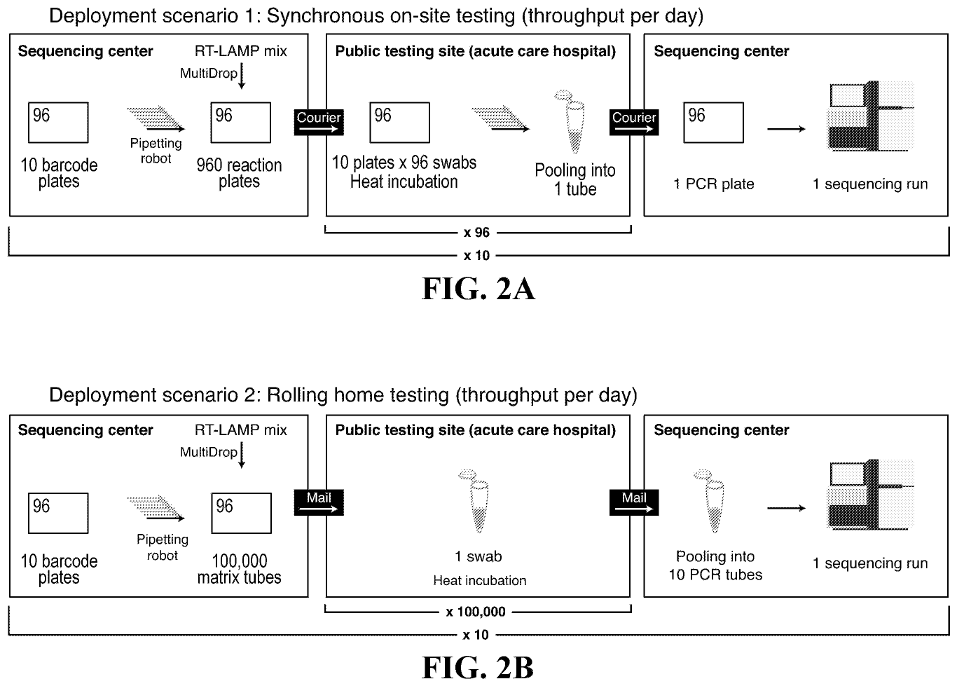

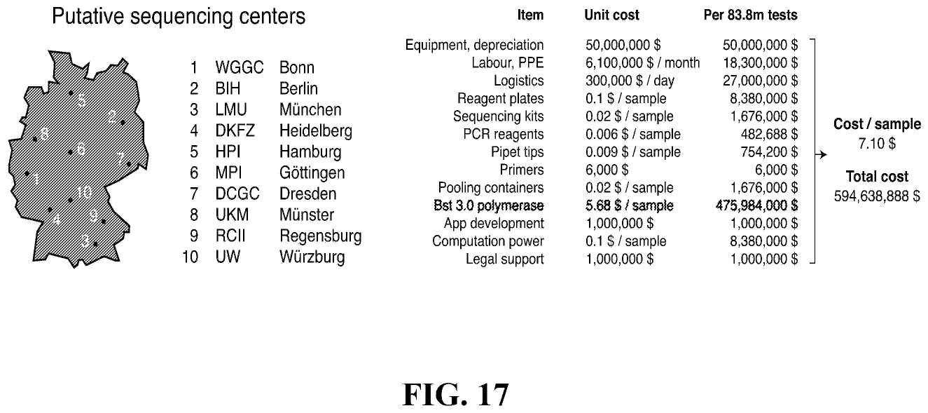

Two deployment scenarios of this method were modeled for testing a large fraction of the population of Germany (83.8 million people). The first scenario is based on the establishment of 1,000 testing sites (located at the roughly 2,000 hospitals that exist in Germany). Each testing site takes swabs from 1,000 asymptomatic people per day, performs an RT-LAMP reaction using a kit, pools all reactions from a given day, delivers the pool to one of ten sequencing centers located throughout the country, and picks up reagent kits for the next day (FIG. 2, top sequence). Each sequencing center operates one Illumina NextSeq sequencer and one Beckmann Biomek pipetting robot, infrastructure that already exists at academic facilities (FIG. 17, Note 1). Appointments and fully anonymized data reporting are provided through a smartphone app (Note 2). In this scenario, it is estimated that the cost to run a sufficient number of tests to cover the German population of 594.6 million USD (FIG. 17), which is mainly determined by the cost of Bst polymerase 3.0 (NEB); it is estimated that the total cost could be reduced to less than 170 million USD (less than 2 USD per sample) through scaled-up enzyme production, as wild-type Bst LF polymerase is commercially available for the equivalent of 0.49 USD per reaction (MCLAB). Testing a large fraction of the population would take three months in this model scenario.

The second scenario deals with long-term control of viral outbreaks. Here a decentralized testing scenario was additionally proposed where swabs are taken by local doctors or even by people at home, and are delivered to and processed by the central sequencing facilities in a rolling manner (FIG. 2, bottom sequence). This precludes pre-determined barcoding and pooling of samples to allow for efficient logistics. Based on the assumption that the fraction of virus-positive people in the population is low, group testing during barcoding is employed by using a unique combination of five out of 10,000 orthogonal barcodes per sample (10,000 choose 5=8.33×10¹⁷ unique combinations) (schematic example illustrated in FIG. 1D).

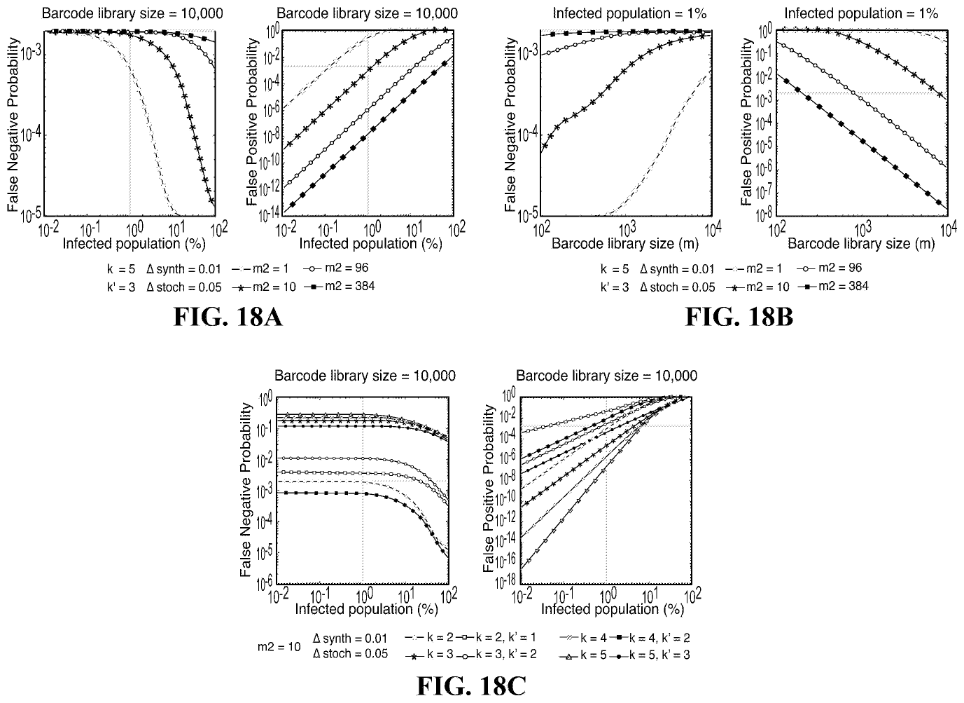

In order to assess the feasibility of using the proposed method to screen the population of Germany for bearing detectable levels of SARS-CoV-2 RNA in the upper respiratory tract, the following assumptions were made: An Illumina NextSeq sequencing run can generate 200 million sequencing reads in 14 hours. Even allowing for library skewing due to differences in viral loads over multiple orders of magnitude, this should be sufficient to sequence 100,000 samples per run, largely because the vast majority of samples will be negative. Conservatively, it was assumed that 1% of synthesized barcode primers systematically fail to work (Δ_{synth}=0.01), while additionally 5% of all sample-specific barcodes are not detected due to varying sequencing depth (Δ_{stoch}=0.05; this is independent of dropout due to low viral load). For automated assembly of testing reactions with unique barcode combinations, it was anticipated that up to m=10,000 barcode primers can be handled by available pipetting robots. Under these assumptions, Applicant investigated for 100,000 samples what number of barcode primers total (m), number of barcodes per sample (k), and number of sub-pools after the RT-LAMP reaction (m₂) would allow minimal false-positive and false-negative rates of detection (FIGS. 18A-18C). Interpreting the compressive barcoding problem as a Bloom filter, it was predicted that when using k=5 barcodes per sample, requesting k′=3 barcodes to be detected per sample, and processing m₂=10 sub-pools per run, both the false-negative and false-positive rates of detection will be less than 0.2% as long as the global frequency of positive samples is below 1.3% (an assumption consistent with early reports (Gudbjartsson et al., 2020))(FIG. 18A).

### Discussion

LAMP and RT-LAMP have been previously established for use as highly sensitive methods for pathogen detection from unpurified human samples with detection limits below 100 nucleic acid molecules. While colorimetric or turbidimetric (Mori and Notomi, 2009) readouts of LAMP reactions can suffer from false positive results (Estrela et al., 2019), it was anticipated that the proposed sequencing-based readout will only detect correct fusions of barcode sequences with two stretches of viral sequence as positive. To further increase specificity, viral sequences can be filtered for sequence portions that are non-overlapping with primer sequences. In addition, it was believed that this multiplexing-LAMP strategy is unlikely to suffer from barcode cross-contamination originating from template switching events at the PCR stage, as two template switching events would be required in order to create a sequencing-compatible amplicon.

In the outlined protocol, early pooling allows for scalable logistics. To that end, one or several barcode sequences need to be inserted in the forward inner primer (FIP) or backward inner primer (BIP) primer used during the RT-LAMP reaction, which was experimentally validated. It was considered that one limitation of the proposed approach is that skewing of sample representation at the pooling stage may affect testing sensitivity. Although in initial tests, the LAMP reaction saturated in positive samples independent of template concentration, thus potentially equalizing the representation across positive samples in an advantageous manner, the reaction might also add random skewing to pooled samples, which has to be further experimentally explored and modelled by numerical simulation. Other important parameters to consider are the compatibility of the RT-LAMP reaction with unpurified swab samples, the stability of RT-LAMP reactions during shipment.

The first deployment scenario requires low amounts of consumables (0.01 96-deep-well plate per sample, 0.1 pipet tip per sample, 0.1 μl of PCR 2× MasterMix) with the exception of Bst 3.0 polymerase (5.7 USD per sample), which, however, could be mass-produced in E. coli, titrated down, or replaced by a more cost-effective enzyme. The synthesis cost of the barcode primer library is negligible (5,000 USD total), leaving the testing infrastructure and logistics as the second-largest cost item. Once successfully validated, however, this deployment scenario could be rapidly adapted to counter future outbreaks and could be adapted in most areas of the world; furthermore, multiplexing several targets might enable scalable routine differential diagnostics.

Home testing as a second deployment scenario might offer the advantage of higher public acceptance, lower potential for viral spread at diagnostic centers, and lower probability of sample cross-contamination. However, it is not clear if the public would want and be able to engage in the medical-grade swab taking procedure (although alternatives such as gargling or perhaps even spitting may provide viable samples). In addition, the RT-LAMP reaction requires a broadly available defined heat source, which hypothetically could be a kitchen oven, a cooling down glass of boiling water, a USB-powered heating device, or a chemical reaction. Of note, the temperature ramp in an oven between 45° C. and 72° C. might allow Bst 3.0 polymerase to be largely active for several minutes, and might offer the added benefit of sterilizing closed sample containers before shipment.

It is predicted that the sequencing-based approach described here can allow scalable logistics by utilizing early-stage barcoding, while providing a low false-positive rate.

### Example Protocol

1. A fresh swab sample is inserted into a 500 μl RT-LAMP reaction, containing the following components:
   a. 1× Isothermal Amplification buffer (NEB),
   b. 6 mM MgSO₄,
   c. 1.4 mM dNTP mix,
   d. 1 μl Triton X-100 (amount to be optimized),
   e.

1.6 μM total of one or a unique set of five barcoded forward inner primer
(FIP) primers
(B-FIP-Barcode, TCTGGCCCAGTTCCTAGGTAGTNNNNNNNNNNCCAGACGAATTCGTGG
TGG (SEQ ID NO: 47)),

where Ns denote a specific barcode sequence,
f.

1.6 μM backward inner primer (BIP) prime
(B-BIP, AGACGGCATCATATGGGTTGCACGGGTGCCAATGTGATCT
(SEQ ID NO: 48)),

g.

0.2 μM B-F3 primer
(TGGCTACTACCGAAGAGCT (SEQ ID NO: 49)),

h.

0.2 μM B-B3 primer
(TGCAGCATTGTTAGCAGGAT (SEQ ID NO: 50)),

i.

0.4 μM B-LF primer
(GGACTGAGATCTTTCATTTTACCGT (SEQ ID NO: 51)),

j.

0.4 μM B-LB primer
(ACTGAGGGAGCCTTGAATACA (SEQ ID NO: 52)),

k. 160 units Bst 3.0 DNA polymerase (NEB, enzyme dilution to be optimized),
l. a dilute control template DNA or RNA differing from the target viral sequence, but sharing all primer binding sites,
a. water.
2. The RT-LAMP reaction with the swab is heated to 65° C. for 30 minutes to react, and to 95° C. for 10 minutes to sterilize.
3. Up to 100,000 reactions are pooled in batches of 1,000 or 10,000 samples per batch.
4. For each pool, a 12-cycle 100 μl PCR reaction is performed:
   a. 50 μl NEBNext 2× Master Mix (NEB),
   b.

0.5 μM PCR-B-fwd-10 primer
(ACACTCTTTCCCTACACGACGCTCTTCCGATCTCCGTCTTTGTTAGCA
CCATAGGG (SEQ ID NO: 53)),

c.

0.5 μM PCR-B-rev-12 primer
(TGACTGGAGTTCAGACGTGTGCTCTTCCGATCTACCATCTTGGACTGA
GATCTTTC (SEQ ID NO: 54)),

d. 10 μl of pooled RT-LAMP reactions pre-diluted 100-fold in water,
e. water.
5. For each pool, a secondary 12-cycle 100 μl PCR reaction is performed with:
   a. 50 μl NEBNext 2× Master Mix (NEB),
   b.

0.5 μM pool-specific fwd barcoding primer
(AATGATACGGCGACCACCGAGATCTACACNNNNNNNNNNACACTCTT
TCCCTACACGACGCT (SEQ ID NO: 55)),

where Ns denote a specific barcode sequence,
c.

0.5 μM pool-specific rev barcoding primer
(CAAGCAGAAGACGGCATACGAGATNNNNNNNNNNGTGACTGGAGTT
CAGACGTGTGCT (SEQ ID NO: 56)),

where Ns denote a specific barcode sequence,
d. 5 μl of previous PCR reaction,
e. water.
6. The PCR products are pooled, gel-purified, and sequenced on an Illumina NextSeq sequencer, or similar device.
7. Computationally, the barcodes co-occurring with the correct viral genome sequence excluding sequence portions covered by primers are determined.
8. Positive patient samples are determined by either one (single barcoding scenario) or at least three out of all five sample barcodes (compressed barcode space) being positive.

TABLE 1A
Primer Sequences for Amplicon A (Lamb et al.).

| Name | Primer Sequence (5′-3′) |
|---|---|
| A-F3 | TCCAGATGAGGATGAAGAAGA (SEQ ID NO: 57) |
| A-B3 | AGTCTGAACAACTGGTGTAAG (SEQ ID NO: 58) |
| A-FIP- | AGAGCAGCAGAAGTGGCACNNNNNNNNNNAGGTGATTGTGAAGAAGAAGAG |
| Barcode | (SEQ ID NO: 59) |
| A-BIP | TCAACCTGAAGAAGAGCAAGAA CTGATTGTCCTCACTGCC (SEQ ID NO: 60) |
| A-LF | CTCATATTGAGTTGATGGCTCA (SEQ ID NO: 61) |
| A-LB | ACAAACTGTTGGTCAACAAGAC (SEQ ID NO: 62) |

TABLE 1B
Primer Sequences for Amplicon B (Zhang et al.).

| Name | Primer Sequence (5′-3′) |
|---|---|
| B-F3 | TGGCTACTACCGAAGAGCT (SEQ ID NO: 63) |
| B-B3 | TGCAGCATTGTTAGCAGGAT (SEQ ID NO: 64) |
| B-FIP- | TCTGGCCCAGTTCCTAGGTAGTNNNNNNNNNNCCAGACGAATTC |
| Barcode | GTGGTGG (SEQ ID NO: 65) |
| B-BIP | AGACGGCATCATATGGGTTGCA CGGGTGCCAATGTGATCT (SEQ |
| | ID NO: 66) |
| B-LF | GGACTGAGATCTTTCATTTTACCGT (SEQ ID NO: 67) |
| B-LB | ACTGAGGGAGCCTTGAATACA (SEQ ID NO: 68) |
| miseq_B_ | ACACTCTTTCCCTACACGACGCTCTTCCGATCTCCGTCTTTGTTAG |
| fwd_10 | CACCATAGGG (SEQ ID NO: 69) |
| miseq_B_ | TGACTGGAGTTCAGACGTGTGCTCTTCCGATCTACCATCTTGGAC |
| rev_12 | TGAGATCTTTC (SEQ ID NO: 70) |

TABLE 1C
Primer Sequences for Amplicon C (Broughton et al.)

| Name | Primer Sequence (5′-3′) |
|---|---|
| C-F3 | AACACAAGCTTTCGGCAG (SEQ ID NO: 71) |
| C-B3 | GAAATTTGGATCTTTGTCATCC (SEQ ID NO: 72) |
| C-FIP- | TGCGGCCAATGTTTGTAATCAGNNNNNNNNNNCCAAGGAAATTT |
| Barcode | TGGGGAC (SEQ ID NO: 73) |
| C-BIP | CGCATTGGCATGGAAGTCAC TTTGATGGCACCTGTGTAG (SEQ ID |
| | NO: 74) |
| C-LF | TTCCTTGTCTGATTAGTTC (SEQ ID NO: 75) |
| C-LB | ACCTTCGGGAACGTGGTT (SEQ ID NO: 76) |
| miseq_C_ | ACACTCTTTCCCTACACGACGCTCTTCCGATCTAACGCTGAAGCG |
| fwd_10 | CTGGGGGCAAA (SEQ ID NO: 77) |
| miseq_C_ | TGACTGGAGTTCAGACGTGTGCTCTTCCGATCTGTTTGTAATCAGT |
| rev_11 | TCCTTGTCTG (SEQ ID NO: 78) |

Table 2A. 10,000 Barcodes—See Electronic File Table 2A filed in U.S. Provisional Application Ser. 63/004,456, which is incorporated by reference as if expressed herein in its entirety. See also Supplementary Table 1 of Schmid-Burgk et al., 2020, “LAMP-Seq: Population-Scale COVID-19 Diagnostics Using Combinatorial Barcoding” doi: https://doi.org/10.1101/2020.04.06.025635.
Table 2B. 1,000 Barcodes—See Electronic File Table 2B filed in U.S. Provisional Application Ser. 63/004,456, which is incorporated by reference as if expressed herein in its entirety. See also Supplementary Table 1 of Schmid-Burgk et al., 2020, “LAMP-Seq: Population-Scale COVID-19 Diagnostics Using Combinatorial Barcoding” doi: https://doi.org/10.1101/2020.04.06.025635.

TABLE 3
Complementarity Summary

| | | Primer | | | | | | |
|---|---|---|---|---|---|---|---|---|
| Primer | Fre- | Name | | | Primer | | | |
| Name | quency | Comple- | Direction | | Binding | | | |
| Comple- | in | mentary | for | | Site in SARS | | | Full |
| to SARS | SARS- | to | Binding to | | CoV-2 | Primer | Full | Primer |
| CoV-2 | CoV-2 | SARS CoV- | SARS CoV-2 | Primer | Genome | Sequence | Primer | Sequence |
| Genome Set | genomes | 2 Genome | Genome | Set | (5′-3′) | (5′-3′) | Name | (5′-3′) |
| A-F3 | 182 | F3 | For | A | TCCAGATGA | TCCAG | A-F3 | TCCAGA |
| | | | | | GGATGAAG | ATGAG | | TGAGGA |
| | | | | | AAGA (SEQ | GATGA | | TGAAGA |
| | | | | | ID NO: 79) | AGAAG | | AGA |
| | | | | | | A (SEQ | | (SEQ ID |
| | | | | | | ID NO: | | NO: 81) |
| | | | | | | 80) | | |
| A-B3 | 183 | B3 | Rev | A | CTTACACCA | AGTCTG | A-B3 | AGTCTG |
| | | | | | GTTGTTCAG | AACAA | | AACAAC |
| | | | | | ACT (SEQ ID | CTGGTG | | TGGTGT |
| | | | | | NO: 82) | TAAG | | AAG |
| | | | | | | (SEQ ID | | (SEQ ID |
| | | | | | | NO: 83) | | NO: 84) |
| A-F2 | 182 | F2 | For | A | AGGTGATTG | AGGTG | A-FIP- | AGAGCA |
| | | | | | TGAAGAAG | ATTGTG | Barcode | GCAGAA |
| | | | | | AAGAG (SEQ | AAGAA | | GTGGCA |
| | | | | | ID NO: 84) | GAAGA | | CNNNNN |
| | | | | | | G(SEQ | | NNNNNA |
| | | | | | | ID NO: | | GGTGAT |
| | | | | | | 86) | | TGTGAA |
| | | | | | | | | GAAGAA |
| | | | | | | | | GAG |
| | | | | | | | | (SEQ ID |
| | | | | | | | | NO: 87) |
| A-B1C | 180 | B1C | For | A | TCAACCTGA | TCAACC | A-BIP | TCAACC |
| | | | | | AGAAGAGC | TGAAG | | TGAAGA |
| | | | | | AAGAA (SEQ | AAGAG | | AGAGCA |
| | | | | | ID NO: 88) | CAAGA | | AGAA |
| | | | | | | A (SEQ | | CTGATT |
| | | | | | | ID NO: | | GTCCTC |
| | | | | | | 89) | | ACTGCC |
| | | | | | | | | (SEQ ID |
| | | | | | | | | NO: 90) |
| A-B2 | 181 | B2 | Rev | A | GGCAGTGA | CTGATT | A-BIP | TCAACC |
| | | | | | GGACAATC | GTCCTC | | TGAAGA |
| | | | | | AG (SEQ ID | ACTGCC | | AGAGCA |
| | | | | | NO: 91) | (SEQ ID | | AGAA |
| | | | | | | NO: 92) | | CTGATT |
| | | | | | | | | GTCCTC |
| | | | | | | | | ACTGCC |
| | | | | | | | | (SEQ ID |
| | | | | | | | | NO: 93) |
| A-LF | 180 | LF | Rev | A | TGAGCCATC | CTCATA | A-LF | CTCATA |
| | | | | | AACTCAATA | TTGAGT | | TTGAGT |
| | | | | | TGAG (SEQ | TGATG | | TGATGG |
| | | | | | ID NO: 94) | GCTCA | | CTCA |
| | | | | | | (SEQ ID | | (SEQ ID |
| | | | | | | NO: 95) | | NO: 96) |
| A-LB | 182 | LB | For | A | ACAAACTGT | ACAAA | A-LB | ACAAAC |
| | | | | | TGGTCAACA | CTGTTG | | TGTTGG |
| | | | | | AGAC (SEQ | GTCAA | | TCAACA |
| | | | | | ID NO: 97) | CAAGA | | AGAC |
| | | | | | | C (SEQ | | (SEQ ID |
| | | | | | | ID NO: | | NO: 99) |
| | | | | | | 98) | | |
| A-PCR-rev- | 182 | PCR-rev- | Rev | A | GGTACTGAA | CCTTGG | miseq_ | ACACTC |
| 10-B1C | | 10-B1C | | | GATGATTAC | TAATCA | A_fwd 10 | TTTCCCT |
| | | | | | CAAGG (SEQ | TCTTCA | | ACACGA |
| | | | | | ID NO: 100) | GTACC | | CGCTCTT |
| | | | | | | (SEQ ID | | CCGATC |
| | | | | | | NO: 101) | | T |
| | | | | | | | | CCTTGG |
| | | | | | | | | TAATCA |
| | | | | | | | | TCTTCA |
| | | | | | | | | GTACC |
| | | | | | | | | (SEQ ID |
| | | | | | | | | NO: 102) |
| A-PCR-rev- | 181 | PCR-rev- | Rev | A | AGGACAAT | AGGAC | miseq_ | TGACTG |
| 10-B2 | | 10-B2 | | | CAG (SEQ ID | AATCA | A_rev_12 | GAGTTC |
| | | | | | NO: 103) | G(SEQ | | AGACGT |
| | | | | | | ID NO: | | GTGCTC |
| | | | | | | 104) | | TTCCGA |
| | | | | | | | | TCT |
| | | | | | | | | AGGACA |
| | | | | | | | | ATCAG |
| | | | | | | | | TTCTTGC |
| | | | | | | | | TCTTC |
| | | | | | | | | (SEQ ID |
| | | | | | | | | NO: 105) |
| A-PCR-rev- | 182 | PCR-rev- | Rev | A | GAAGAGCA | TTCTTG | miseq_ | TGACTG |
| 10 | | 10 | | | AGAA (SEQ | CTCTTC | A_rev 12 | GAGTTC |
| | | | | | ID NO: 106) | (SEQ ID | | AGACGT |
| | | | | | | NO: 107) | | GTGCTC |
| | | | | | | | | TTCCGA |
| | | | | | | | | TCT |
| | | | | | | | | AGGACA |
| | | | | | | | | ATCAG |
| | | | | | | | | TTCTTGC |
| | | | | | | | | TCTTC |
| | | | | | | | | (SEQ ID |
| | | | | | | | | NO: 108) |
| B-F3 | 182 | F3 | For | B | TGGCTACTA | TGGCTA | B-F3 | TGGCTA |
| | | | | | CCGAAGAG | CTACCG | | CTACCG |
| | | | | | CT (SEQ ID | AAGAG | | AAGAGC |
| | | | | | NO: 109) | CT (SEQ | | T (SEQ ID |
| | | | | | | ID NO: | | NO: 111) |
| | | | | | | 110) | | |
| B-B3 | 183 | B3 | Rev | B | ATCCTGCTA | TGCAG | B-B3 | TGCAGC |
| | | | | | ACAATGCTG | CATTGT | | ATTGTT |
| | | | | | CA (SEQ ID | TAGCA | | AGCAGG |
| | | | | | NO: 112) | GGAT | | AT (SEQ |
| | | | | | | (SEQ ID | | ID NO: |
| | | | | | | NO: 113) | | 114) |
| B-F2 | 183 | F2 | For | B | CCAGACGA | CCAGA | B-FIP | TCTGGC |
| | | | | | ATTCGTGGT | CGAATT | | CCAGTT |
| | | | | | GG (SEQ ID | CGTGGT | | CCTAGG |
| | | | | | NO: 115) | GG (SEQ | | TAGTNN |
| | | | | | | ID NO: | | NNNNNN |
| | | | | | | 116) | | NNCCAG |
| | | | | | | | | ACGAAT |
| | | | | | | | | TCGTGG |
| | | | | | | | | TGG (SEQ |
| | | | | | | | | ID NO: |
| | | | | | | | | 117) |
| B-B1C | 177 | B1C | For | B | AGACGGCA | AGACG | B-BIP | AGACGG |
| | | | | | TCATATGGG | GCATC | | CATCAT |
| | | | | | TTGCA (SEQ | ATATG | | ATGGGT |
| | | | | | ID NO: 118) | GGTTGC | | TGCA |
| | | | | | | A (SEQ | | CGGGTG |
| | | | | | | ID NO: | | CCAATG |
| | | | | | | 119) | | TGATCT |
| | | | | | | | | (SEQ ID |
| | | | | | | | | NO: 120) |
| B-B2 | 183 | B2 | Rev | B | AGATCACAT | CGGGT | B-BIP | AGACGG |
| | | | | | TGGCACCCG | GCCAA | | CATCAT |
| | | | | | (SEQ ID NO: | TGTGAT | | ATGGGT |
| | | | | | 121) | CT (SEQ | | TGCA |
| | | | | | | ID NO: | | CGGGTG |
| | | | | | | 122) | | CCAATG |
| | | | | | | | | TGATCT |
| | | | | | | | | (SEQ ID |
| | | | | | | | | NO: 123) |
| B-LF | 183 | LF | Rev | B | ACGGTAAA | GGACT | B-LF | GGACTG |
| | | | | | ATGAAAGA | GAGAT | | AGATCT |
| | | | | | TCTCAGTCC | CTTTCA | | TTCATTT |
| | | | | | (SEQ ID NO: | TTTTAC | | TACCGT |
| | | | | | 124) | CGT | | (SEQ ID |
| | | | | | | (SEQ ID | | NO: 126) |
| | | | | | | NO: 125) | | |
| B-LB | 183 | LB | For | B | ACTGAGGG | ACTGA | B-LB | ACTGAG |
| | | | | | AGCCTTGAA | GGGAG | | GGAGCC |
| | | | | | TACA (SEQ | CCTTGA | | TTGAAT |
| | | | | | ID NO: 127) | ATACA | | ACA |
| | | | | | | (SEQ ID | | (SEQ ID |
| | | | | | | NO: 128) | | NO: 129) |
| B-PCR- | 177 | PCR- | Rev | B | CCCTATGGT | CCGTCT | miseq_ | ACACTC |
| fwd 10 | | fwd 10 | | | GCTAACAA | TTGTTA | B_fwd 10 | TTTCCCT |
| | | | | | AGACGG | GCACC | | ACACGA |
| | | | | | (SEQ ID NO: | ATAGG | | CGCTCTT |
| | | | | | 130) | G(SEQ | | CCGATC |
| | | | | | | ID NO: | | T |
| | | | | | | 131) | | CCGTCTT |
| | | | | | | | | TGTTAG |
| | | | | | | | | CACCAT |
| | | | | | | | | AGGG |
| | | | | | | | | (SEQ ID |
| | | | | | | | | NO: 132) |
| B-PCR- | 183 | PCR- | Rev | B | GAAAGATCT | ACCATC | miseq_ | TGACTG |
| rev 12 | | rev 12 | | | CAGTCCAAG | TTGGAC | B_rev 12 | GAGTTC |
| | | | | | ATGGT (SEQ | TGAGA | | AGACGT |
| | | | | | ID NO: 133) | TCTTTC | | GTGCTC |
| | | | | | | (SEQ ID | | TTCCGA |
| | | | | | | NO: 134) | | TCT |
| | | | | | | | | ACCATC |
| | | | | | | | | TTGGAC |
| | | | | | | | | TGAGAT |
| | | | | | | | | CTTTC |
| | | | | | | | | (SEQ ID |
| | | | | | | | | NO: 135) |
| C-F3 | 179 | F3 | For | C | AACACAAG | AACAC | C-F3 | AACACA |
| | | | | | CTTTCGGCA | AAGCTT | | AGCTTT |
| | | | | | G (SEQ ID | TCGGC | | CGGCAG |
| | | | | | NO: 136) | AG (SEQ | | (SEQ ID |
| | | | | | | ID NO: | | NO: 138) |
| | | | | | | 137) | | |
| C-B3 | 180 | B3 | Rev | C | GGATGACA | GAAAT | C-B3 | GAAATT |
| | | | | | AAGATCCA | TTGGAT | | TGGATC |
| | | | | | AATTTC | CTTTGT | | TTTGTCA |
| | | | | | (SEQ ID NO: | CATCC | | TCC (SEQ |
| | | | | | 139) | (SEQ ID | | ID NO: |
| | | | | | | NO: 140) | | 141) |
| C-F2 | 183 | F2 | For | C | CCAAGGAA | CCAAG | C-FIP | TGCGGC |
| | | | | | ATTTTGGGG | GAAAT | | CAATGT |
| | | | | | AC (SEQ ID | TTTGGG | | TTGTAA |
| | | | | | NO: 142) | GAC | | TCAGNN |
| | | | | | | (SEQ ID | | NNNNNN |
| | | | | | | NO: 143) | | NNCCAA |
| | | | | | | | | GGAAAT |
| | | | | | | | | TTTGGG |
| | | | | | | | | GAC |
| | | | | | | | | (SEQ ID |
| | | | | | | | | NO: 144) |
| C-B1C | 182 | B1C | For | C | CGCATTGGC | CGCATT | C-BIP | CGCATT |
| | | | | | ATGGAAGTC | GGCAT | | GGCATG |
| | | | | | AC (SEQ ID | GGAAG | | GAAGTC |
| | | | | | NO: 145) | TCAC | | AC |
| | | | | | | (SEQ ID | | TTTGAT |
| | | | | | | NO: 146) | | GGCACC |
| | | | | | | | | TGTGTA |
| | | | | | | | | G (SEQ ID |
| | | | | | | | | NO: 147) |
| C-B2 | 183 | B2 | Rev | C | CTACACAGG | TTTGAT | C-BIP | CGCATT |
| | | | | | TGCCATCAA | GGCAC | | GGCATG |
| | | | | | A (SEQ ID | CTGTGT | | GAAGTC |
| | | | | | NO: 148) | AG (SEQ | | AC |
| | | | | | | ID NO: | | TTTGAT |
| | | | | | | 149) | | GGCACC |
| | | | | | | | | TGTGTA |
| | | | | | | | | G (SEQ ID |
| | | | | | | | | NO: 150) |
| C-LF | 183 | LF | Rev | C | GAACTAATC | TTCCTT | C-LF | TTCCTTG |
| | | | | | AGACAAGG | GTCTGA | | TCTGATT |
| | | | | | AA (SEQ ID | TTAGTT | | AGTTC |
| | | | | | NO: 151) | C (SEQ | | (SEQ ID |
| | | | | | | ID NO: | | NO: 153) |
| | | | | | | 152) | | |
| C-LB | 182 | LB | For | C | ACCTTCGGG | ACCTTC | C-LB | ACCTTC |
| | | | | | AACGTGGTT | GGGAA | | GGGAAC |
| | | | | | (SEQ ID NO: | CGTGGT | | GTGGTT |
| | | | | | 154) | T (SEQ | | (SEQ ID |
| | | | | | | ID NO: | | NO: 156) |
| | | | | | | 155) | | |
| C-PCR- | 183 | PCR- | Rev | C | TTTGCCCCC | AACGC | miseq_ | ACACTC |
| fwd 10 | | fwd 10 | | | AGCGCTTCA | TGAAG | C_fwd 10 | TTTCCCT |
| | | | | | GCGTT(SEQ | CGCTG | | ACACGA |
| | | | | | ID NO: 157) | GGGGC | | CGCTCTT |
| | | | | | | AAA | | CCGATC |
| | | | | | | (SEQ ID | | T |
| | | | | | | NO: 158) | | AACGCT |
| | | | | | | | | GAAGCG |
| | | | | | | | | CTGGGG |
| | | | | | | | | GCAAA |
| | | | | | | | | (SEQ ID |
| | | | | | | | | NO: 159) |
| C-PCR- | 183 | PCR- | Rev | C | CAGACAAG | GTTTGT | miseq_ | TGACTG |
| rev 11 | | rev 11 | | | GAACTGATT | AATCA | C_rev 11 | GAGTTC |
| | | | | | ACAAAC | GTTCCT | | AGACGT |
| | | | | | (SEQ ID NO: | TGTCTG | | GTGCTC |
| | | | | | 160) | (SEQ ID | | TTCCGA |
| | | | | | | NO: 161) | | TCT |
| | | | | | | | | GTTTGT |
| | | | | | | | | AATCAG |
| | | | | | | | | TTCCTTG |
| | | | | | | | | TCTG |
| | | | | | | | | (SEQ ID |
| | | | | | | | | NO: 162) |

### Note 1

All steps below are performed by one sequencing center or one testing center per one day. A total of ten sequencing centers and 1,000 testing centers are predicted to operate in the whole country. According to below protocols, each sequencing and testing center would be staffed by two operators.

### Step 1: Preparing Reagent Plates (at Sequencing Center)

Using a MultiDrop reagent dispenser, 495 μl of RT-LAMP mastermix lacking template and FIP primer are dispensed into 960 96-well deep-well plates (“reagent plates”). Plates are stored in a cold room.

Using a Beckmann Biomek robot, for each of 10 barcode source plates (A-J):
1. An operator places the source plate on the robot deck and unseals it,
2. An operator places 96 reagent plates in the robot's hotel (alternative: process plates in smaller batches),
3. An operator places 8 96-well tip boxes in the robot's hotel,
4. In sets of 12 reagent plates (to be performed 8 times per source plate):
   i. The robot retrieves 12 reagent plates from the hotel and places them on the deck,
   ii. Using the 96-well head, the robot aspirates 65 μl of primer solution per well from the source plate,
   iii. Without changing tips, the robot dispenses 5 μl of primer solution into each of 12 reagent plates present on the deck,
   iv. The robot discards the tips,
   v. The robot returns the 12 reagent plates to the hotel.
5. The operator removes the 96 reagent plates from the hotel, seals them, and stores them for shipping in a cold room.

A courier delivers 10 reagent plates with orthogonal barcodes A-J to each testing center, located within a distance of 300 km from the sequencing center. A total of 96 testing centers are served by each sequencing center.

### Step 2: Processing Swab Samples (at Testing Center)

For each of 10 reagent plates:
1. The plate is unsealed and placed in a sterile hood, preferentially on a cooling device,
2. Swab samples are taken by a medical worker using personal protective equipment from individuals at a rate of roughly two samples/minute,
3. Swabs are inserted in successive order into the wells of the reagent plate,
4. The last two wells of every plate are left empty/spiked in with a positive control RNA,
5. When the plate is complete, it, is sealed using an adhesive foil, and incubated in the sterile hood in two ovens (20 minutes 65° C., 10 minutes 95° C.),
6. The plate is stored at room temperature.

At the end of the day or when all plates have been processed, an operator unseals all plates in a sterile hood, and pours them into a plastic container.

The combined liquid in the container is mixed, and a small sample is transferred to a 2 ml plastic screw cap tube.

A courier delivers the tube to the serving sequencing center.

### Step 3: Processing and Sequencing Pooled Samples (at Sequencing Center)

An operator gathers 96 pool tubes from testing centers in a rack, and dispenses 100 μl of each pool into a 96-well plate with well positions according to the testing center ID.

The operator prepares a PCR plate with 95 μl mastermix per well (water, 2× NEBNext mastermix, two primers).

The operator stamps over 5 μl of each template into the PCR plate using a 12-channel pipet.

The operator runs the PCR plate in a 96-well temperature cycler according to the protocol.

The operator unseals the plate.

The operator prepares a secondary PCR plate with 95 μl mastermix per well (water, 2× NEBNext mastermix, no primers).

The operator stamps in 95 unique primer combinations from a stock plate using a 12-channel pipet.

The operator stamps over 5 μl from the first PCR into the secondary PCR plate using a 12-channel pipet.

The operator runs the PCR plate in a 96-well temperature cycler according to the protocol.

On ice, the operator pools 20 μl of each of 96 secondary PCR reactions in a reservoir.

The operator runs the pool on a 2% EX E-Gel (10 pockets×10 μl pool per pocket).

The operator cuts the bands of appropriate size from the gel, and purifies the DNA using a Qiagen agarose gel purification kit according to the manufacturer's instructions.

The operator re-purifies the DNA using a Qiagen PCR purification kit according to the manufacturer's instructions.

The operator quantifies the DNA using a NanoDrop photospectrometer or alternative device.

The operator thaws a NextSeq High-Output 75-cycle kit (Illumina), inserts the DNA, and initiates a run, which takes 14 hours.

### Step 4: Data Analysis

An operator transfers the NextSeq data to a server computer.

The operator initiates barcode deconvolution using a predefined SampleSheet and the bcl2fastq tool (Illumina), which takes 2 hours.

The operator inflates the compressed output files on the server, resulting in FASTQ files of about 50 GB in total size, which takes 1 hour.

The operator executes a software program that takes in 96 FASTQ files, and saves a list of positive/negative/unresolved sample barcodes, which takes 4 hours.

The operator copies the list to an online server, and executes a software program that re-formats the data, links it to user IDs potentially gathered as described in Suppl. Note 2, and adds the results to a database, suited for anonymous web-based retrieval of testing results based on user ID (Note 2).

### Note 2. Proposed Design of a Privacy-Aware Web App for Delivering Diagnostic Results

### Step 1: Creating a Personal ID

The user opens a specific website on a computer,

The website displays a high-complexity pseudo-random QR code,

The user prints the QR code, which serves as an anonymous user ID,

If the user has no ability to print, a family member, nurse, or social worker can supply the user with a printed ID code.

### Step 2: Making an Appointment for a Swab

The user uses the camera of a smartphone to scan the printed ID code,

It opens a link to a website, which displays an available appointment slot of 15 minutes and a location,

Optionally, the user's GPS coordinates or randomly blurred GPS coordinates are used to spatially balance the population tested,

The user either reserves the slot, or requests an alternative slot.

### Step 3: Getting a Swab Taken

The user waits in line with 2 m distancing from others,

A medical worker scans the user's ID code as well as the QR code of a reagent plate,

A swab is taken, inserted into the reagent plate, and processed further.

### Step 4: Sequencing Data Analysis

Sequencing data are analyzed and stored on a server, linked to the user's ID code,

No personal information or means of identification are recorded.

### Step 5: Retrieving the Result

The user uses the camera of a smartphone to scan the printed ID code,

It opens a link to a website, which displays the test result along with further instructions,

According to law, the user might be obligated to report a positive result to a public health authority.

### REFERENCES FOR EXAMPLE 1

Broughton, J. P., Deng, X., Yu, G., Fasching, C. L., Singh, J., Streithorst, J., Granados, A., Sotomayor-Gonzalez, A., Zorn, K., Gopez, A., et al. (2020). Rapid Detection of 2019 Novel Coronavirus SARS-CoV-2 Using a CRISPR-based DETECTR Lateral Flow Assay. 1-27.
Chinazzi, M., Davis, J. T, Ajelli, M., Gioannini, C., Litvinova, M., Merler, S., Pastore y Piontti, A., Mu, K., Rossi, L., Sun, K., et al. (2020). The effect of travel restrictions on the spread of the 2019 novel coronavirus (COVID-19) outbreak. Science eaba9757-12.
Estrela, P. F. N., de Melo Mendes, G., de Oliveira, K. G., Bailão, A. M., de Almeida Soares, C. M., Assunçáo, N. A., and Duarte, G. R. M. (2019). Ten-minute direct detection of Zika virus in serum samples by RT-LAMP. Journal of Virological Methods 271, 113675.
Gudbjartsson, D. F., Helgason, A., Jonsson, H., Magnusson, O. T., Melsted, P., Norddahl, G. L., Saemundsdottir, J., Sigurdsson, A., Sulem, P., Agustsdottir, A. B., et al. (2020). Early Spread of SARS-Cov-2 in the Icelandic Population. 1-34.
Kissler, S. M., Tedijanto, C., Lipsitch, M., and Grad, Y. (2020). Social distancing strategies for curbing the COVID-19 epidemic. 1-21.
Lamb, L. E., Bartolone, S. N., Ward, E., and Chancellor, M. B. (2020). Rapid Detection of Novel Coronavirus (COVID-19) by Reverse Transcription-Loop-Mediated Isothermal Amplification. 1-17.
Mori, Y., and Notomi, T. (2009). Loop-mediated isothermal amplification (LAMP): a rapid, accurate, and cost-effective diagnostic method for infectious diseases. Journal of Infection and Chemotherapy 15, 62-69.
Nagamine, K., Hase, T., and Notomi, T. (2002). Accelerated reaction by loop-mediated isothermal amplification using loop primers. Molecular and Cellular Probes 16, 223-229.
Notomi, T., Okayama, H., Masubuchi, H., Yonekawa, T., Watanabe, K., Amino, N., and Hase, T. (2000). Loop-mediated isothermal amplification of DNA. Nucl. Acids Res. 28, E63-E63.
Yang, W., Dang, X., Wang, Q., Xu, M., Zhao, Q., Zhou, Y., Zhao, H., Wang, L., Xu, Y., Wang, J., et al. (2020). Rapid Detection of SARS-CoV-2 Using Reverse transcription RT-LAMP method. 1-25.
Zhang, Y., Odiwuor, N., Xiong, J., Sun, L., Nyaruaba, R. O., Wei, H., and Tanner, N. A. (2020). Rapid Molecular Detection of SARS-CoV-2 (COVID-19) Virus RNA Using Colorimetric LAMP. 1-14.

### Example 2—Protocols for Population-Scale Testing for SARS-CoV-2

### Protocol B.

1. A fresh swab sample is inserted into a 500 μl RT-LAMP reaction, containing the following components:
a. 250 μl WarmStart LAMP 2× Master Mix (NEB),
b.

1.6 uM total total of a set of 1-10 (preferentially 3) barcoded FIP primers
(TCTGGCCCAGTTCCTAGGTAGTNNNNNNNNNNCCAGACGAATTCGTGGTGG
(SEQ ID NO: 163)),

where Ns denote a specific barcode sequence,
c.

1.6 μM BIP primer
(AGACGGCATCATATGGGTTGCACGGGTGCCAATGTGATCT
(SEQ ID NO: 164)),

d.

0.2 μM F3 primer
(TGGCTACTACCGAAGAGCT (SEQ ID NO: 165)),

e.

0.2 μM B3 primer
(TGCAGCATTGTTAGCAGGAT (SEQ ID NO: 166)),

f.

0.4 μM LF primer
(GGACTGAGATCTTTCATTTTACCGT (SEQ ID NO: 167)),

g.

0.4 μM LB primer
(ACTGAGGGAGCCTTGAATACA (SEQ ID NO: 168)),

h. 5 μl Triton X-100,
i. a dilute control template DNA or RNA differing from the target viral sequence, but sharing primer binding sites,
j. water.

2. The RT-LAMP reaction with the swab is heated to 65 C for 30 minutes to react, and to 95 C for 10 minutes to sterilize.

3. 100,000 reactions are pooled, and a 10-cycle 100 μl PCR reaction is performed:
a. 50 μl NEBNext 2× Master Mix (NEB),
b.

0.5 μM B_fwd_10 primer
(ACACTCTTTCCCTACACGACGCTCTTCCGATCTCCGTCTTTGTTAGCA
CCATAGGG (SEQ ID NO: 169)),

c.

0.5 μM B_rev_12
(TGACTGGAGTTCAGACGTGTGCTCTTCCGATCTACCATCTTGGACTGA
GATCTTTC (SEQ ID NO: 170)),

d. 5 μl of pooled RT-LAMP products,
e. water.

4. A secondary 10-cycle 100 μl PCR reaction is performed with:
a. 50 μl NEBNext 2× Master Mix (NEB),
b.

0.5 μM D501 primer
(AATGATACGGCGACCACCGAGATCTACACTATAGCCTACACTCTTTCC
CTACACGACGCT (SEQ ID NO: 171)),

c.

0.5 μM D701 primer
(CAAGCAGAAGACGGCATACGAGATCGAGTAATGTGACTGGAGTTCAGA
CGTGTGCT (SEQ ID NO: 172)),

d. 5 μl of previous PCR reaction,
e. water.

5. The PCR product band is purified and sequenced on an Illumina NextSeq sequencer, or similar device.

6. Computationally, the barcodes co-occurring with the correct viral genome sequence (excluding the sequences covered by primers) are determined.

7. Positive patient samples are determined by their unique combination of barcodes using a decompression algorithm.

### Protocol C.

1. The patient receives a kit, containing:
a. Swab;
b. Tube containing: water, buffer, salts, lysis reagent, RT enzyme, DNA polymerase, RT-LAMP primers one barcoded, and optionally a dilute control template. The tube has a printed barcode on it;
c. A franked shipment envelope.

The barcoded primers in each tube are 6 RT-LAMP primers (examples published for SARS-COV-2 (Yang et al. “Rapid Detection of SARS-CoV-2 Using Reverse transcription RT-LAMP method” medRxiv (2020); Lamb et al. “Rapid Detection of Novel Coronavirus (COVID-19) by Reverse Transcription-Loop-Mediated Isothermal Amplification” medRxiv 2020)) with an inserted barcode in one of them. The barcode is linked by a database to the barcode printed on the patient's shipment envelope.

2. The patient takes a nasal swab and places it in the supplied tube. In some embodiments, the barcode could be a 10-nucleotide sequence inserted into the FIP (F1c+F2) or BIP (B1c+B2) primer, which thereby is inserted into the loop region of the LAMP product.

3. The patient closes the tube and places it in the shipment envelope and writes his name on it. While the oven heats up, it will pass a temperature range enabling viral lysis and exponential amplification of a snippet of viral genome sequence using an RT-LAMP reaction (50-72° C.). The time may not suffice for direct detection of DNA, but should suffice for downstream PCR amplification.

4. The patient places the whole envelope in the kitchen oven, and sets it to 100° C. Enzymes in the tube are heat-stabilized reverse transcriptase, heat-stabilized Bst polymerase, and/or variants thereof.

5. The patient ships the package to a central facility. Including a dilute control template in the tube with a sequence partially differing from the virus allows to monitor successful heat processing by the patient.

6. The central facility opens the tubes, scans their barcodes, and mixes them (up to 1 million samples per run). Optionally, the supplied tube might contain an insulation layer or other technical solution to slow down the temperature ramp in the contained solution.

7. A PCR is performed on mixed samples, adding Illumina-compatible handles. As the oven heats beyond reaction temperature, it inactivates the enzymes and sterilizes remaining virus, allowing non-hazardous shipment.

8. The PCR product is purified and sequenced on a NextSeq or similar sequencer. The samples mixed at the facility are computationally selected to not contain overlapping barcode sequences. Eventually, millions of barcodes could be synthesized and processed in parallel, but a set of 10k would suffice for scalable testing.

9. Computationally, person-specific barcodes occurring with correct viral sequence are identified. The PCR before deep sequencing faces the challenge that the LAMP products are long palindromes of viral sequence. Potential solutions I would like to test:
a. The mixed template is purified and tagmented by Tn5 with two handles before PCR
b. Two PCR primers with different Illumina handles are used which share the same binding sequence to the viral sequence.

### Note 1. Proposed Protocol and Logistics for Deployment Scenario One.

All steps below are performed by one sequencing center or one testing center per one day. A total of ten sequencing centers and 1,000 testing centers are predicted to operate in the whole country. According to below protocols, each sequencing and testing center would be staffed by two operators.

### Step 1: Preparing Reagent Plates (at Sequencing Center)

1. Using a MultiDrop reagent dispenser, 495 μl of RT-LAMP mastermix lacking template and FIP primer are dispensed into 960 96-well deep-well plates (“reagent plates”). Plates are stored in a cold room.
2. Using a Beckmann Biomek robot, for each of 10 barcode source plates (A-J):
   1. An operator places the source plate on the robot deck and unseals it,
   2. An operator places 96 reagent plates in the robot's hotel (alternative: process plates in smaller batches),
   3. An operator places 8 96-well tip boxes in the robot's hotel,
   4. In sets of 12 reagent plates (to be performed 8 times per source plate):
      a. The robot retrieves 12 reagent plates from the hotel and places them on the deck,
      b. Using the 96-well head, the robot aspirates 65 μl of primer solution per well from the source plate,
      c. Without changing tips, the robot dispenses 5 μl of primer solution into each of 12 reagent plates present on the deck,
      d. The robot discards the tips,
      e. The robot returns the 12 reagent plates to the hotel.
   5. The operator removes the 96 reagent plates from the hotel, seals them, and stores them for shipping in a cold room.

3. A courier delivers 10 reagent plates with orthogonal barcodes A-J to each testing center, located within a distance of 300 km from the sequencing center. A total of 96 testing centers are served by each sequencing center.

### Step 2: Processing Swab Samples (at Testing Center)

1. For each of 10 reagent plates:
   a. The plate is unsealed and placed in a sterile hood, preferentially on a cooling device,
   b. Swab samples are taken by a medical worker using personal protective equipment from individuals at a rate of roughly two samples/minute,
   c. Swabs are inserted in successive order into the wells of the reagent plate,
   d. The last two wells of every plate are left empty/spiked in with a positive control RNA,
   e. When the plate is full, it is sealed using an adhesive foil, and incubated in the sterile hood in two ovens (20 minutes 65° C., 10 minutes 95° C.),
   f. The plate is stored at room temperature.
2. At the end of the day or when all plates have been processed, an operator unseals all plates in a sterile hood, and pours them into a plastic container.
3. The combined liquid in the container is mixed, and a small sample is transferred to a 2 ml plastic screw cap tube.
4. A courier delivers the tube to the serving sequencing center.

### Step 3: Processing and Sequencing Pooled Samples (at Sequencing Center)

1. An operator gathers 96 pool tubes from testing centers in a rack, and dispenses 100 μl of each pool into a 96-well plate with well positions according to the testing center ID.
2. The operator prepares a PCR plate with 95 μl mastermix per well (water, 2× NEBNext mastermix, two primers).
3. The operator stamps over 5 μl of each template into the PCR plate using a 12-channel pipet.
4. The operator runs the PCR plate in a 96-well temperature cycler according to the protocol.
5. The operator unseals the plate.
6. The operator prepares a secondary PCR plate with 95 μl mastermix per well (water, 2× NEBNext mastermix, no primers).
7. The operator stamps in 95 unique primer combinations from a stock plate using a 12-channel pipet.
8. The operator stamps over 5 μl from the first PCR into the secondary PCR plate using a 12-channel pipet.
9. The operator runs the PCR plate in a 96-well temperature cycler according to the protocol.
10. On ice, the operator pools 20 μl of each of 96 secondary PCR reactions in a reservoir.
11. The operator runs the pool on a 2% EX E-Gel (10 pockets×10 μl pool per pocket).
12. The operator cuts the bands of appropriate size from the gel, and purifies the DNA using a Qiagen agarose gel purification kit according to the manufacturer's instructions.
13. The operator re-purifies the DNA using a Qiagen PCR purification kit according to the manufacturer's instructions.
14. The operator quantifies the DNA using a NanoDrop photospectrometer or alternative device.
15. The operator thaws a NextSeq High-Output 75-cycle kit (Illumina), inserts the DNA, and initiates a run, which takes 14 hours.

### Step 4: Data Analysis

1. An operator transfers the NextSeq data to a server computer.
2. The operator initiates barcode deconvolution using a predefined SampleSheet and the bcl2fastq tool (Illumina), which takes 2 hours.
3. The operator inflates the compressed output files on the server, resulting in FASTQ files of about 50 GB in total size, which takes 1 hour.
4. The operator executes a software program that takes in 96 FASTQ files, and saves a list of positive/negative/unresolved sample barcodes, which takes 4 hours.
5. The operator copies the list to an online server, and executes a software program that re-formats the data and adds them to a database, suited for anonymous web-based retrieval of testing results based on user ID.

### Example 3—SARS-CoV-2 Detection as a Bloom Filter

Provided herein is a method to scalably detect SARS-CoV-2-infected patients. The goal is to model various approaches for doing so, and characterize the error rates of each approach. This Example assumes that there are two stages of barcoding available: patient samples will be individually barcoded at the RT-LAMP stage with a first set of barcodes (barcode 1), and then groups of patient samples will be further barcoded in the process of preparing samples for Illumina sequencing with a second set of orthogonal barcodes (barcode 2). After sequencing, a given barcode pair can be called as positive or negative for SARS-CoV-2 viral RNA. Let there be m total unique barcode 1s and m₂ total unique barcode 2s.

Suppose that one wants to test N total patient samples, which will be done in batches of size b at once with k pre-assigned barcode 1s per patient sample. Further suppose that a fraction p of the total population is positive for SARS-CoV-2. Then, ≈n=pb samples in each batch will be positive. There are two types of general approaches considered below, corresponding to the two deployment scenarios in the main text—one where k=1 and one where k>1.

To compute error rates from any approach, one must model for how errors. It is assumed that errors from barcode loss and sample skewing dominate, modeling barcode loss first and then sample skewing.

### 1. Modeling Barcode Loss.

To compute error rates for any approach, one must first have a model for barcode loss. It is assumed that barcode loss occurs only for barcode 1s, and that there are two types of errors. This is because there are many copies of a sample before amplification and addition of barcode 2, and because barcode 2 failure is easy to detect. One will also assume that no negative barcodes will show up as positive. This is reasonable as template switching is unlikely in RT-LAMP reactions.

The first type is an error where a given barcode never functions properly, which will happen with some probability Δ_{synth}. This may be because it never got synthesized properly, or because it somehow impedes amplification. This probability has not been characterized, but if the barcodes are designed with sufficient edit distance, one-bit error correction can be implemented, so it can be expected to be low. Overall, after filtering, it is expected this to be low (about 0.01).

The second type is an error where a given barcode may not be picked up for a particular positive sample with probability Δ_{stoch}. This may be due to dilution effects or sample skewing errors. This probability will likely vary with the number of people infected in a batch n, so it should also vary with the proportion of the population infected p and the batch size b. This probability has also not been fully characterized.

2. Scenario 1: k=1.

If b<m·m₂, then every sample in batch can be assigned a unique barcode. It is challenging to synthesize enough barcodes for N<m·m₂, which would allow a unique barcode for every sample from the total population, so batches would need to be defined in some way. This suggests this scenario would likely need asynchronous testing. Every patient sample in a batch would then have a unique barcode 1-barcode 2 pair. In other words, one setup for doing so would be to give up to m different patient samples at a testing site each one of the m barcodes, so that none overlap. Then, all of the samples at a testing site would be pooled together and assigned a single barcode 2 that differs from all other test sites. At the final sequencing center, all barcoded samples would be pooled and run together as one batch. Every patient sample in a batch would then have a unique barcode 1-barcode 2 pair that differs from every other sample.

Since each patient has a unique barcode pair, there are no false positives. However, with barcode loss, there may be false negatives. Each patient sample would have a false negative probability of Δ_{stoch}+Δ_{synth} (1−Δ_{synth}).

3. Scenario 2: k>1

With a liquid handler like an Echo, it may be possible to give each patient sample k different barcode 1s, with k>1, in a pre-assigned way. Then, if N<(m/k), every patient sample for the entire population could have a unique combination of barcode 1s. This does not mean that every barcode 1 would correspond to a unique patient sample, as this is only possible if N<m/k.

For this scenario, imagine an asynchronous sample collection system where b of these samples, as they come in, are split into m₂ sub-batches. Then, each sub-batch get a unique barcode 2. If, after sequencing, ≥k′ out of the k barcode pairs that correspond to a specific patient sample come up as positive, one would say that that patient sample is positive.

Since the barcode 1s no longer corresponds to unique patient samples, there is a potential for false positives using this approach. False negatives are also still possible, as a positive sample could be called as negative if more than k′ of the k corresponding barcode pairs are lost. Using a modified Bloom filter model (see “Modeling as a Modified Bloom Filter”), one can compute the false positive probability (FPP_{Δk′,m2}) and false negative probability (FNP_{Δk′,m2}) for this approach as (Eq. 1)

$${FPP}_{Δ {k}^{′} , {m}_{2}} = {\sum }_{i = {k}^{′}}^{k} ( \begin{matrix}k \\ i\end{matrix} ) {( {Δ}_{synth} + ( 1 - {Δ}_{synth} ) ( 1 - \frac{1 - {Δ}_{stoch}}{m} ) \text{?} )}^{k - i} {( ( 1 - {Δ}_{synth} ) ( 1 - ( 1 - \frac{1 - {Δ}_{stoch}}{m} ) \text{?} ) )}^{i} \qquad ( 1 )$$

$${FNP}_{Δ {k}^{′} , {m}_{2}} = 1 - {\sum }_{i = {k}^{′}}^{k} ( \begin{matrix}k \\ i\end{matrix} ) {( {Δ}_{synth} + ( 1 - {Δ}_{synth} ) {Δ}_{stoch} ( 1 - \frac{1 - {Δ}_{stoch}}{m} ) \text{?} )}^{k - i} {( ( 1 - {Δ}_{synth} ) ( 1 - {Δ}_{stoch} ( 1 - \frac{1 - {Δ}_{stoch}}{m} ) \text{?} ) )}^{i} .$$

$$\text{?} \text{indicates text missing or illegible when filed}$$

Note that these error rates are both dependent on the proportion of the population infected, p, since n=pb. As p increases, the false negative probability will drop, as other positive patient samples can compensate for the barcode loss of a given sample. One can compute an upper bound on the false negative probability (FNP_{Δk′,m2, max}) that avoids this p dependence as (Eq. 2)

$${FNP}_{Δ {k}^{′} , {m}_{2} , max} = 1 - {\sum }_{i = {k}^{′}}^{k} ( \begin{matrix}k \\ i\end{matrix} ) {( {Δ}_{synth} + ( 1 - {Δ}_{synth} ) {Δ}_{stoch} )}^{k - i} {( ( 1 - {Δ}_{synth} ) ( 1 - {Δ}_{stoch} ) )}^{i} . \qquad ( 2 )$$

Additionally, the barcode loss lowers the false positive probability in this scenario. Since Δ_{stoch} and Δ_{synth} are both uncharacterized, it may be worth considering the case where there is no barcode loss (Δ_{stoch}=Δ_{synth}=0) for robustness. Then, the false positive probability, (FPP_{Δk ′,m2, max}), which is still a function of p, can be given as (Eq. 3)

$${FPP}_{Δ {k}^{′} , {m}_{2} , max} = {\sum }_{i = {k}^{′}}^{k} ( \begin{matrix}k \\ i\end{matrix} ) {( ( 1 - \frac{1}{m} ) \text{?} )}^{k - i} {( 1 - ( 1 - \frac{1}{m} ) \text{?} )}^{i} . \qquad ( 3 )$$

$$\text{?} \text{indicates text missing or illegible when filed}$$

Overall, a lower k′ for a fixed k leads to a reduced false negative probability at the cost of an increased false positive probability. The effect of varying k depends on the chosen k′ and on the proportion of the population infected p.

### Scenario Comparison.

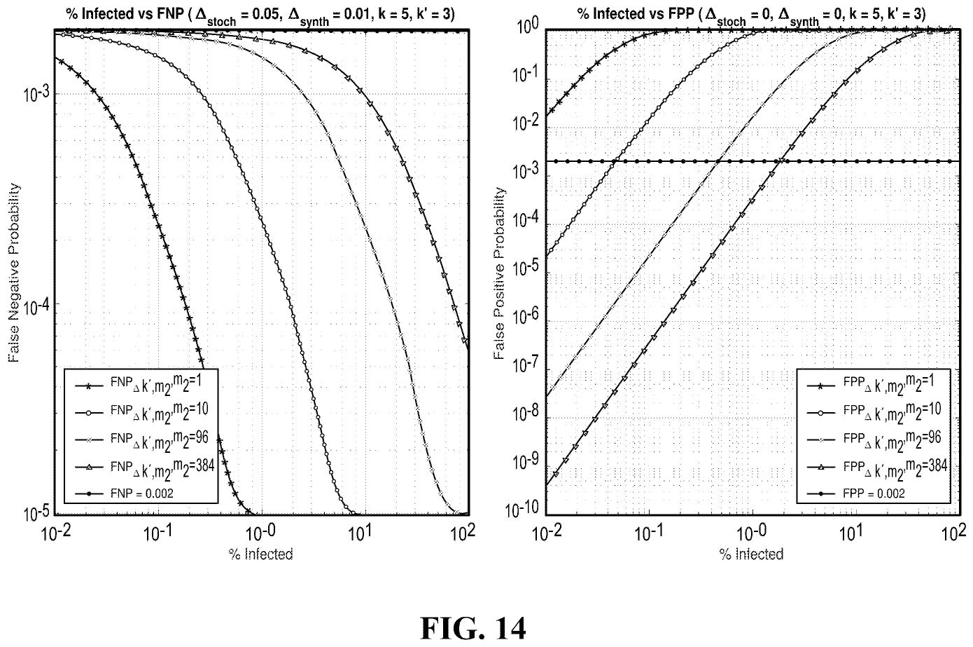

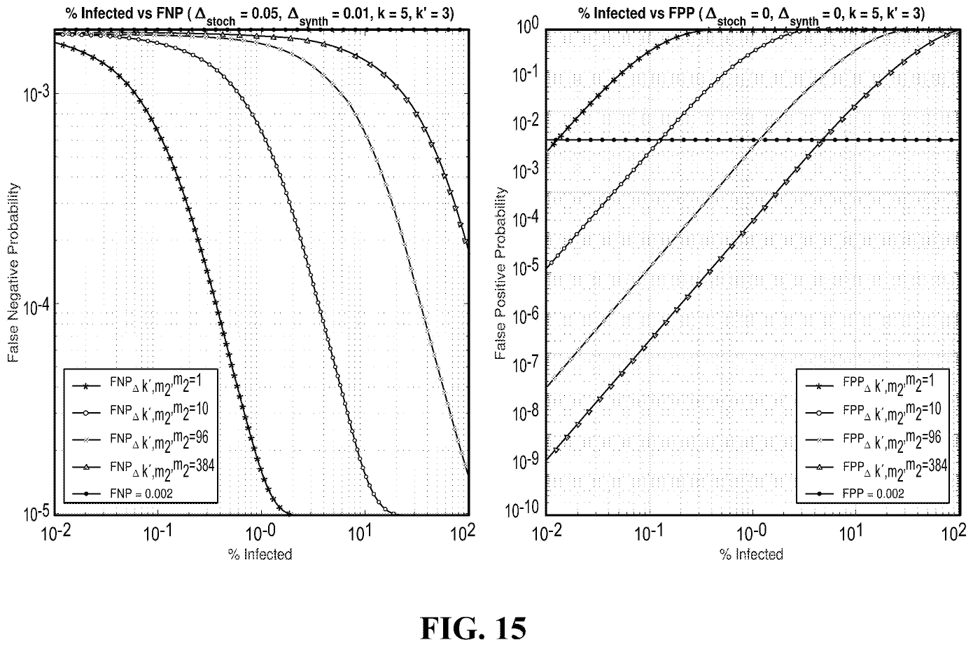

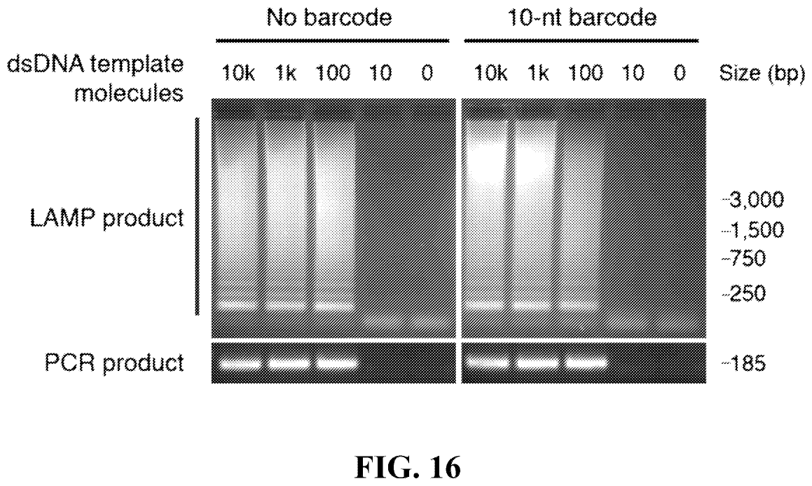

With a carefully chosen parameter set, scenario 2 can produce a lower false negative probability relative to scenario 1 at the cost of an increased false positive probability. Comparison of these two scenarios using realistic numbers can be found below. Some additional error plots with other values for m are shown in FIGS. 14 and 15.

### 4. Modeling Sample Skewing

Sample skewing errors could also occur. Patient viral loads from nasopharyngeal swabs vary over many orders of magnitude across the course of infection (2). This variation could lead to over-representation of some positive samples, preventing detection of samples with lower viral abundance and giving rise to false negatives.

RT-LAMP and PCR are both nonlinear amplification methods, and how patient viral load variation propagates through both techniques remains to be experimentally determined. To initially model this using numerical simulation, we consider two possibilities.

One possibility is that the saturation of both RT-LAMP and PCR lead the distribution of barcoded molecules post-PCR to have less variation than the original viral load. This was modeled by drawing the molecules post-PCR for a barcode from a normal distribution with mean 10⁴ and standard deviation 10³, with the same number of molecules for each of the k barcodes for a positive sample. This is referred to as “Saturated”. Initial data collection suggests that LAMP-seq leads to saturation, so we assume this is the more accurate model.

Another possibility is that the RT-LAMP and PCR lead to the retention or exacerbation of the initial sample variation. This was modeled by drawing the molecules post-PCR for a barcode from a log-normal distribution with mean 4:5 and standard deviation 3, with the same number of molecules for each of the k barcodes for a positive sample. This is referred to as “Amplified”.

Overall, the modeled molecules per sample is not important, and may be orders of magnitude higher, as the relative abundance of various barcodes is what will determine false negatives.

Assuming that an Illumina NextSeq run generates 200 million total sequencing reads and that about 90% of the reads are usable, there are a total of. About 18 million reads per sub-batch. In the simulation, a given barcode was called as positive if the number of reads for the barcode (calculated by relative abundance multiplied by reads per sub-batch) was greater than or equal to a threshold of t reads. Otherwise, it was called as a negative barcode.

Using the parameters for Scenario 1 and 2 (k1=3 and k=5) without barcode loss, we calculated the average FPPs and FNPs over 1000 iterations for various threshold values t, shown in Table 4. Under the “Saturated” model, sample skewing introduces minimal false negatives. With a threshold of 100 reads, the FNP is tolerable for both scenarios. For the “Amplified” model, sample skewing introduces more false negatives, with the threshold reads t determining exactly how many. As the proportion of the population infected p goes up, the FNP will increase.

TABLE 4
FPP/FNP for various t with b = 10⁵, m =
10⁴, m₂ = 10, and p = 0.01.

| Threshold | | | | |
|---|---|---|---|---|
| Reads t | Average FNP | Average FPP | Model | Scenario |
| 100 | 0.0000 | 0.0000 | Saturated | 1 |
| 2 | 0.0087 | 0.0000 | Amplified | 1 |
| 10 | 0.0296 | 0.0000 | Amplified | 1 |
| 100 | 0.1227 | 0.0000 | Amplified | 1 |
| 100 | 0.0000 | 0.0010 | Saturated | 2 |
| 2 | 0.0289 | 0.0010 | Amplified | 2 |
| 10 | 0.0817 | 0.0009 | Amplified | 2 |
| 100 | 0.2567 | 0.0005 | Amplified | 2 |

It was assumed here that either sample skewing or barcode loss dominates. Combining both may produce a more realistic model. Simulations doing so are available in the Github repository.

### 5. Other Potential Approaches.

The two scenarios presented here are not the only possible approaches. There are other approaches that may have lower error rates. Some scenarios where either the barcode is or barcode 2s are not utilized are modeled in “Modeling as a Modified Bloom Filter” below, and some more complex scenarios with redundancy across barcode 2s are briefly explored in “More Complex Scenarios”.

### Modeling as a Modified Bloom Filter.

A useful first approximation for this problem is that of a Bloom filter. Using an idealized Bloom filter some theoretical limits and optima if one only uses barcode 1s were calculated. The Bloom filter is then modified to incorporate barcode loss, as well as different criteria for calling a sample as positive, before adding in the barcode 2s to produce a final model. All derivations can be found in the Math Appendix (Section 8) of this Example.

The methods provided herein allow for scalable detection of SARS-CoV-2-infected patients given a limited number m of total unique barcodes available. Query for whether a barcode in a test batch is positive or negative for SARS-CoV-2 is done by sequencing. This query is assumed for now to be deterministic and always succeed, to be relaxed later. Suppose that one wants to test N total patient samples. If N≤m, each sample could be assigned a unique barcode. However, realistically N>m, so samples are tested in batches of size b at once with k pre-assigned barcodes per patient sample. Further suppose that a fraction p of the total population is positive for COVID-19. This means that one expects n=pb samples in each batch to be positive. Then, this problem can be modeled as a Bloom filter, calculating theoretical limits. Assumptions for this approximation are in section 3 below, followed by a model which includes barcode errors. Various strategies are then explored for lowering error rates, with a goal of producing a realistic model for a real diagnostic test for population-scale testing.

### 1.1 Bloom Filters and Correspondence to the Problem.

A general Bloom filter is a data structure that can be used to test whether an element is a part of a set of cardinality n (Burton et al. 1970. Commun. ACM 13(7_:422-426). This test returns either “definitely not”, or “possibly yes”. A Bloom filter is implemented by taking a bit array of size m, initialized at 0. To add an element to the filter/set, k hash functions are used to map the new element to k bits in the array. These k bits are then set to 1, if they are not already 1. To query whether a given element is in the set, compute the k hashes again. If any of the corresponding k bits in the array are 0, then the element is not in the set. If they are all 1, then the element may be in the set.

It is assumed here that there are no barcode 2s for now (i.e. m₂=1). Then, a Bloom filter can be generated for every batch of size b tested. Here, elements are individual samples, and inclusion in the set of cardinality n=pb corresponds to being SARS-CoV-2 positive. The m barcodes correspond to the m bits in the bit array, where being set to 0/1 means it was detected as SARS-CoV-2 negative or SARS-CoV-2 positive respectively. Similarly, the outputs of the k hash functions are equivalent to the k barcodes assigned to a given sample.

### 1.2 Underlying Assumptions

The critical assumption of this model that is broken in real life is the idea that a barcode will always turn up positive if it is positive, implying no barcode loss. This is addressed and modeled in later sections, but it still useful to consider this initial idealized case for now.

Otherwise, the other assumptions mostly hold. Namely, the k hash functions are supposed to be independent and produce a random uniform distribution across the keys (different barcodes). This can be achieved using a proper barcode assignment. Another assumption is that barcodes will not spontaneously turn positive, which should be the case under this barcoding scheme. Bits are assumed to be set to either 0 or 1, which is realistic if we binary threshold the reads for a given barcode.

### 2. Single Bloom Filter, No Barcode Loss.

### 2.1. False Positives/Negatives.

If an element is in the set, all k bits will be set to 1, so false negatives are not possible. However, if, by chance, all k corresponding bits in the filter for an element e are set to 1 due to other elements that combined to map to the same k bits, one will mistakenly claim that e is in the set, even if it is not. So false positives are possible, analogous to hash collisions. This false positive rate is (Eq. 4)

$$Pr [ False Positive ] = {( 1 - {( 1 - \frac{1}{m} )}^{kn} )}^{k} \approx {( 1 - {e}^{- \frac{kn}{m}} )}^{k} . \qquad ( 4 )$$

This makes certain assumptions about the hash functions, but these are satisfied by the barcode assignment (see the Math appendix (Section 8) of this Example).

### 2.2. Optimal Choice of k.

There is an optimal choice for k to minimize the false positive rate. This value is very closely approximated by kOPT=ln(2)m/n with a corresponding false positive probability (FPP) of 0.5^{kOPT}, derived below. Note that what matters is the ratio m to n, so things can scale accordingly with various numbers of barcodes as long as the batch size to barcode number ratio remains the same for a given % infected.

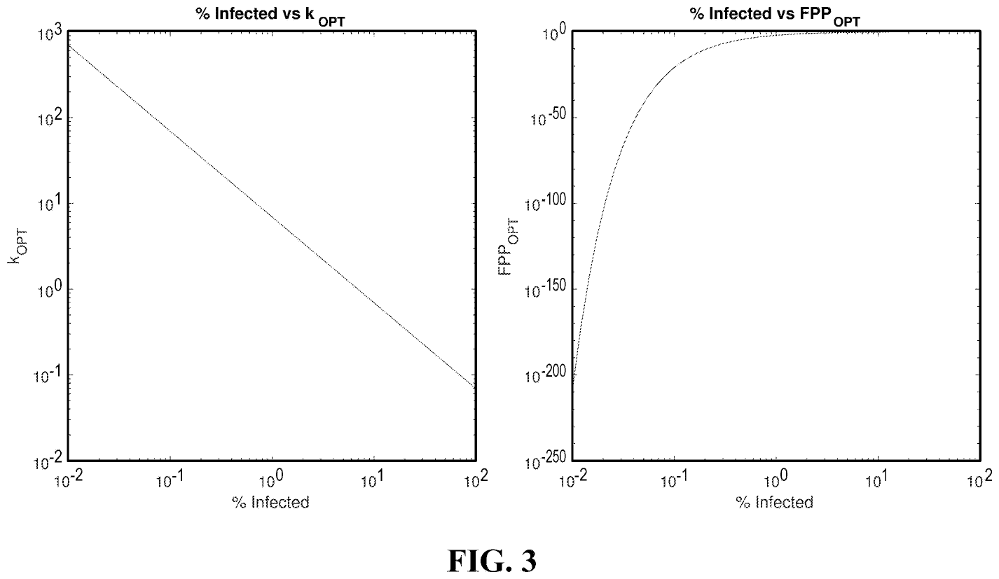

If one chooses m=10,000 barcodes and test batches of size b=100,000 at once, then one can plot the optimal choice of k given the percent of the population infected, as well as the corresponding false positive probability (FIG. 3). One can also generate a table showing the optimal k given a certain percentage of the population infected (Table 5).

When the percentage infected is low, kOP T is high, and leads to a low FPP. As the percentage infected rises, kOP T will drop, due to barcode saturation, and the FPP rises. So, in early stages, high values for k are optimal, and this shrinks across the course of the pandemic. However, adjusting k as the pandemic evolves may not be realistic. High k may also cause technical problems with the RT-LAMP reaction. Instead, the focus is on what happens if we pre-choose k=2, 3, 4 or 5.

TABLE 5
k_{OPT} and False Positive Probability
(FPP) assuming m = 10⁴ barcodes

| | | % Infected | % Infected | |
|---|---|---|---|---|
| k_{OPT} | n(Infected/Batch) | (b = 10⁴) | (b = 10⁵) | FPP |
| 138.63 | 50 | 0.5 | 0.05 | 1.85 × 10⁻⁴² |
| 69.3 | 100 | 1 | 0.1 | 1.37 × 10⁻²¹ |
| 34.66 | 200 | 2 | 0.2 | 3.68 × 10⁻¹¹ |
| 13.86 | 500 | 5 | 0.5 | 6.73 × 10⁻⁵ |
| 6.93 | 1000 | 10 | 1 | 0.00820 |
| 5 | 1386 | 13.86 | 1.386 | 0.03125 |
| 4 | 1733 | 17.33 | 1.733 | 0.0625 |
| 3 | 2311 | 23.11 | 2.311 | 0.125 |
| 2 | 3466 | 34.66 | 3.466 | 0.250 |
| 1.38 | 5000 | 50.00 | 5 | 0.384 |

As an example, Germany has a total population of 82.79 million and there were ≈44,000 positive cases in Germany as of 3/27, so the current percentage infected is ≈0.053%. So, k_{OPT} is currently very high with a very low FPP, and as the percentage infected rises, k_{OPT} will drop and FPP will rise for Germany. However, adjusting k as the pandemic evolves may not be realistic. High k may also cause technical problems with the RT-LAMP reaction. Instead, focus will be on what happens if one pre-chooses k=2, 3, 4 or 5.

2.3. k=2, 3, 4\hat{ }5.

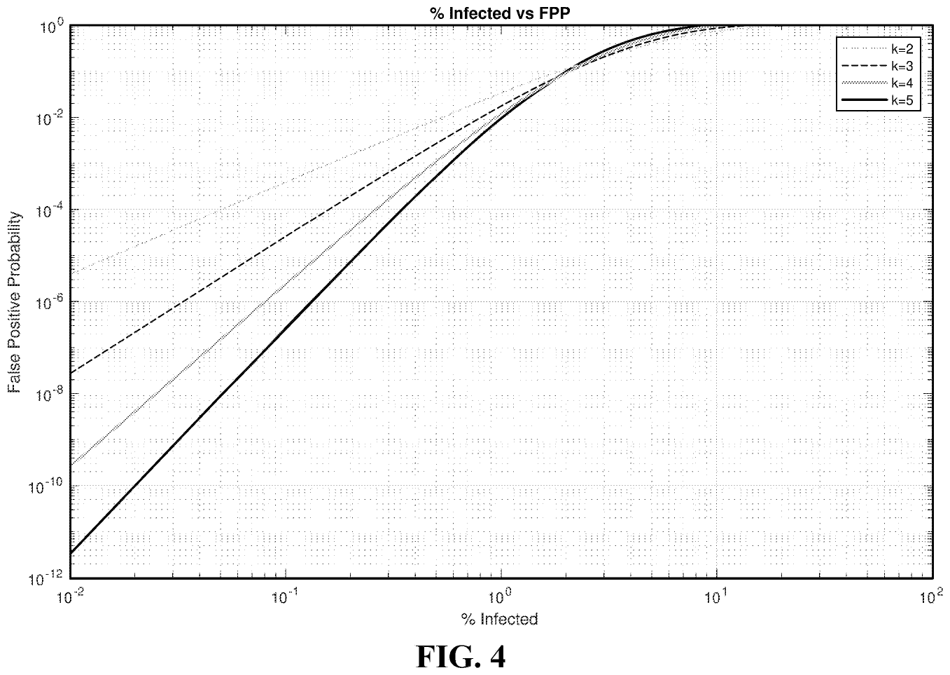

When operating with k=2, 3, 4 or 5, one is not very close to the current theoretical optimum. However, it may be the best that can be technically done. Note that the FPP can still be quite low, even with such a low k (FIG. 4).

As an alternative approach, given an error threshold E on the FPP, one can solve for a maximum compression factor c=b/m, derived below. This constraint is (Eq. 5)

$$e = \frac{- ln ( 1 - {ϵ}^{\frac{1}{k}} )}{kp} . \qquad ( 5 )$$

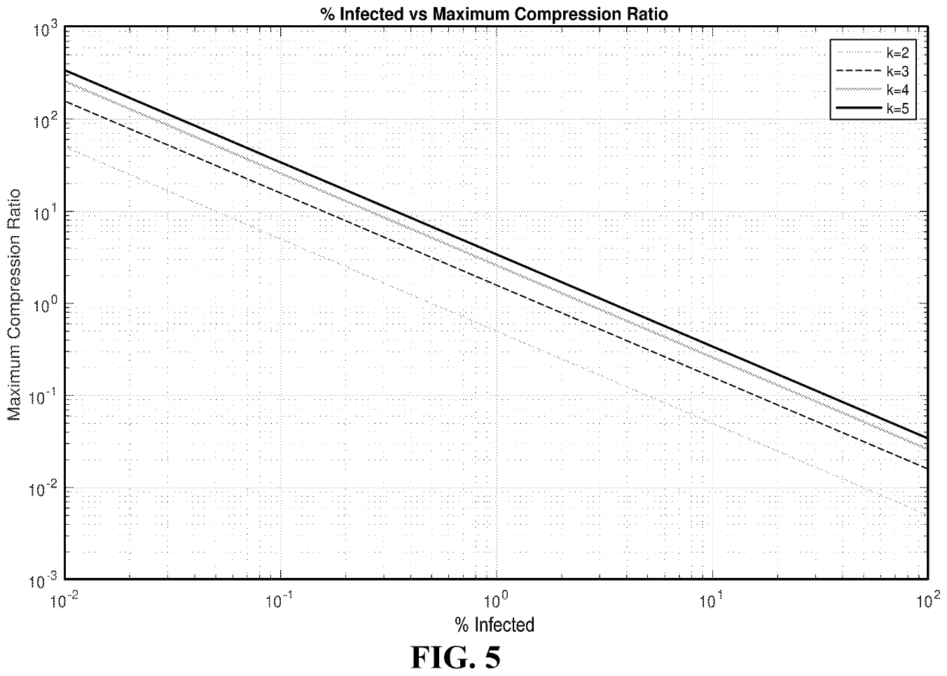

This c can be interpreted as the maximum number of samples per barcode that can be run given an error threshold c, k barcodes per sample, and some proportion p infected. If one chooses ε=0.0001 or 0.01%. Then, one can examine the maximum theoretical compression factor for various k as the % infected varies (FIG. 5).

One can also calculate the max % infected that ensures that the error rate is still under threshold (Table 6). Note that scaling down the number of samples per batch by a factor s also scales up the maximum percentage infected by a factor s, as the maximum % infected is inversely proportional to b for a chosen error rate. For this regime, increasing k will continue to raise the maximum % infected, before coming crashing down.

TABLE 6
Max % infected for various k and FPPs

| | | % Infected Max | % Infected Max |
|---|---|---|---|
| FPP (in %) | k | (b = 10⁴) | (b = 10⁵) |
| 0.01 | 2 | 0.503 | 0.050 |
| 0.02 | 2 | 0.712 | 0.071 |
| 0.05 | 2 | 1.131 | 0.113 |
| 0.1 | 2 | 1.607 | 0.161 |
| 0.01 | 3 | 1.584 | 0.158 |
| 0.02 | 3 | 2.009 | 0.201 |
| 0.05 | 3 | 2.757 | 0.276 |
| 0.1 | 3 | 3.512 | 0.351 |
| 0.01 | 4 | 2.634 | 0.263 |
| 0.02 | 4 | 3.165 | 0.317 |
| 0.05 | 4 | 4.049 | 0.405 |
| 0.1 | 4 | 4.895 | 0.490 |
| 0.01 | 5 | 3.451 | 0.345 |
| 0.02 | 5 | 4.019 | 0.402 |
| 0.05 | 5 | 4.935 | 0.494 |
| 0.1 | 5 | 5.785 | 0.579 |

The takeaway here is that for k=4, one can maintain a false positive probability of <0.01% with b=10⁵ and m=10⁴ if the total % infected remains under 0.263%, 5× the current percentage. If this is exceeded, the number of samples per batch can just be scaled down. If one reduces down to b=5×10⁴, % infected needs to remain below 0.526% to maintain this error rate. Similarly, 1.317% max for b=2×10⁴ and 2.634% max for b=10⁴. For k=5, one can maintain a FPP<0.01% with b=10⁵ and m=10⁴ if the total % infected remains under 0.345%, 9× the current percentage.

### 3. Underlying Assumptions.

The main underlying assumption here that is broken in real life is the idea that a barcode will always turn up positive if it is positive. This will be addressed in the next section. There may be ways to augment this and still maintain reasonable false negative and false positive rates.

Otherwise, the other assumptions mostly hold. Namely, the k hash functions are supposed to be independent and produce a random uniform distribution across the keys (different barcodes). This can be achieved using a proper barcode assignment. Another assumption is that barcodes will not spontaneously turn positive, which should be the case if the noise is low and there is no weird template switching in the reaction mix. Bits are assumed to be set to either 0 or 1, which seems realistic if one binary thresholds the reads for a given barcode. It could be nice to bin up the barcode reads further/make use of the counts for a given barcode, but the nonlinearity of amplification probably makes this hard.

4. Single Bloom Filter with Barcode Errors.

### 4.1 Modeling Barcode Loss.

This section is drawn to modeling what happens if the query for whether a barcode is positive fails sometimes, considering two types of errors. It is continued to be assumed that no negative barcodes will show up as positive.

The first type is an error where a given barcode never functions properly, which will happen with some probability Δ_{synth}. This may be because it never got synthesized properly, or because it somehow impedes amplification. This probability has not been characterized, but an initial guess (hopefully an overestimate) is Δ_{synth}=0.01. In the Bloom filter model, for any specific bit in the bit array, this corresponds to having the bit remain at 0, regardless of the number of attempts at flipping it, with a probability of Δ_{synth}.

The second type is an error where a given positive barcode may not be picked up for a particular sample with probability Δ_{stoch}. This may be due to dilution effects or sample skewing errors. This probability will probably vary with batch size b, but an initial guess for all b (hopefully an overestimate) is Δ_{stoch}=0.05. In the Bloom filter model, for any specific bit in the bit array that works, this corresponds to having the bit not flip for a given attempt at flipping it with a probability of Δ_{stoch}.

4.2 Error Rates with Barcode Loss.

The criteria with which a sample is called “positive” can be changed. In this first model, a sample will only be called positive if all k barcodes for the sample turn up positive. If at least one of the k barcodes is negative, it will be called negative.

One can calculate the False Positive Probability for this model (FPP), derived below, as (Eq. 6)

$${FPP}_{Δ} = {( 1 - {Δ}_{synth} )}^{k} {( 1 - {( 1 - \frac{1 - {Δ}_{stoch}}{m} )}^{kn} )}^{k} \approx {( 1 - {Δ}_{synth} )}^{k} {( 1 - {e}^{- ( 1 - {Δ}_{stoch} ) \frac{kn}{m}} )}^{k} . \qquad ( 6 )$$

The curve is affected dramatically, even with Δ_{synth}=0.01 and Δ_{stoch}=0.05. Note that non-zero Δ_{stoch} and Δ_{synth} only serve to lower this FPP compared to the original FPP with no errors, so the original FPP is an upper bound on the true FPP incorporating errors. Since one doesn't actually know the values of Δ_{stoch} or Δ_{synth}, it may be wise to proceed using the FPP derived with no errors. This would help ensure that models are robust regardless of the true values of the error rates.

Barcode errors also introduce false negatives, as there is some probability that at least one of the k corresponding barcodes for a given sample will not show up as positive even if it is positive. The False Negative Probability (FNPA) for this model, derived below, is (Eq. 7)

$$\begin{matrix}{FNP}_{Δ} = 1 - {( 1 - {Δ}_{synth} )}^{k} {( 1 - {{Δ}_{stoch} ( 1 - \frac{1 - {Δ}_{stoch}}{m} )}^{k ( n - 1 )} )}^{k} \\ \approx 1 - {( 1 - {Δ}_{synth} )}^{k} {( 1 - {Δ}_{stoch} {e}^{- ( 1 - {Δ}_{stoch} ) \frac{k ( n - 1 )}{m}} )}^{k}\end{matrix} \qquad ( 7 )$$

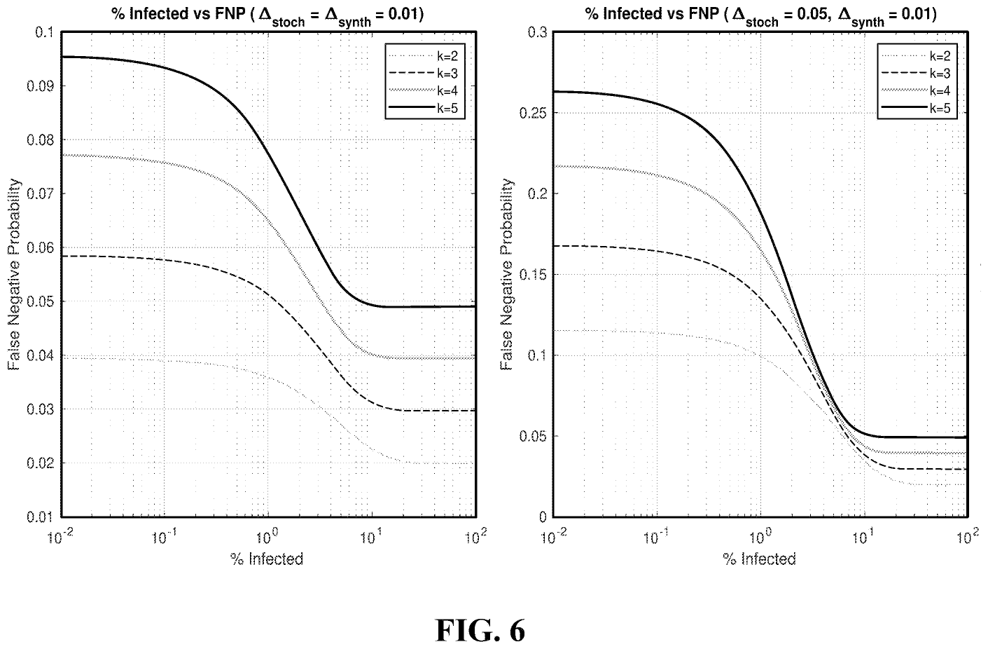

Since this is dependent on n, this probability will vary with the % of the population infected. The FNP_{Δ} curves for k=2, 3, 4 and 5 with various parameters are shown in FIG. 6.

For low % infected, a large component of the FNPΔ is contributed by Δ_{stoch}. As the % infected increases, this contribution drops, but may still dominate over Δ_{synth}. An upper-bound on this probability, FNP_{Δ, max}, is given by (Eq. 8)

FNP_{Δ,max}=1−(1−Δ_{synth})^{k}(1−Δ_{stoch})^{k}, (8)

For Δ_{synth}=0.01 and Δ_{stoch}=0.05, this is 0.12 for k=2, 0.17 for k=3, 0.22 for k=4, and 0.26 for k=5, all unacceptably high. If one can reduce both Δ_{synth} and Δ_{stoch} ten-fold, then FNP_{Δ, max} would become 0.012 for k=2, 0.018 for k=3, 0.024 for k=4, and 0.029 for k=5.

In this regime of k, as k increases, FPP_{Δ} will decrease and FNP_{Δ} will increase. So, there is a trade-off between false negatives and false positives as k is varied.

At the current values of % infected, FNP_{Δ} is much higher than FPP_{Δ}. For a good diagnostic, one wants to prioritize a low false negative probability, while still maintaining a low false positive probability. Next are a few different strategies for reducing the false negative probability. The first strategy will still be on a single batch tested once, and the remainder will involve a second barcode.

4.3 Calling Elements Positive if ≥k−1 Out of k Bits are Positive.

One potential strategy for augmenting a single Bloom filter with barcode errors is to change the criterion when querying whether a given element is in the set. This can be done by saying that an element is in the set if ≥k−1 out of the k corresponding bits are 1. This is equivalent to calling a sample positive if ≥k−1 out of its k corresponding barcodes are positive. This is generalizable to calling a sample positive if ≥k′ out of k barcodes are positive, but this was not done here because the k values are low and the relevant calculations are very bashy.

Under this scheme, one can calculate the error rates (FNP_{Δ2} and FPP_{Δ2}) as (Eq. 9)

$${FPP}_{Δ 2} = {FPP}_{Δ} + k ( {Δ}_{synth} + ( 1 - {Δ}_{synth} ) {( 1 - \frac{1 - {Δ}_{stoch}}{m} )}^{kn} ) {( ( 1 - {Δ}_{synth} ) ( 1 - {( 1 - \frac{1 - {Δ}_{stoch}}{m} )}^{kn} ) )}^{k - 1} \qquad ( 9 )$$

$${FNP}_{Δ 2} = {FNP}_{Δ} - k ( {Δ}_{synth} + ( 1 - {Δ}_{synth} ) {{Δ}_{stoch} ( 1 - \frac{1 - {Δ}_{stoch}}{m} )}^{k ( n - 1 )} ) {( ( 1 - {Δ}_{synth} ) ( 1 - {{Δ}_{stoch} ( 1 - \frac{1 - {Δ}_{stoch}}{m} )}^{k ( n - 1 )} ) )}^{k - 1} .$$

derived below along with exponential approximations. An upper bound on the false negative probability (FNP_{Δ2,max}) can be calculated as (Eq. 10)

FNP_{Δ2,max}=FNP_{Δ,max}−k(Δ_{synth}+(1−Δ_{synth})Δ_{stoch})((1−Δ_{synth})(1−Δ_{stock}))^{k-1}, (10)

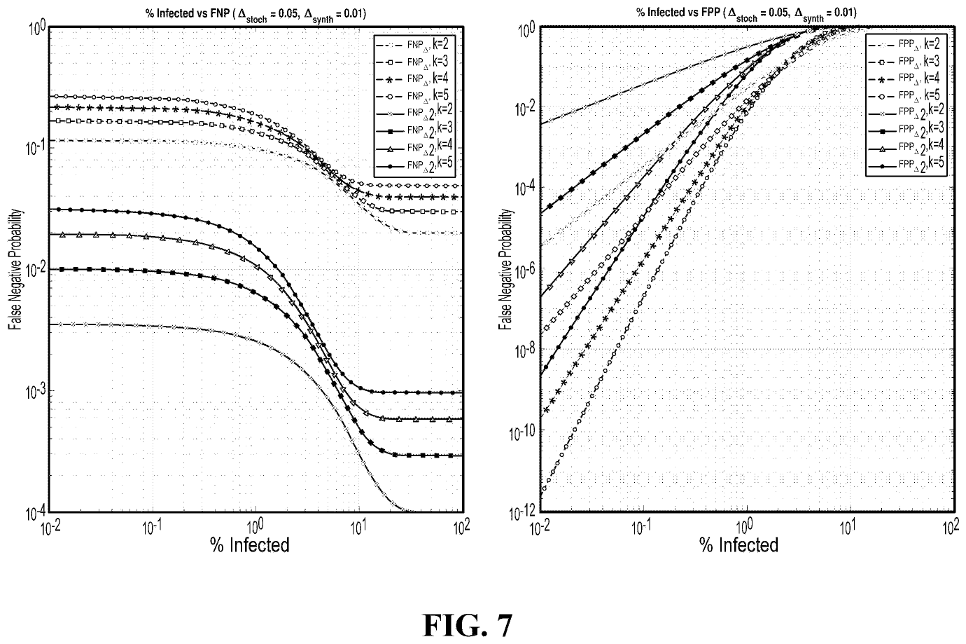

This upper bound on the false negative probability, for Δ_{synth}=0.01 and Δ_{stoch}=0.05, is 0.0035 for k=2, 0.010 for k=3, 0.020 for k=4, and 0.031 for k=5, much lower than before. A comparison between FNP_{Δ2} and FPP_{Δ2} and the original FNP_{Δ} and FPP_{Δ} is shown in FIG. 7.

This strategy allows for a much lower false negative probability, at the cost of a higher false positive probability. With k values that are so low, this might not be a reasonable approach, but it sets the stage for more complex strategies that can use this as part of the model.

4.4. Calling Elements Positive if ≥k′ Out of k Bits are Positive.

One can generalize to a strategy where one calls a sample positive if ≥k′ out of the k corresponding bits are 1. The error rates for this scheme, with k′<k, are (Eq. 11)

$${FPP}_{Δ {k}^{′}} = {FPP}_{Δ} {+ \sum }_{i = {k}^{′}}^{k} ( \begin{matrix}k \\ i\end{matrix} ) {( {Δ}_{synth} + ( 1 - {Δ}_{synth} ) {( 1 - \frac{1 - {Δ}_{stoch}}{m} )}^{kn} )}^{i} {( ( 1 - {Δ}_{synth} ) ( 1 - {( 1 - \frac{1 - {Δ}_{stoch}}{m} )}^{kn} ) )}^{k - i} \qquad ( 11 )$$

$${FNP}_{Δ {k}^{′}} = {FNP}_{Δ} - {\sum }_{i = {k}^{′}}^{k} ( \begin{matrix}k \\ i\end{matrix} ) {( {Δ}_{synth} + ( 1 - {Δ}_{synth} ) {{Δ}_{stoch} ( 1 - \frac{1 - {Δ}_{stoch}}{m} )}^{k ( n - 1 )} )}^{k - i} {( ( 1 - {Δ}_{synth} ) ( 1 - {{Δ}_{stoch} ( 1 - \frac{1 - {Δ}_{stoch}}{m} )}^{k ( n - 1 )} ) )}^{i} .$$

derived in Section 8 of this Example (Math Appendix) along with exponential approximations and an upper bound FNP_{Δk′,max}.

Continuing to lower k′ leads to further drops in the false negative probability, with further gains in the false positive probability. Since the impact of k′=k−1 on the false positive probability was already possibly too much, this strategy is not very reasonable if one only has a single Bloom filter with b=100,000 and m=10,000 with low k. However, this becomes more useful given a second set of barcodes.

5. Adding a Second Barcode with No Redundancy.

There are many possible extensions if there is another orthogonal set of barcodes available. The number of barcodes in this orthogonal set can be denoted as m₂. Reasonable numbers for m₂ in real life are 10, 96, or 384. For example, samples could be distributed into 10 different batches, with a single one of the 10 orthogonal barcodes used for each batch. Alternatively, samples could be distributed into 96-well or 384-well plates, where each well could contain a specific orthogonal barcode that gets incorporated when Illumina adaptors are added for next-gen sequencing.

Critically, with current parameters m=100,000 and b=10,000, an additional 10 different orthogonal barcodes would already enable a unique assignment of (barcode 1, barcode 2) to each patient sample, but this is very challenging to implement in practice. The advantage of this Bloom filter approach is it allows for the pooling of samples as they come in, in a rolling manner. This approach is agnostic to exactly which barcodes a sample contains, as long as it is recorded.

5.1 Pooling into Non-Overlapping Sub-Batches, No First Barcodes.

Suppose that the samples were not initially barcoded with the scheme described above, and were only given the m₂ orthogonal barcodes/wells to dispense samples into. Then, assuming one can choose which samples go into which wells, this problem reduces to the initial problem of a single batch filter with m=m₂.

This leads to significantly elevated false positive probabilities, and a lower false negative probability. The Δ_{synth} term would correspond to some wells never working, even if it contained a positive sample, and the Δ_{stoch} term would correspond to a sample not getting added properly into a well or dropping out during downstream sequencing. So the results above are generalizable to other scenarios.

5.2 Pooling into Non-Overlapping Sub-Batches, with First Barcodes.

One potential strategy is to split the batch of b samples into m₂ non-overlapping sub-batches, using a single barcode of the m₂ orthogonal barcodes for each sub-batch. Then, each sub-batch can be modeled as an individual Bloom filter. That creates m₂ separate non-overlapping Bloom filters, each with a batch size of b₂=b/m₂.

As the other parameters do not change, one can compute the exact error probabilities for the three models for a single bloom filter by plugging in n₂=pb₂ for n into the formulas for FPP, FNP_{Δ}, FPP_{Δ}, FNP_{Δ2}, FPP_{Δ2}, FNP_{Δk′}, and FPP_{Δk′}.

Since the batch size has dropped by a factor of k₂, FPP_{Δ} will decrease. However, the FNP_{Δ,max} is not affected by varying the batch size, so this is not helpful towards the goal at hand.

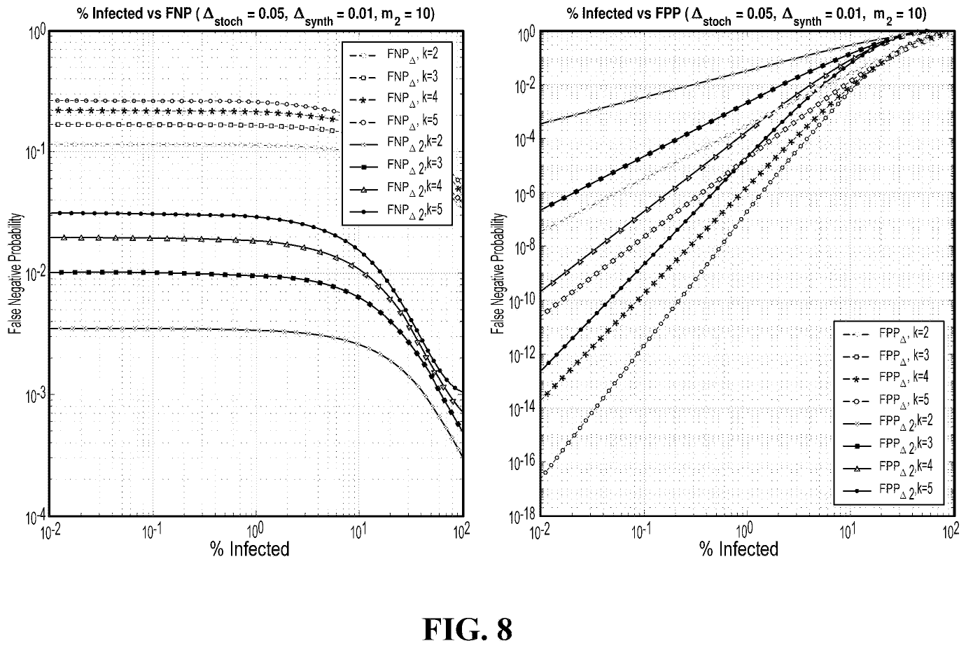

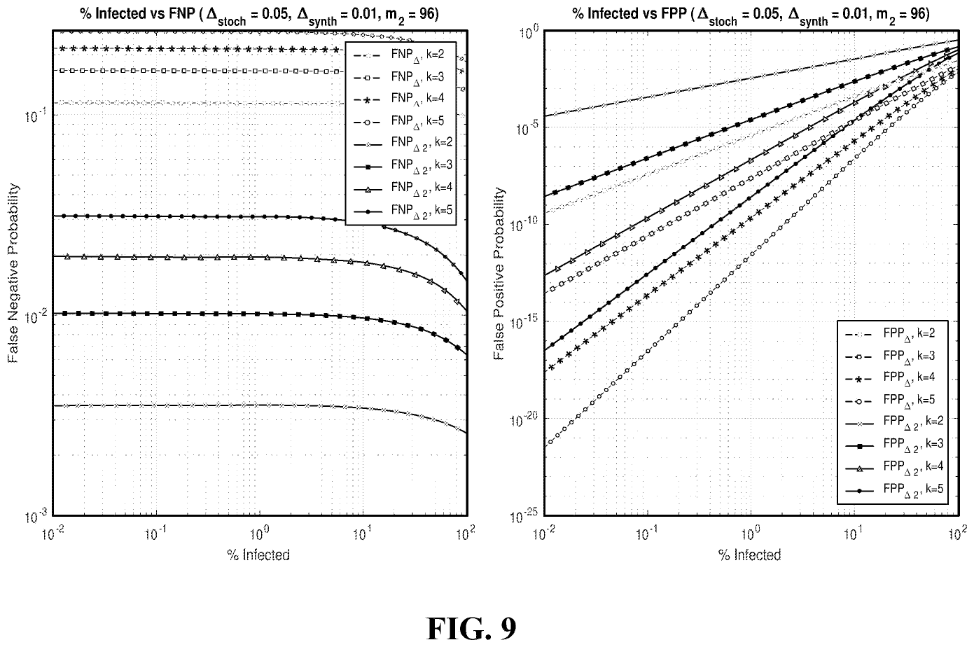

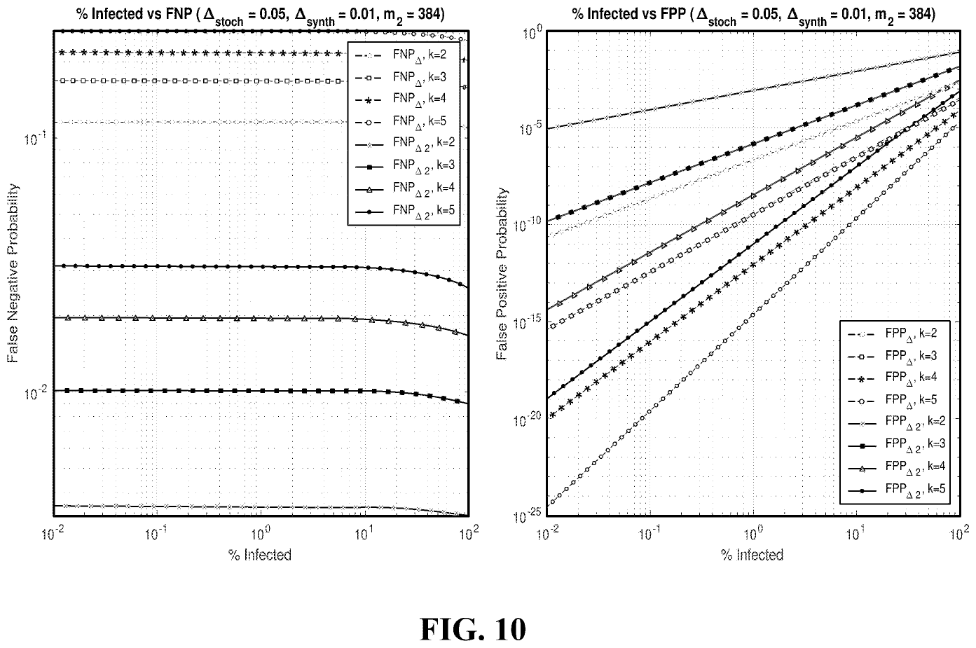

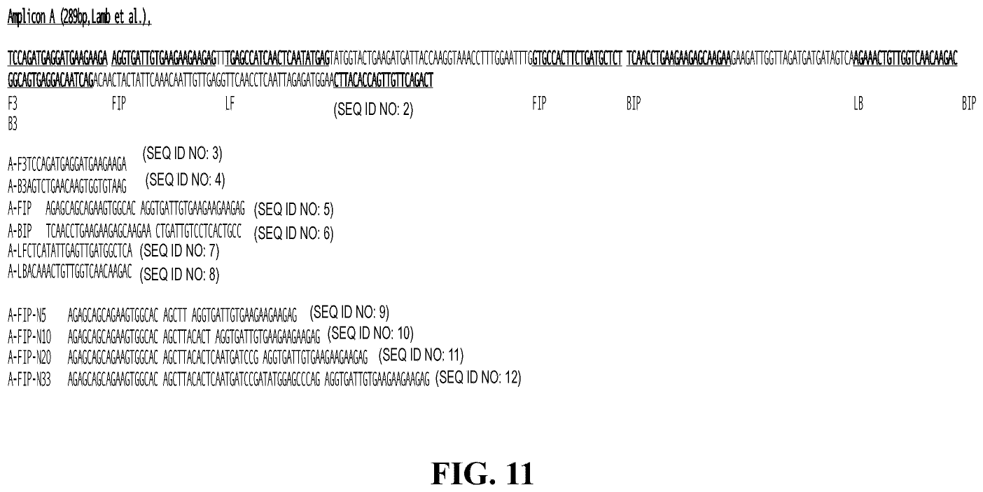

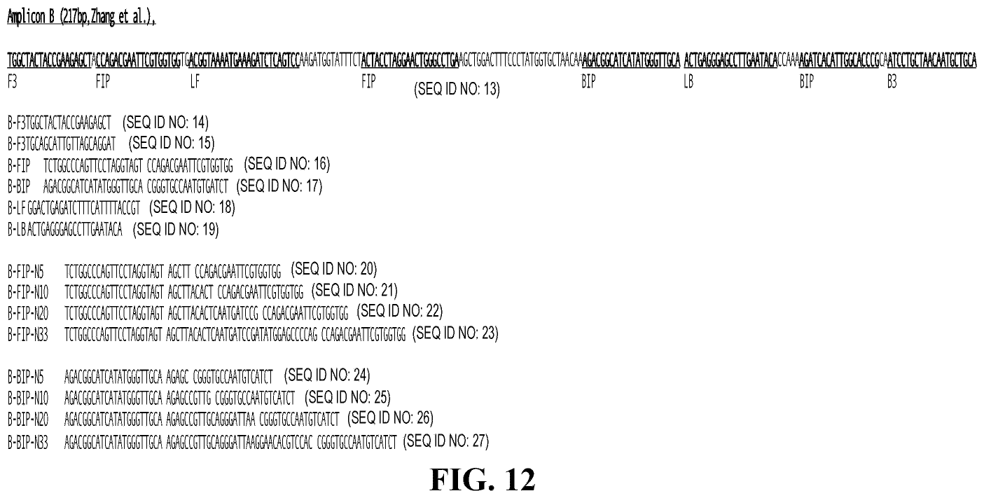

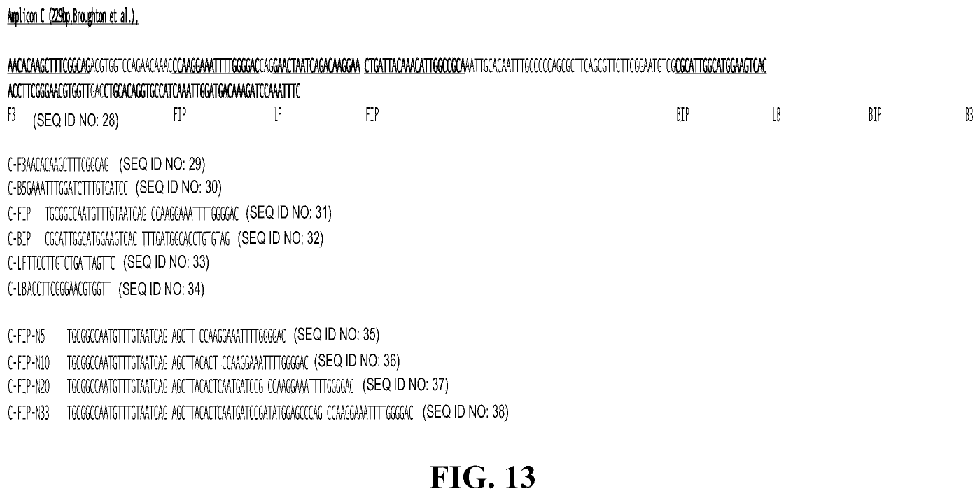

Alternatively, if one uses the ≥k−1 out of k model, the false positive probability can be reduced while retaining a perhaps reasonable false negative probability. The error rates for the three different m₂ values are shown in FIGS. 8-10, varying some other parameters. By utilizing these sub-batches, the FPP_{Δ2} is lowered to something much more reasonable, so one is able to make use of the lower FNP_{Δ2max}.

Perhaps the most reasonable approach is to use the ≥k′ out of k criterion. Then, plugging in n₂=n/m₂, the error rates are (Eq. 12)

$${FNP}_{Δ {k}^{′} , {m}_{2}} = 1 - {\sum }_{i = {k}^{′}}^{k} ( \begin{matrix}k \\ i\end{matrix} ) {( {Δ}_{synth} + ( 1 - {Δ}_{synth} ) {Δ}_{stoch} ( 1 - \frac{1 - {Δ}_{stoch}}{m} ) \text{?} )}^{k - i} {( ( 1 - {Δ}_{synth} ) ( 1 - {Δ}_{stoch} ( 1 - \frac{1 - {Δ}_{stoch}}{m} ) \text{?} ) )}^{i} . \qquad ( 12 )$$

$${FPP}_{Δ {k}^{′} , {m}_{2}} = {\sum }_{i = {k}^{′}}^{k} ( \begin{matrix}k \\ i\end{matrix} ) {( {Δ}_{synth} + ( 1 - {Δ}_{synth} ) ( 1 - \frac{1 - {Δ}_{stoch}}{m} ) \text{?} )}^{k - i} {( ( 1 - {Δ}_{synth} ) ( 1 - ( 1 - \frac{1 - {Δ}_{stoch}}{m} ) \text{?} ) )}^{i}$$

$$\text{?} \text{indicates text missing or illegible when filed}$$

Similarly, using the equations for the single Bloom filter with errors and the maximal FPP with no errors (Eq. 13A and 13B),

$${FNP}_{Δ {k}^{′} , {m}_{2} , max} = 1 - {\sum }_{i = {k}^{′}}^{k} ( \begin{matrix}k \\ i\end{matrix} ) {( {Δ}_{synth} + ( 1 - {Δ}_{synth} ) {Δ}_{stoch} )}^{k - i} {( ( 1 - {Δ}_{synth} ) ( 1 - {Δ}_{stoch} ) )}^{i} . \qquad ( 13 A , 13 B )$$

$${FPP}_{Δ {k}^{′} , {m}_{2} , max} = {\sum }_{i = {k}^{′}}^{k} ( \begin{matrix}k \\ i\end{matrix} ) {( ( 1 - \frac{1}{m} ) \text{?} )}^{k - i} {( 1 - ( 1 - \frac{1}{m} ) \text{?} )}^{i} .$$

$$\text{?} \text{indicates text missing or illegible when filed}$$

A few scenarios using this model are described in section 4 of this Example.

6. Redundancy with the Second Barcode.
6.1 Pooling into Overlapping Sub-Batches.

One other strategy would be to split the batch of b samples into m₂ sub-batches, and then dispense them into the m₂ orthogonally-barcoded wells in an overlapping fashion, so each well contains 2 different sub-batches. Then, each well will contain 2b/m₂ samples, and each sample will be represented twice. An example of this is to split all of the samples into 10 batches, and then add batches 1 and 2 into well 1, batches 2 and 3 into well 2, and so forth.

This redundancy allows for a reduction of the false positive rate, and for various new criteria for calling a sample positive. Each well again functions as a separate Bloom filter, but the direct mathematical analysis is hard here because the Bloom filters are no longer fully independent. Two wells containing a given sample have half of the samples that are the same.

Since this independence assumption is violated, the plan is to simulate this in Python using a vector/array in {0, 1}^{m} as the Bloom filter, and use the first k numbers out of a RNG between 1 and m with a seed i as the k barcodes for a given sample with index i. This should be a reasonable numerical simulation for this type of model, and hopefully not take too long to do.

### 6.2 Group Testing-Type Approach.

With a liquid handling robot, one could try to choose which wells a particular sample goes into. Taking a page from group testing, one way to use this to augment testing is to have k₂ wells for each sample, where the design matrix is chosen such that each sample ends up in a unique combination of k₂ wells. For now, let k₂=3. This is possible since 384 choose 3 and 96 choose 3 are both as big.

Since unique combinations were chosen, the samples in any two wells are ≈ independent. This means that, for each sample, there are now k₂ different single Bloom filters with barcode errors containing it, each with k₂b/m₂ samples. For b=100,000, k₂=3, m₂=384, this is ≈781 samples per well.

### 7. More Complex Scenarios.

If a patient sample can be distributed into more than one sub-batch, then one can introduce redundancy across sub-batches. There are many potential theoretical scenarios where one can leverage this redundancy to improve the inference process. However, this may be challenging to introduce in practice. Two of these scenarios, that have some potential for physical implementation, are described below.

7.1. Pooling into m₂ Half-Overlapping Sub-Batches.

One other strategy would be to split the batch of b samples into m₂ sub-batches, and then they are dispenses into the m₂ orthogonally-barcoded wells in an overlapping fashion, so each well contains 2 different sub-batches. Then, each well will contain 2b1 m₂ samples, and each sample will be represented twice. An example of this is to split all of the samples into 10 batches, and then add batches 1 and 2 into well 1, batches 2 and 3 into well 2, and so forth.

This redundancy allows for a reduction of the false positive rate, and for various new criteria for calling a sample positive. Each well again functions as a separate Bloom filter, but the direct mathematical analysis is difficult here as the Bloom filters are no longer fully independent. It is known that two wells containing a given sample have half of the samples that are the same. Since this independence assumption is violated, false negative and false positive probabilities may be best estimated for a given parameter set using numerical simulation.

7.2 Each Sample Goes into k₂ Random Wells.

Using a liquid handling robot, one could try to choose which wells a particular sample goes into. Taking a page from group testing, one way to use this to augment the testing is to have k₂ wells for each sample, where the design matrix is chosen such that each sample ends up in a unique combination of k₂ wells. For now, let k₂=3. This is possible since 384 choose 3 and 96 choose 3 are both big.

Since one has chosen unique combinations, the samples in any two wells are independent. This means that, for each sample, one now has k₂ different single Bloom filters with barcode errors containing it, each with k_{2b}/m₂ samples. For b=100,000, k₂=3, m₂=384, this is 781 samples per well.

Since the independence assumption is satisfied here, the error probabilities for a single one of these Bloom filters are the same as those computed in section 4.2, with a batch size of k_{2b}/m₂. This approach opens the doors to new criteria for calling a sample positive, such as calling a sample positive if at least k′ out of k barcodes are positive in 2 out of the 3 corresponding wells. The error rates for these kinds of approaches be computed using combinatorics with the error rates for a single one of these Bloom filters.

### 8. Math Appendix.

### 8.1. Single Bloom Filter, No Barcode Loss

### 8.1.1 Bloom Filter False Positive Probability.

Below is a derivation of the false positive probability of the Bloom filter. This makes certain assumptions about the hash functions at play, namely that they generate a uniform random distribution across all inputs, and that the hash functions are independent. This is basically true for the samples as one can assign the m unique barcodes across all samples randomly, although one would likely assign k different unique barcodes per sample which is not accounted for here.

The probability that a certain bit m′ is not set to 1 during the addition of a single element due to a certain hash function is 1-1/m. If the hash functions don't have a significant correlation (more or less valid), then, after all k hash functions have been used, this probability is (Eq. 14)

$$Pr [ {m}^{′} = 0 ] {( 1 - \frac{1}{m} )}^{k} \qquad ( 14 )$$

for a single element addition. If one now adds n total elements, then (Eq. 15)

$$Pr [ {m}^{′} = 0 ] = {( 1 - \frac{1}{m} )}^{kn} \qquad ( 15 )$$

$$and \qquad ( Eq . 16 )$$

$$Pr [ {m}^{′} = 1 ] = 1 - {( 1 - \frac{1}{m} )}^{kn} \qquad ( 16 )$$

Focusing on an element e that is not in the set: The k hash functions map it to k bits. So, the probability they are all 1, producing a false positive, is (Eq. 17)

$$Pr [ False Positive ] = {( 1 - {( 1 - \frac{1}{m} )}^{kn} )}^{k} \qquad ( 17 )$$

### 8.1.2. Optimal Choice of k.

Trying to solve for an optimal choice of k that minimizes the False Positive Probability for an ideal Bloom Filter: If one approximates (1-1/m)^{kn} as e-kn/m. This is a valid approximation for large m, and quite reasonable for an m of 1,0000. Then, for a fixed m and n, one would want (Eq. 18)

$${{min}_{k} ( 1 - {( 1 - \frac{1}{m} )}^{kn} )}^{k} \approx {{min}_{k} ( 1 - {e}^{- \frac{kn}{m}} )}^{k} \qquad ( 18 )$$

This is still ugly, so if one takes the natural log, giving (Eq. 19)

$${min}_{k} k ln ( 1 - {e}^{- \frac{kn}{m}} ) . \qquad ( 19 )$$

First order conditions give (Eq. 20)

$$ln ( 1 - {e}^{- \frac{kn}{m}} ) + \frac{kn}{m} \frac{{e}^{- \frac{kn}{m}}}{1 - {e}^{- \frac{kn}{m}}} = 0. \qquad ( 20 )$$

Let kn/m=ln x. Then, (Eq. 21)

$$\begin{matrix}0 = ln ( 1 - {e}^{- ln x} ) + ln x \frac{{e}^{- ln x}}{1 - {e}^{- ln x}} \\ = ln ( 1 - \frac{1}{x} ) + ln x \frac{\frac{1}{x}}{1 - \frac{1}{x}}\end{matrix} , \qquad ( 21 )$$

which solves to x=2. So, the optimal choice of k is (Eq. 22)

$${k}_{OPT} = ln ( 2 ) \frac{m}{n} . \qquad ( 22 )$$

At k_{OPT}, (Eq. 23)

$$\begin{matrix}{FPP}_{OPT} = {( 1 - {e}^{- {k}_{OPT} \frac{n}{m}} )}^{{k}_{OPT}} \\ = {( 1 - {e}^{- l n ( 2 )} )}^{{k}_{OPT}} \\ = {0.5}^{{k}_{OPT}}\end{matrix} . \qquad ( 23 )$$

### 8.1.3 Maximal Compression Factor.

One can also use the approximation to solve for a constraint on the b to m ratio given an error threshold E. Let c be the compression factor c=b/m. Then, (Eq. 24)

$${( 1 - {e}^{- \frac{kpb}{m}} )}^{k} \leq ϵ \qquad ( 24 )$$

$$or \qquad ( Eq . 25 )$$

$${e}^{- kpe} \geq 1 - {ϵ}^{\frac{1}{k}} \qquad ( 25 )$$

$$so \qquad ( Eq . 26 )$$

$$e \leq \frac{- ln ( 1 - {ϵ}^{\frac{1}{k}} )}{kp} \qquad ( 26 )$$

This c that is solved for can be interpreted as the number of samples per barcode that is run given the error threshold and some proportion p infected. So, for a given choice of p, k, and ε,

$$\begin{matrix}( Eq . 27 ) & \\ {e}_{max} = \frac{- ln ( 1 - {ϵ}^{\frac{1}{k}} )}{kp} . & ( 27 )\end{matrix}$$

Rearranging, one also obtains that, for a given choice of c, k, and ε, (Eq. 28)

$${p}_{max} = \frac{- ln ( 1 - {ϵ}^{\frac{1}{k}} )}{kc} . \qquad ( 28 )$$

8.4. Single Bloom Filter with Barcode Loss

Given the two types of errors introduced above, one can attempt to calculate the false positive and false negative probabilities. One will continue to assume that the hash functions are independent, so there is no accounting for the fact that the k barcodes for a given sample are probably all different (except for one step in the FNP derivation). This may mean that the error probabilities are too high.

For calculating the false positive probability: For a given bit m′, that at least functions some portion of the time, the probability it is not set to 1 during the addition of a single element due to a certain hash function is 1−(1−Δ_{stoch}) 1/m. After all k hash functions have been used, this probability is (Eq. 29)

$$Pr [ {m}^{′} = 0 ] = {( 1 - \frac{1 - {Δ}_{stoch}}{m} )}^{k} \qquad ( 29 )$$

for a single element addition. If one now adds u total elements, then (Eq. 30)

$$Pr [ {m}^{′} = 0 ] = {( 1 - \frac{1 - {Δ}_{stoch}}{m} )}^{kn} . \qquad ( 30 )$$

Incorporating the Δ_{synth} error (Eq. 31),

$$Pr [ {m}^{′} = 0 ] = {Δ}_{synth} + ( 1 - {Δ}_{synth} ) {( 1 - \frac{1 - {Δ}_{stoch}}{m} )}^{kn} \qquad ( 31 )$$

$$Pr [ {m}^{′} = 1 ] = ( 1 - {Δ}_{synth} ) ( 1 - {( 1 - \frac{1 - {Δ}_{stoch}}{m} )}^{kn} ) .$$

If one focuses on an element e that is not in the set: The k hash functions map it to k bits. So, the probability they are all 1, producing a false positive, is (Eq. 32)

$${FPP}_{Δ} = {( 1 - {Δ}_{synth} )}^{k} {( 1 - {( 1 - \frac{1 - {Δ}_{stoch}}{m} )}^{kn} )}^{k} . \qquad ( 32 )$$

If cleaned up a little, one can write (Eq. 33)

$$\begin{matrix}1 - {( 1 - \frac{1 - {Δ}_{stoch}}{m} )}^{kn} = 1 - {( {( 1 - \frac{1}{m / ( 1 - {Δ}_{stoch} )} )}^{m / ( 1 - {Δ}_{stoch} )} )}^{\frac{1 - {Δ}_{stoch}}{m} kn} \\ \approx 1 - {e}^{- ( 1 - {Δ}_{stoch} ) \frac{kn}{m}}\end{matrix} \qquad ( 33 )$$

for large m. This is a very close approximation for m=10,000. So, the false positive probability incorporating barcode errors, FPP_{Δ}, is approximately given by (Eq. 34)

$${FPP}_{Δ} \approx {( 1 - {Δ}_{synth} )}^{k} {( 1 - {e}^{- ( 1 - {Δ}_{stoch} ) \frac{kn}{m}} )}^{k} . \qquad ( 34 )$$

This checks out for the limiting cases of (Δ_{synth}=0, Δ_{stoch}=0), (Δ_{synth}=0, Δ_{stoch}=1), and (Δ_{synth}=1, Δ_{stoch}=0). Note that (Δ_{synth}=0, Δ_{stoch}=0) gives the original Bloom filter FPP, and the introduction of these errors lowers the FPP, so the original FPP is an upper bound.

One can now compute the false negative probability. Focusing on an element e′ that is in the set: The k hash functions map it to k bits. The probability of a false negative is 1−the probability it is called as positive, which requires all k bits to be positive. For a single one of these k bits m″, it will be 0 with probability (Eq. 35)

$$Pr [ {m}^{″} = 0 ] = {Δ}_{synth} + ( 1 - {Δ}_{synth} ) {{Δ}_{stoch} ( 1 - \frac{1 - {Δ}_{stoch}}{m} )}^{k ( n - 1 )} , \qquad ( 35 )$$

assuming that the k barcodes for e′ were different, which is realistic and makes the math much easier. Then, the probability that in m″ is 1 is just (Eq. 36)

$$Pr [ {m}^{″} = 1 ] = ( 1 - {Δ}_{synth} ) ( 1 - {{Δ}_{stoch} ( 1 - \frac{1 - {Δ}_{stoch}}{m} )}^{k ( n - 1 )} ) . \qquad ( 36 )$$

Thus, the false negative probability incorporating barcode errors, FNP_{Δ}, is Eq. 37, Eq. 38)

$$\begin{matrix}{FNP}_{Δ} = 1 - {( 1 - {Δ}_{synth} )}^{k} {( 1 - {{Δ}_{stoch} ( 1 - \frac{1 - {Δ}_{stoch}}{m} )}^{k ( n - 1 )} )}^{k} \\ \approx 1 - {( 1 - {Δ}_{synth} )}^{k} {( 1 - {Δ}_{stoch} {e}^{- ( 1 - {Δ}_{stoch} ) \frac{k ( n - 1 )}{m}} )}^{k}\end{matrix} \qquad ( 37 , 38 )$$

by the same approximation as above

Note that there is a dependence on the number of people infected n in a given batch, due to the possibility of some other positive samples compensating for the dropped out barcode(s) for a given positive sample. One can produce an upper-bound on this probability, FNP_{Δmax}, by not allowing for this compensation, giving (Eq. 39)

FNP_{Δ,max}=1−(1−Δ_{synth})^{k}(1−Δ_{stoch})^{k} (39)

8.5. Positives Given as ≥k−1 Out of k Barcodes Positive.

It is now established that for a random given bit m′, after n elements have been inserted (Eq. 40),

$$Pr [ {m}^{′} = 0 ] = {Δ}_{synth} + ( 1 - {Δ}_{synth} ) {( 1 - \frac{1 - {Δ}_{stoch}}{m} )}^{kn} \qquad ( 40 )$$

$$Pr [ {m}^{′} = 1 ] = ( 1 - {Δ}_{synth} ) ( 1 - {( 1 - \frac{1 - {Δ}_{stoch}}{m} )}^{kn} ) .$$

If one now focuses on an element e that is not in the set, the k hash functions map it to k bits. Under this model, at least k−1 bits have to be 1, so, the probability of a false positive (abusing notation), is (Eq. 41)

$$\begin{matrix}Pr [ False Positive ] = {Pr [ {m}^{′} = 1 ]}^{k} + kPr [ {m}^{′} = 0 ] {Pr [ {m}^{′} = 1 ]}^{k - 1} \\ = {FPP}_{Δ} + kPr [ {m}^{′} = 0 ] {Pr [ {m}^{′} = 1 ]}^{k - 1}\end{matrix} . \qquad ( 41 )$$

$$So , \qquad ( Eq . 42 )$$

$$\begin{matrix}{FPP}_{Δ 2} = {FPP}_{Δ} + k ( {Δ}_{synth} + ( 1 - {Δ}_{synth} ) {( 1 - \frac{1 - {Δ}_{stoch}}{m} )}^{kn} ) \\ {( ( 1 - {Δ}_{synth} ) ( 1 - {( 1 - \frac{1 - {Δ}_{stoch}}{m} )}^{kn} ) )}^{k - 1} \\ = {( ( 1 - {Δ}_{synth} ) ( 1 - {( 1 - \frac{1 - {Δ}_{stoch}}{m} )}^{kn} ) )}^{k} \\ + k ( {Δ}_{synth} + ( 1 - {Δ}_{synth} ) {( 1 - \frac{1 - {Δ}_{stoch}}{m} )}^{kn} ) \\ {( ( 1 - {Δ}_{synth} ) ( 1 - {( 1 - \frac{1 - {Δ}_{stoch}}{m} )}^{kn} ) )}^{k - 1}\end{matrix} \qquad ( 42 )$$

Using the approximation (Eq. 43),

$$\begin{matrix}{FPP}_{Δ 2} \approx {FPP}_{Δ} + k ( {Δ}_{synth} + ( 1 - {Δ}_{synth} ) {e}^{- ( 1 - {Δ}_{stoch} ) \frac{k ( n - 1 )}{m}} ) \\ {( ( 1 - {Δ}_{synth} ) ( 1 - {e}^{- ( 1 - {Δ}_{stoch} ) \frac{k ( n - 1 )}{m}} ) )}^{k - 1} \\ \approx {( ( 1 - {Δ}_{synth} ) ( 1 - {e}^{- ( 1 - {Δ}_{stoch} ) \frac{kn}{m}} ) )}^{k} + k ( {Δ}_{synth} + ( 1 - {Δ}_{synth} ) {e}^{- ( 1 - {Δ}_{stoch} ) \frac{k ( n - 1 )}{m}} ) \\ {( ( 1 - {Δ}_{synth} ) ( 1 - {e}^{- ( 1 - {Δ}_{stoch} ) \frac{k ( n - 1 )}{m}} ) )}^{k - 1}\end{matrix} . \qquad ( 43 )$$

One will now compute the false negative probability. Focusing on an element e′ that is in the set: The k hash functions map it to k bits. The probability of a false negative is 1−the probability it is called as positive, which requires ≥k−1 bits to be positive. For a single one of these k bits m″, one gets (Eq. 44)

$$Pr [ {m}^{″} = 0 ] = {Δ}_{synth} + ( 1 - {Δ}_{synth} ) {{Δ}_{stoch} ( 1 - \frac{1 - {Δ}_{stoch}}{m} )}^{k ( n - 1 )} \qquad ( 44 )$$

$$Pr [ {m}^{″} = 1 ] = ( 1 - {Δ}_{synth} ) ( 1 - {{Δ}_{stoch} ( 1 - \frac{1 - {Δ}_{stoch}}{m} )}^{k ( n - 1 )} ) .$$

So, the false negative probability incorporating barcode errors, FNP_{Δ2}, is (Eq. 45)

$$\begin{matrix}{FNP}_{Δ 2} = 1 - {Pr [ {m}^{″} = 1 ]}^{k} - k Pr [ {m}^{″} = 0 ] {Pr [ {m}^{″} = 1 ]}^{k - 1} \\ = {FNP}_{Δ} - kPr [ {m}^{″} = 0 ] {Pr [ {m}^{″} = 1 ]}^{k - 1}\end{matrix} . \qquad ( 45 )$$

$$Expanding , \qquad ( Eq . 46 )$$

$$\begin{matrix}{FNP}_{Δ 2} = {FNP}_{Δ} - k ( {Δ}_{synth} + ( 1 - {Δ}_{synth} ) {{Δ}_{stoch} ( 1 - \frac{1 - {Δ}_{stoch}}{m} )}^{k ( n - 1 )} ) \\ {( ( 1 - {Δ}_{synth} ) ( 1 - {{Δ}_{stoch} ( 1 - \frac{1 - {Δ}_{stoch}}{m} )}^{k ( n - 1 )} ) )}^{k - 1} \\ = 1 - {( 1 - {Δ}_{synth} )}^{k} {( 1 - {{Δ}_{stoch} ( 1 - \frac{1 - {Δ}_{stoch}}{m} )}^{k ( n - 1 )} )}^{k} \\ - k ( {Δ}_{synth} + ( 1 - {Δ}_{synth} ) {{Δ}_{stoch} ( 1 - \frac{1 - {Δ}_{stoch}}{m} )}^{k ( n - 1 )} ) \\ {( ( 1 - {Δ}_{synth} ) ( 1 - {{Δ}_{stoch} ( 1 - \frac{1 - {Δ}_{stoch}}{m} )}^{k ( n - 1 )} ) )}^{k - 1}\end{matrix} . \qquad ( 46 )$$

Using the approximation (Eq. 47),

$$\begin{matrix}{FNP}_{Δ 2} \approx {FNP}_{Δ} - k ( {Δ}_{synth} + ( 1 - {Δ}_{synth} ) {Δ}_{stoch} {e}^{- ( 1 - {Δ}_{stoch} ) \frac{k ( n - 1 )}{m}} ) \\ {( ( 1 - {Δ}_{synth} ) ( 1 - {Δ}_{stoch} {e}^{- ( 1 - {Δ}_{stoch} ) \frac{k ( n - 1 )}{m}} ) )}^{k - 1} \\ \approx 1 - {( 1 - {Δ}_{synth} )}^{k} {( 1 - {Δ}_{stoch} {e}^{- ( 1 - {Δ}_{stoch} ) \frac{k ( n - 1 )}{m}} )}^{k} \\ - k ( {Δ}_{synth} + ( 1 - {Δ}_{synth} ) {{Δ}_{stoch} ( 1 - \frac{1 - {Δ}_{stoch}}{m} )}^{k ( n - 1 )} ) \\ {( ( 1 - {Δ}_{synth} ) ( 1 - {{Δ}_{stoch} ( 1 - \frac{1 - {Δ}_{stoch}}{m} )}^{k ( n - 1 )} ) )}^{k - 1}\end{matrix} . \qquad ( 47 )$$

Applying the same logic above, one can compute an upper bound on this probability (Eq. 48)

$$\begin{matrix}{FNP}_{Δ 2 , max} = {FNP}_{Δ , max} - k ( {Δ}_{synth} + ( 1 - {Δ}_{synth} ) {Δ}_{stoch} ) {( ( 1 - {Δ}_{synth} ) ( 1 - {Δ}_{stoch} ) )}^{k - 1} \\ = 1 - {( 1 - {Δ}_{synth} )}^{k} {( 1 - {Δ}_{stoch} e )}^{k} \\ - k ( {Δ}_{synth} + ( 1 - {Δ}_{synth} ) {Δ}_{stoch} ) {( ( 1 - {Δ}_{synth} ) ( 1 - {Δ}_{stoch} ) )}^{k - 1}\end{matrix} . \qquad ( 48 )$$

Positives Given as ≥k′ Out of k Barcodes Positive

First, the false positive probability is computed. Let's again focus on an element e that is not in the set. The k hash functions map it to k bits. Under this model, at least k′ bits have to be 1, so, the probability of a false positive, is (Eq. 53)

$$\begin{matrix}{FPP}_{Δ {k}^{′}} = {\sum }_{i = {k}^{′}}^{k} ( \begin{matrix}k \\ i\end{matrix} ) {Pr [ {m}^{′} = 1 ]}^{i} {Pr [ {m}^{′} = 0 ]}^{k - i} \\ = {FPP}_{Δ} + {\sum }_{i = {k}^{′}}^{k - 1} ( \begin{matrix}k \\ i\end{matrix} ) {Pr [ {m}^{′} = 1 ]}^{i} {Pr [ {m}^{′} = 0 ]}^{k - i}\end{matrix} \qquad ( 53 )$$

$$for {k}^{′} < k .$$

$$So , \qquad ( Eq . 54 )$$

$$\begin{matrix}{FPP}_{Δ {k}^{′}} = {\sum }_{i = {k}^{′}}^{k} ( \begin{matrix}k \\ i\end{matrix} ) {( {Δ}_{synth} + ( 1 - {Δ}_{synth} ) {( 1 - \frac{1 - {Δ}_{stoch}}{m} )}^{kn} )}^{k - i} \\ {( ( 1 - {Δ}_{synth} ) ( 1 - {( 1 - \frac{1 - {Δ}_{stoch}}{m} )}^{kn} ) )}^{i} \\ = {FPP}_{Δ} + {\sum }_{i = {k}^{′}}^{k - 1} ( \begin{matrix}k \\ i\end{matrix} ) {( {Δ}_{synth} + ( 1 - {Δ}_{synth} ) {( 1 - \frac{1 - {Δ}_{stoch}}{m} )}^{kn} )}^{k - i} \\ {( ( 1 - {Δ}_{synth} ) ( 1 - {( 1 - \frac{1 - {Δ}_{stoch}}{m} )}^{kn} ) )}^{i}\end{matrix} . \qquad ( 54 )$$

Using the approximation (Eq. 55)

$$\begin{matrix}{FPP}_{Δ {k}^{′}} \approx {FPP}_{Δ} + {\sum }_{i = {k}^{′}}^{k - 1} ( \begin{matrix}k \\ i\end{matrix} ) {( {Δ}_{synth} + ( 1 - {Δ}_{synth} ) {e}^{- ( 1 - {Δ}_{stoch} ) \frac{k ( n - 1 )}{m}} )}^{k - i} \\ {( ( 1 - {Δ}_{synth} ) ( 1 - {e}^{- ( 1 - {Δ}_{stoch} ) \frac{k ( n - 1 )}{m}} ) )}^{i} \\ \approx {\sum }_{i = {k}^{′}}^{k} ( \begin{matrix}k \\ i\end{matrix} ) {( {Δ}_{synth} + ( 1 - {Δ}_{synth} ) {e}^{- ( 1 - {Δ}_{stoch} ) \frac{k ( n - 1 )}{m}} )}^{k - i} \\ {( ( 1 - {Δ}_{synth} ) ( 1 - {e}^{- ( 1 - {Δ}_{stoch} ) \frac{k ( n - 1 )}{m}} ) )}^{i}\end{matrix} . \qquad ( 55 )$$

The false negative probability is now computed. Let's focus on an element e′ that is in the set. The k hash functions map it to k bits. The probability of a false negative is 1−(the probability it is called as positive), which requires ≥k′ bits to be positive. So, the false negative probability incorporating barcode errors, FNP_{Δ2}, is (Eq. 56)

$$\begin{matrix}{FNP}_{Δ {k}^{′}} = 1 - {\sum }_{i = {k}^{′}}^{k} ( \begin{matrix}k \\ i\end{matrix} ) {Pr [ {m}^{″} = 1 ]}^{i} {Pr [ {m}^{″} = 0 ]}^{k - 1} \\ = {FNP}_{Δ} - {\sum }_{i = {k}^{′}}^{k - 1} ( \begin{matrix}k \\ i\end{matrix} ) {Pr [ {m}^{″} = 1 ]}^{i} {Pr [ {m}^{″} = 0 ]}^{k - 1}\end{matrix} \qquad ( 56 )$$

$$for {k}^{′} < k . Expanding ,$$

$$\begin{matrix}{FNP}_{Δ {k}^{′}} = 1 - {\sum }_{i = {k}^{′}}^{k} ( \begin{matrix}k \\ i\end{matrix} ) {( {Δ}_{synth} + ( 1 - {Δ}_{synth} ) {{Δ}_{stoch} ( 1 - \frac{1 - {Δ}_{stoch}}{m} )}^{k ( n - 1 )} )}^{k - i} \\ {( ( 1 - {Δ}_{synth} ) ( 1 - {{Δ}_{stoch} ( 1 - \frac{1 - {Δ}_{stoch}}{m} )}^{k ( n - 1 )} ) )}^{i} \\ = {FNP}_{Δ} - {\sum }_{i = {k}^{′}}^{k} ( \begin{matrix}k \\ i\end{matrix} ) {( {Δ}_{synth} + ( 1 - {Δ}_{synth} ) {{Δ}_{stoch} ( 1 - \frac{1 - {Δ}_{stoch}}{m} )}^{k ( n - 1 )} )}^{k - i} \\ {( ( 1 - {Δ}_{synth} ) ( 1 - {{Δ}_{stoch} ( 1 - \frac{1 - {Δ}_{stoch}}{m} )}^{k ( n - 1 )} ) )}^{i}\end{matrix} .$$

Using the approximation (Eq. 57)

$$\begin{matrix}{FNP}_{Δ {k}^{′}} \approx {FNP}_{Δ} - {\sum }_{i = {k}^{′}}^{k - 1} ( \begin{matrix}k \\ i\end{matrix} ) {( {Δ}_{synth} + ( 1 - {Δ}_{synth} ) {Δ}_{stoch} {e}^{- ( 1 - {Δ}_{stoch} ) \frac{k ( n - 1 )}{m}} )}^{k - i} \\ {( ( 1 - {Δ}_{synth} ) ( 1 - {Δ}_{stoch} {e}^{- ( 1 - {Δ}_{stoch} ) \frac{k ( n - 1 )}{m}} ) )}^{i} \\ \approx 1 - {\sum }_{i = {k}^{′}}^{k} ( \begin{matrix}k \\ i\end{matrix} ) {( {Δ}_{synth} + ( 1 - {Δ}_{synth} ) {Δ}_{stoch} {e}^{- ( 1 - {Δ}_{stoch} ) \frac{k ( n - 1 )}{m}} )}^{k - i} \\ {( ( 1 - {Δ}_{synth} ) ( 1 - {Δ}_{stoch} {e}^{- ( 1 - {Δ}_{stoch} ) \frac{k ( n - 1 )}{m}} ) )}^{i}\end{matrix} . \qquad ( 57 )$$

Applying the same logic as above, an upper bound can be computed on this probability (Eq. 58)

$$\begin{matrix}{FNP}_{Δ {k}^{′} , max} = {FNP}_{Δ , max} {\sum }_{i = {k}^{′}}^{k - 1} ( \begin{matrix}k \\ i\end{matrix} ) {( {Δ}_{synth} + ( 1 - {Δ}_{synth} ) {Δ}_{stoch} )}^{k - i} \\ {( ( 1 - {Δ}_{synth} ) ( 1 - {Δ}_{stoch} ) )}^{i} \\ = 1 - {\sum }_{i = {k}^{′}}^{k} ( \begin{matrix}k \\ i\end{matrix} ) {( {Δ}_{synth} + ( 1 - {Δ}_{synth} ) {Δ}_{stoch} )}^{k - i} \\ {( ( 1 - {Δ}_{synth} ) ( 1 - {Δ}_{stoch} ) )}^{i}\end{matrix} . \qquad ( 58 )$$

### Example 4—Proposed Design of a Privacy-Aware Web App for Delivering Diagnostic Results

### Step 1: Creating a Personal ID

1. The user opens a specific website on a computer,
2. The website displays a high-complexity pseudo-random QR code,
3. The user prints the QR code, which serves as an anonymous user ID,
4. If the user has no ability to print, a family member, nurse, or social worker can supply the user with a printed ID code.

### Step 2: Making an Appointment for a Swab

1. The user uses the camera of a smartphone to scan the printed ID code,
2. It opens a link to a website, which displays an available appointment slot of 15 minutes and a location,
3. Optionally, the user's OPS coordinates or randomly blurred OPS coordinates are used to spatially balance the population tested,
4. The user either reserves the slot, or requests an alternative slot

### Step 3: Getting a Swab Taken

1. The user waits in line with 2 m distancing from others,
2. A medical worker scans the user's ID code as the QR code of a reagent plate,
3. A swab is taken, inserted into the reagent plate, and processed further.

### Step 4: Sequencing Data Analysis

1. Sequencing data are analyzed and stored on a server, linked to the user's ID code,
2. No personal information or means of identification are recorded.

### Step 5: Retrieving the Result

1. The user uses the camera of a smartphone to scan the printed ID code,
2. It opens a link to a website, which displays the test result along with further instructions,
3. According to law, the user might be obligated to report a positive result to a public health authority.

### Example 5

As of May 2020, the recent global spread of a novel coronavirus, SARS-CoV-2, has already resulted in over 5,900,000 confirmed cases and 364,000 deaths (Johns Hopkins CSEE Covid tracker (Dong et al., 2020)) since its emergence several months ago. Early epidemiological studies indicate that the exponential spread of COVID-19, the disease caused by SARS-CoV-2, can be slowed by restrictive isolation measures (Chinazzi et al., 2020), but these measures place an enormous burden on societies and economies. Moreover, once isolation measures are lifted, exponential spread is predicted to resume (Li et al., 2020). To exacerbate the problem, many infected individuals do not show symptoms, are unlikely to seek testing, and unknowingly contribute to the spread of infection. To effectively combat the transmission of SARS-CoV-2, we need population-scale testing that can enable rapid identification of all infected individuals, pinpoint key potential outbreak areas to support epidemiological interventions that maximize human health, while reducing the extent of restrictive isolation measures (Taipale et al., 2020).

Currently, the majority of COVID-19 testing is performed using viral RNA extraction followed by RT-qPCR to amplify and detect one of several highly conserved regions of the SARS-CoV-2 genome, or by detecting serum antibodies specific for viral proteins. The global capacity for testing using these approaches, however, has been limited by a combination of access and supply issues for reagents and instruments.

Described here is LAMP-Seq, a protocol that allows for population-scale testing using massively parallel RT-LAMP (Nagamine et al., 2002; Notomi et al., 2000) by employing sample-specific barcodes. This approach requires only two heating steps for each individual sample (i.e., oropharyngeal swab, nasal swab, nasal wash, fecal sample, or potentially sputum), followed by pooled processing, parallelizable deep sequencing, and custom computational analysis. By using a simple thermal protocol for individual samples and pooling many samples prior to resource-intensive steps, the requirement for specialized reagents, equipment, and labor is greatly reduced as compared to established RT-qPCR protocols. Unique tracking of tens of millions of samples as well as asynchronous testing logistics can be achieved by employing a compressed barcode space. Herein the design and initial validation of the method is described as well as simulated barcoding strategies. It is estimated that the cost per sample would be <20 USD based on the list-price of off-the-shelf products (exclusive of labor and instrument costs), with a potential for a further 10-fold cost reduction through optimized production of two enzymes. Most importantly, this approach is predicted to be scalable to hundreds of thousands of samples per day per sequencing facility.

### Method Design

Described herein is the following approach for population-scale testing for SARS-CoV-2 infection: a barcoded RT-LAMP reaction is performed on an unpurified or lysed swab sample with primers specific for the SARS-CoV-2 genome, which is followed by large-scale pooling of samples, PCR amplification with additional barcoding, deep sequencing, and data analysis to identify positive individuals (FIGS. 1A-1B) (see below for detailed protocol). RT-LAMP reactions have been demonstrated to be highly sensitive for sequence-specific viral nucleic acid detection (Lamb et al., 2020; Yang et al., 2020; Zhang et al., 2020), even from unpurified samples (Estrela et al., 2019). To establish a barcoded RT-LAMP reaction, Applicant inserted barcode sequences into the forward inner primer (FIP), which enables generation of repeatedly barcoded palindromic amplification products (FIG. 1C). To limit the number of unique barcode primers needed for testing a large number of samples, a compressed barcode space can be used (see below for details) as long as a small fraction of samples is expected to be positive during population scale testing.

### Results

Applicant designed three barcoded primer sets based on validated RT-LAMP amplicons ((Broughton et al., 2020; Lamb et al., 2020; Zhang et al., 2020)) perfectly matching 97.5% (amplicon A), 96.9% (amplicon B), and 95.6% (amplicon C) of 183 SARS-CoV-2 genomes available in the NCBI database (Apr. 1, 2020). 10-nt barcodes with GC content of 30%-70% and no homopolymer repeats of more than four nucleotides were inserted into the FIP primer. Barcodes were made robust to sequencing errors by ensuring a minimum Levenshtein edit distance between any barcode pair sufficient to detect either one (10,000 barcode set) or two (1,000 barcode set) insertion, deletion or substitution errors.

Comparing barcoded LAMP reactions to non-barcoded controls using a dsDNA surrogate template for SARS-CoV-2, Applicant confirmed that the presence of a prototypical 10-nt barcode within the FIP primer did not affect LAMP sensitivity, product amounts, or downstream PCR amplification (FIG. 1D). Templating two individually barcoded LAMP reactions with 100-fold differing amounts of dsDNA template, combining them for PCR amplification, and sequencing the products resulted in read numbers within a two-fold range between the two samples (FIG. 1E), indicating that RT-LAMP saturation can effectively compress the dynamic range of input viral loads, potentially enabling higher multiplexing. Furthermore, the expected sequence of barcoded RT-LAMP-PCR products was confirmed by Next-Generation Sequencing (FIG. 1F). In order to determine the molecular sensitivity of barcoded RT-LAMP reactions, 24 reactions were templated with differing numbers of template RNA molecules. The number of positive subsequent PCR reactions was determined using gel electrophoresis (FIG. 1G). Fitting the fractions of positive reactions to a Poisson-distributed model of RNA detection, we estimated the molecular detection efficiency to be 1/37 per RNA molecule (FIG. 111).

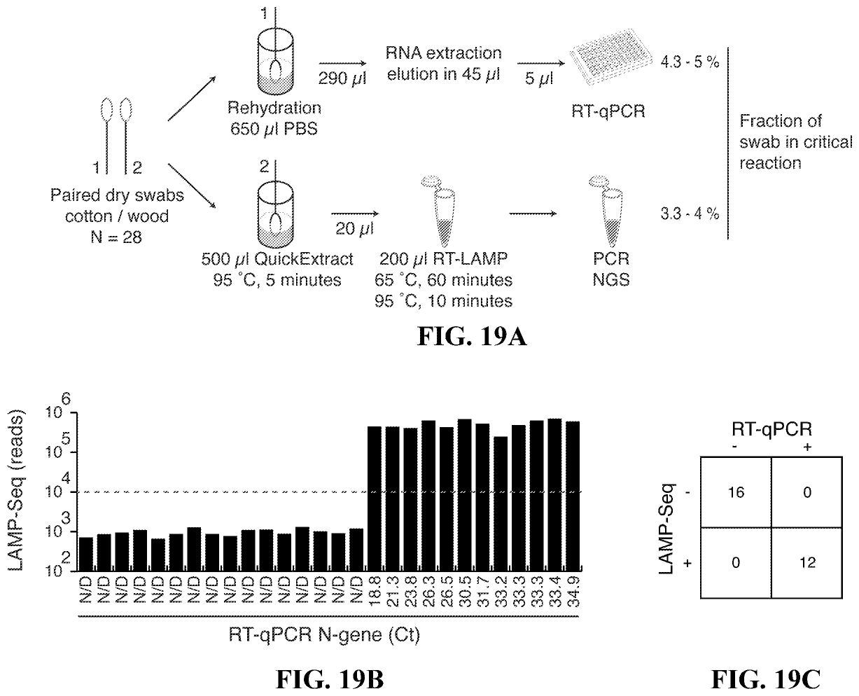

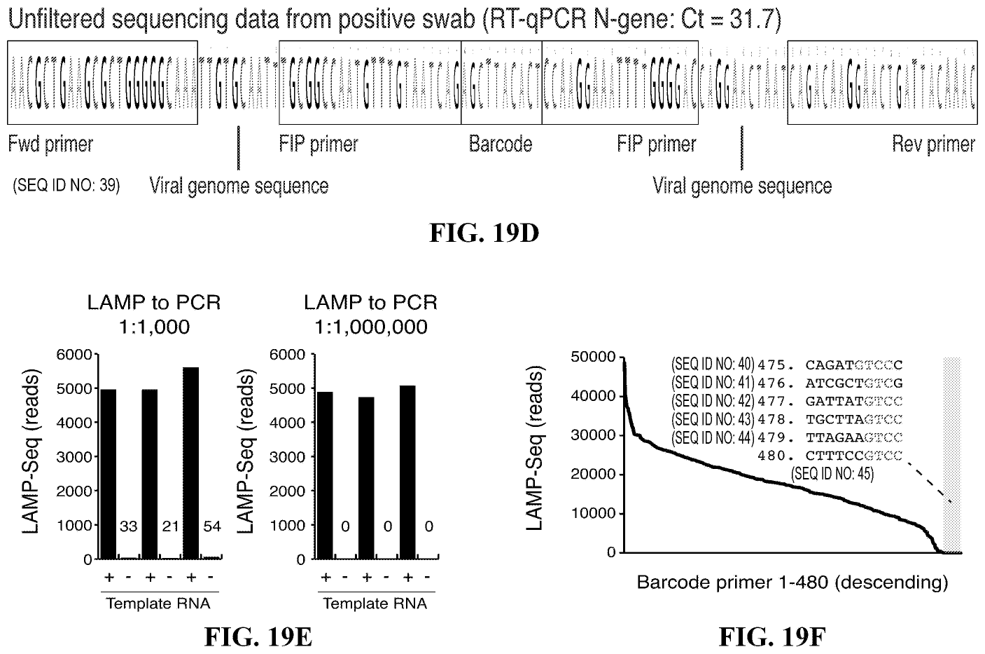

LAMP-Seq was tested on 28 clinical samples employing the following protocol: Upon informed consent, which was obtained as approved by the ethics committee of the University Hospital Bonn, two Oropharyngeal samples were collected from each individual using two separate cotton swabs, which were anonymized using an individual ID. One swab was analyzed using a standard clinical pipeline comprising of rehydration, robotic RNA purification, and RT-qPCR (FIG. 19A, upper panel). The other swab was immediately inserted into a tube containing QuickExtract lysis buffer (FIG. 19A, lower panel), processed and sequenced according to the LAMP-Seq protocol not employing pooling at the RT-LAMP stage (see Materials and Methods section). All of 12 individuals identified as SARS-CoV-2 RNA positive by RT-qPCR were detected positive using LAMP-Seq employing a threshold of 100,000 reads; the remaining 16 individuals were identified as negative for viral RNA in agreement between both methods (FIGS. 19B-19C). Unfiltered LAMP-Seq sequencing data confirmed the expected read structure, comprising primer sequences, stretches of viral genome sequence, and a barcode (FIG. 19D).

Pooling of barcoded RT-LAMP reactions will be essential for scaling up LAMP-Seq. When pooling six barcoded RT-LAMP reactions of which three had been templated with RNA, and performing PCR and sequencing, we observed significant levels of barcode swapping (FIG. 19A, left panel). Hypothesizing that barcode primers being transferred into the PCR reaction may engage in amplification and re-barcoding of amplicons, we tested diluting pooled RT-LAMP reactions one-million-fold in the PCR reaction, which indeed eliminated detectable barcode swapping (FIG. 19E, right panel).

As a prerequisite for establishing a scalable LAMP-Seq diagnostics pipeline, a large number of barcode primers have to be individually validated. Pooling 480 barcoded FIP primers, performing RT-LAMP reactions in four replicates, and sequencing the barcode distribution in resulting products revealed that a significant fraction of barcode sequences performs poorly or even fails to engage in LAMP-Seq (FIG. 19F). Investigating potential sequence determinants that could guide optimized barcode design, Applicant observed that the least efficient barcode primers displayed a marked enrichment for a GTCC motif or truncations thereof, preferentially towards the 3′ end of the barcode (FIG. 19F, inlet). As this motif is the reverse complement of the 3′ end of the FIP primer, Applicant estimates it could sequester the 3′ end by forming as intramolecular structure, thus hindering elongation of the primer, and should be avoided.

A high-output Illumina NextSeq run can routinely generate 200 million sequencing reads in 14 hours, which is predicted to be sufficient for 100,000 patient samples per run, even accounting for library skewing due to differences in viral loads, largely because the vast majority of samples will be negative. Barcoding 100,000 samples could be achieved by a naïve approach, where each sample gets a unique combination of a LAMP barcode and PCR barcode (FIG. 20A, left panel). Applicant named this scenario synchronous testing, because it assumes the barcodes to be reused and to be fully defined, so that barcoding either has to take place at a central location, or barcoded samples have to be shipped on an exact schedule. In remote-testing scenarios according to FIG. 1B, however, it cannot be ensured that barcoded samples to arrive in a predictable fashion (asynchronous testing), in which case it would be advantageous if every citizen who might ever be tested could be assigned a unique molecular barcode. Under this assumption, barcoded samples arriving on a given day could be processed in a rolling manner, offering more flexible logistics while barcoding remotely.

As synthesis, validation, and robotic handling of a unique primer for each citizen would hardly be feasible, a compressed barcode space was explored, where every sample would be assigned a unique combination of more than one barcode. For that, it was conservatively assumed that 1% of synthesized barcode primers systematically fail to work (Δ_{synth}=0.01), while additionally 5% of all sample-specific barcodes are not detected due to varying sequencing depth (Δ_{stoch}=0.05; this is independent of dropout due to low viral load). For automated assembly of testing reactions with unique barcode combinations, it was anticipated that m=1,000 barcode primers can be easily handled by available pipetting robots. Under these assumptions, 100,000 samples were investigated for what number of barcodes per sample (k), number of barcode primers total (m), and number of pools per run (m₂) would allow for minimal false-positive and false-negative rates of detection (FIGS. 20B-20C). Interpreting the compressed barcoding problem as a modified Bloom filter, it was predicted that when using k=5 barcodes per sample, requesting k′=3 barcodes to be detected per sample, and splitting samples into m₂=10 pools per run, both the false-negative and false-positive rates of detection using a compressed barcode space will be less than 0.2% as long as the global frequency of positive samples is below 1.2% (FIG. 19A). Larger numbers of barcodes will further lower error rates and enable higher global positive frequencies.

Since some barcoded FIP primers fail in the RT-LAMP reaction, it may be advantageous to use a smaller number of barcoded primers that can each be individually validated. One way to achieve this is with a dual barcoding scheme, where both the FIP and BIP primers are barcoded (FIG. 20D). Without compression, 100,000 patient samples could be uniquely barcoded using 100 FIP primers, 100 BIP primers, and 10 pools per run. With compression, over 20 billion samples can each be assigned a unique combination of barcodes using a combination of 3 FIP primers and a combination of 3 BIP primers per patient sample from a pool of 96 barcoded FIP and 96 barcoded BIP primers (FIG. 20E).

This dual barcoding scheme would eliminate errors due to systematic barcode failure, but introduces the possibility of template switching errors. To explore parameters for this scheme, Applicant assumed that 5% of all sample-specific barcode pairs are not detected due to varying sequencing depth (Δ_{stoch}=0.05; this is independent of dropout due to low viral load) and that template switching occurs 2% of the time (Δ_{switch}=0.02). Under these assumptions, numerical simulations of this dual barcoding scheme (FIGS. 20F-20G) suggests that both the false-negative and false-positive rates of detection will be less than 0.2% as long as the global frequency of positive samples is below 1.6% when using a set of 96 FIP barcodes (m₁=96), 96 BIP barcodes (m₂=96), with 3 of each barcode per patient sample (k₁, k₂=3), requiring 6 out of 9 barcode pairs to be detected for a positive sample (k₁₂′=6), and 10 pools per run (m₃=10). Increasing the number of barcoded FIP and BIP primers to 192 or 384 each lowers the error rates and allows for higher global frequencies of positive samples. All simulated compressed barcoding schemes have not been experimentally validated yet.

### Materials and Methods

### LAMP-Seq

1. A freshly inoculated cotton dry swab (nerbe plus GmbH, 09-819-5000) is inserted into 500 of QuickExtract (Lucigen) supplemented with 2 ng/μl RNase-free plasmid DNA (pX330, Addgene #42230) in a 15 ml Falcon tube, stored on ice for transport, incubated for at least 10 minutes at room temperature, and heated to 95° C. for 5 minutes.
2. A barcoded RT-LAMP reaction is performed, containing the following components:
   a. 100 μl 2×LAMP master mix (NEB, E1700L),
   b. 60 μl 1 M Tris-HCl pH 8.6,
   c. 2 μl RNase-free plasmid DNA (pX330, Addgene #42230, 100 ng/μ1),
   d. 20 μl swab lysate from step 1,
   e. 5 μl Bst 3.0 (NEB, M0374L, 8,000 units/nil),
   f.

1.6 μM total of a unique set of one to five
barcoded C-FIP primers
(TGCGGCCAATGTTTGTAATCAGNNNNNNNNNNCCAAGGAAATTTTGG
GGAC (SEQ ID NO: 173)),

where Ns denote a specific barcode sequence,
g.

1.6 μM C-BIP primer
(CGCATTGGCATGGAAGTCACTTTGATGGCACCTGTGTAG
(SEQ ID NO: 174)),

h.

0.2 μM C-F3 primer
(AACACAAGCTTTCGGCAG (SEQ ID NO: 175)),

i.

0.2 μM C-B3 primer
(GAAATTTGGATCTTTGTCATCC (SEQ ID NO: 176)),

j.

0.4 μM C-LF primer
(TTCCTTGTCTGATTAGTTC (SEQ ID NO: 177)),

k.

0.4 μM C-LB primer
(ACCTTCGGGAACGTGGTT (SEQ ID NO: 178)),

l. water to a total volume of 200 μl.
3. The RT-LAMP reaction is heated to 65° C. for 1 hour, and to 95° C. for 10 minutes.
4. Up to 100,000 reactions are pooled in batches of 1,000 to 10,000 samples per batch.
5. The pool is diluted 1:100,000 in water.
6. For each pool, a 20-cycle 50 μl PCR reaction is performed:
   a. 25 μl NEBNext 2× Master Mix (NEB),
   b.

0.5 μM PCR-C-fwd primer
(ACACTCTTTCCCTACACGACGCTCTTCCGATCTAACGCTGAAGCGCTG
GGGGCAAA (SEQ ID NO: 179)),

c.

0.5 μM PCR-C-rev primer
(TGACTGGAGTTCAGACGTGTGCTCTTCCGATCTGTTTGTAATCAGTTC
CTTGTCTG (SEQ ID NO: 180)),

d. 5 μl of diluted RT-LAMP reactions from step 5,
e. water.
7. For each pool, a secondary 12-cycle 50 μl PCR reaction is performed with:
   a. 25 μl NEBNext 2× Master Mix (NEB),
   b.

0.5 μM pool-specific fwd barcoding primer
(AATGATACGGCGACCACCGAGATCTACACNNNNNNNNNNACACTCTT
TCCCTACACGACGCT (SEQ ID NO: 181)),

where Ns denote a specific barcode sequence,
c.

0.5 μM pool-specific rev barcoding primer
(CAAGCAGAAGACGGCATACGAGATNNNNNNNNNNGTGACTGGAGTT
CAGACGTGTGCT (SEQ ID NO: 182)),

where Ns denote a specific barcode sequence,
d. 5 μl of previous PCR reaction,
e. water.
8. The PCR products are pooled on ice, purified using a silica spin column (Qiagen), quantified using a NanoDrop photospectrometer (Thermo), and sequenced on an Illumina NextSeq sequencer or similar device (A MiSeq sequencer can be used for testing the method, or when screening smaller numbers of samples).
9. Using the LAMP-Seq-Inspector software (http://manuscript.lamp-seq.org/Inspector.htm), barcodes co-occurring with the correct viral genome sequence excluding sequence portions covered by primers are determined.
10. Positive samples are determined using a database of barcode combinations assigned to sample IDs, requiring either one (single barcoding scenario) or at least three out of five sample barcodes (compressed barcode space) being positive.

### DISCUSSION

LAMP and RT-LAMP have been previously established for use as highly sensitive methods for pathogen detection from unpurified human samples with detection limits below 100 nucleic acid molecules. While colorimetric or turbidimetric (Mori and Notomi, 2009) readouts of LAMP reactions can suffer from false positive results (Estrela et al., 2019), this Example at least demonstrates that a sequencing-based readout detects correct fusions of barcode sequences with two stretches of viral sequence. To further increase specificity, viral sequences can be filtered for sequence portions that are non-overlapping with primer sequences. In addition, at least this Example shows that this multiplexing-LAMP strategy can be made robust against barcode cross-contamination originating from template switching events or primer contamination at the PCR stage, as two template switching events would be required in order to create a sequencing-compatible amplicon.

A barcoded RT-LAMP protocol (LAMP-Seq) was developed, optimized, and successfully validated it on 28 human swab samples. The current protocol does not require RNA purification or individual processing steps exceeding one liquid transfer per sample, which can be automated by using matrix-format tubes during swab lysis. Of note, larger numbers of patient samples need to be tested before proposing deployment for diagnostic or population screening purposes. Larger sample sizes will also allow to explore the possibility of rare inhibitory compounds in unpurified human samples, potentially resulting from food intake, hygiene interventions, or super-infections. Apart from further validation studies, compatibility of the current LAMP-Seq protocol with other types of human samples (saliva, sputum, mid-nasal swabs, fecal samples) should be explored rapidly to identify the most scalable solution for unsupervised home testing, as long as guaranteeing safety of postal workers during shipping of the inactivated samples can be addressed. For deployment, LAMP-Seq also has to be equipped with a positive control amplicon to ensure efficient RT-LAMP processing of each individual sample.

A major advantage of the method described and demonstrated herein is that barcoding is performed early in the protocol using a simple heating device like an oven, whereas downstream processing of sequencing libraries is done on large pools of samples. To enable that feature of LAMP-Seq, Applicant demonstrated that multiple barcode sequences can be inserted into the forward inner primer (FIP) and/or backward inner primer (BIP) primer used during an RT-LAMP reaction, as long as a simple sequence motif is avoided in all barcode sequences. Since all barcodes have to be experimentally validated for diagnostic use, a dual-indexing scheme that would allow to uniquely barcode more than 100,000 samples per run while only requiring 192 validated barcoded FIP/BIP primers was described and mathematically simulated.

A potential limitation of the presented approach is that skewing of sample representation at the pooling stage may affect testing sensitivity. Although the LAMP reaction saturates in positive samples largely independent of template concentrations (FIGS. 1E, and 19B), thus equalizing the representation across positive samples in an advantageous manner, the reaction might also add random skewing to pooled samples when scaling to hundreds of thousands of samples; however, preliminary modeling suggests that pooling 100,000 samples per NextSeq run offers robust detection.

LAMP-Seq requires low amounts of consumables with the exception of three proprietary enzymes (<20 USD per sample), which, however, could be mass-produced in E. coli, titrated down in concentration, or be replaced by more cost-effective alternatives. Of note, the established LAMP-Seq protocol used cotton-wood-swabs that are globally available in mass quantities for <5 ct. each. The synthesis cost of the barcode primer library is negligible overall (5,000 USD total for 960 barcodes), leaving point-of-test infrastructure, logistics, and robotics as putative cost driving items. Once successfully established, however, this infrastructure represents a positive externality of the current pandemic, ready to rapidly counter future waves of viral spread or pandemic outbreaks. In this scenario, LAMP-Seq would uniquely allow multiplexing multiple targets to enable scalable differential diagnostics.

### Code Availability

The LAMP-Seq Inspector tool for processing raw LAMP-Seq data is available at: manuscript.lamp-seq.org/Inspector.htm, which is incorporated herein by reference. Python scripts for designing the error-correcting barcodes are available at: github.com/feldman4/dna-barcodes, which is incorporated herein by reference. Jupyter Notebooks for numerical simulations and MATLAB scripts for figure generation are available at: github.com/dbli2000/SARS-CoV2-Bloom-Filter, which is incorporated herein by reference.

### REFERENCES FOR THIS EXAMPLE

Broughton, J. P., Deng, X., Yu, G., Fasching, C. L., Singh, J., Streithorst, J., Granados, A., Sotomayor-Gonzalez, A., Zorn, K., Gopez, A., et al. (2020). Rapid Detection of 2019 Novel Coronavirus SARS-CoV-2 Using a CRISPR-based DETECTR Lateral Flow Assay. 1-27.
Chinazzi, M., Davis, J. T., Ajelli, M., Gioannini, C., Litvinova, M., Merler, S., Pastore y Piontti, A., Mu, K., Rossi, L., Sun, K., et al. (2020). The effect of travel restrictions on the spread of the 2019 novel coronavirus (COVID-19) outbreak. Science eaba9757-12.
Dong, E., Du, H., and Gardner, L. (2020). An interactive web-based dashboard to track COVID-19 in real time. Lancet Infect Dis.
Estrela, P. F. N., de Melo Mendes, G., de Oliveira, K. G., Bailão, A. M., de Almeida Soares, C. M., Assunção, N. A., and Duarte, G. R. M. (2019). Ten-minute direct detection of Zika virus in serum samples by RT-LAMP. Journal of Virological Methods 271, 113675.
Lamb, L. E., Bartolone, S. N., Ward, E., and Chancellor, M. B. (2020). Rapid Detection of Novel Coronavirus (COVID-19) by Reverse Transcription-Loop-Mediated Isothermal Amplification. 1-17.
Li, R., Pei, S., Chen, B., Song, Y., Zhang, T., Yang, W., and Shaman, J. (2020). Substantial undocumented infection facilitates the rapid dissemination of novel coronavirus (SARS-CoV2). Science 6, eabb3221-eabb3229.
Mori, Y., and Notomi, T. (2009). Loop-mediated isothermal amplification (LAMP): a rapid, accurate, and cost-effective diagnostic method for infectious diseases. Journal of Infection and Chemotherapy 15, 62-69.
Nagamine, K., Hase, T., and Notomi, T. (2002). Accelerated reaction by loop-mediated isothermal amplification using loop primers. Molecular and Cellular Probes 16, 223-229.
Notomi, T., Okayama, H., Masubuchi, H., Yonekawa, T., Watanabe, K., Amino, N., and Hase, T. (2000). Loop-mediated isothermal amplification of DNA. Nucl. Acids Res. 28, E63-E63.
Yang, W., Dang, X., Wang, Q., Xu, M., Zhao, Q., Zhou, Y., Zhao, H., Wang, L., Xu, Y., Wang, J., et al. (2020). Rapid Detection of SARS-CoV-2 Using Reverse transcription RT-LAMP method. 1-25.
Zhang, Y., Odiwuor, N., Xiong, J., Sun, L., Nyaruaba, R. O., Wei, H., and Tanner, N. A. (2020). Rapid Molecular Detection of SARS-CoV-2 (COVID-19) Virus RNA Using Colorimetric LAMP. 1-14.

### Example 6. RNA Preparation for COIV-19 Detection

One of the major bottlenecks for COVID-19 diagnosis is the limited availability of RNA extraction kits for preparing virus RNA from patient samples and the low-throughput nature of the extraction procedure. This Example at least describes and demonstrates a one-step extraction-free RNA preparation method that can be carried out in 5 minutes and the reaction can be used directly with the CDC COVID-19 RT-qPCR testing protocol, thus increasing throughput, and alleviating supply chain issues.

### Materials and Reagents

Quick Extract™ DNA Extraction Solution (QE09050), Lucigen

### Protocol

### Step 1.

Dilute nasopharyngeal swab stored in Viral Transport Medium or Human Specimen Control (HSC) 1:1 with Quick Extract™ DNA Extraction Solution. For example, in a fresh PCR tube, mix 20 ul of swab sample with 20 ul of Quick Extract.

### Step 2.

Incubate swab-Quick Extract mix at 95° C. for 5 minutes. Allow reaction to cool on ice before proceeding.

### Step 3.

Use reaction from step (2) for qRT-PCR. Make sure the amount from step (2) does not exceed 10% of the total qRT-PCR reaction volume. For example, if a RT-qPCR reaction has a total volume of 50 ul, do not use more than 5 ul of the reaction mix from step (2).

### Assay Development and Preliminary Validation

Applicants evaluated a number of buffer compositions to identify one that achieved efficient lysis of enveloped virus while preserving the activity of the CDC recommended RT-qPCR reaction (TaqPath™ 1-Step RT-qPCR Master Mix). Of all of the buffers tested, Quick Extract™ DNA Extraction Solution provided satisfactory results.

To confirm that the presence of QE does not interfere with RT-qPCR activity, comparison of RT-qPCR reactions using synthetic SARS-CoV-2 gene fragment (Twist Synthetic SARS-CoV-2 RNA Control 1, SKU: 102019) dissolved in either ddH₂O or in a 50:50 ddH₂O:Quick Extract mixture was performed. Each RT-qPCR reaction was set up with a total volume of 10 ul (1 ul of RNA sample, 0.5 ul of CDC probe N1, 2.5 ul of TaqPath RT-qPCR master mix, and 6 ul of ddH₂O). From these reactions, Applicants found that Quick Extract at a final concentration of 5% did not negatively affect the RT-qPCR reaction (FIG. 1A).

Preliminary validation of the Quick Extract RNA preparation procedure was conducted on coronavirus positive nasopharyngeal swabs and found that RNA samples prepared using Quick Extract supported similarly sensitive detection of coronavirus as QIAmp Viral RNA Miniprep for all 4 swab samples. To simulate low viral load, coronavirus positive swabs were diluted 1:10 in pooled nasopharyngeal swabs from 5 unique, healthy donors (n) prior to purification or Quick Extract treatment. For the QIAmp Viral RNA Miniprep conditions, 100 ul of diluted swab sample was used for extraction and was eluted using 100 ul of ddH₂O. 1 ul of the elution was used in a 10 ul RT-qPCR reaction. For the Quick Extract conditions, 1 ul of Quick Extract preparation was used for each 10 ul RT-qPCR reaction.

### Example 7—Population-Scale COVID-19 Diagnostics Using Combinatorial Barcoding

The ongoing SARS-CoV-2 pandemic has caused devastating losses. Exponential spread can be slowed by social distancing and population-wide isolation measures, but those place a burden on society, and once lifted, exponential spread can re-emerge. Regular population-scale testing, combined with contact tracing and case isolation, should break the cycle of transmission. However, current detection strategies are not capable of such large-scale processing. This Example presents exemplary protocol for LAMP-Seq, a barcoded Revers-Transcription Loop-mediated Isothermal Amplification (RT-LAMP) method that is highly scalable. Relying on world-wide infrastructure for next-generation sequencing, and in the context of population-wide sample collection, LAMP-Seq can be scaled to analyze millions of samples per day.

Currently, most testing for active SARS-CoV-2 infection is performed using viral RNA extraction followed by RT-qPCR to amplify and detect several highly conserved regions of the SARS-CoV-2 genome. The global capacity for testing using this approach, however, has been limited in several ways. Initially, access and supply of reagents and instruments were limited considering the surge in demand. Second, this protocol requires a number of hands-on steps that must be performed by trained professionals, hampering its scalability, although automated systems do increase scale. Third, while several sequencing-based PCR approaches have been proposed (https://docs.google.com/document/d/1kP2w_uTMSep2UxTCOnUhh1TMCjWvHEY0sUUp kJHPYV4, https://www.notion.so/Octant-SwabSeq-Testing-9eb80e793 d7e46348038aa80a5a901fd-639fd74b2ff14daf9a3b78bac1c738b1), their throughput is constrained by the availability of required devices like thermocyclers. Finally, and most critically, even as some of these bottlenecks have been reduced by automation and better supply chains, massive, repeated population testing is hampered by the need to collect samples in centralized settings, and to process each of them individually.

This Example at least describes and demonstrates exemplary protocols of LAMP-Seq, a protocol that allows for population-scale testing using massively parallel RT-LAMP (Nagamine et al., 2002; Notomi et al., 2000) by employing sample-specific barcodes. This approach requires only three heating steps for each individual sample, followed by pooled processing, parallelized deep sequencing, and well-established computational analysis. By using a simple thermal protocol for processing individual samples and pooling many samples prior to resource-intensive steps, the requirement for specialized reagents, equipment, and labor is greatly reduced relative to alternative protocols. Unique tracking of hundreds of millions of samples as well as asynchronous testing logistics, including at-home collection, can be achieved by employing a compressed barcode space. The design of LAMP-Seq, validation on clinical specimens, and simulated barcoding strategies are described. It is estimated that the cost per sample would be less than 20 USD based on current list-prices of off-the-shelf products (excluding labor and instrument costs), with a potential for at least 10-fold cost reduction through scaled sourcing of three enzymes (RTx, Bst 2.0, Bst 3.0). Most importantly, this approach is predicted to be scalable to hundreds of thousands of samples per day per sequencing facility and could be deployed in developing countries.

### Results

LAMP-Seq is an approach for population-scale testing for SARS-CoV-2 infection with the following overall steps (FIGS. 1A-1C): a barcoded RT-LAMP reaction is performed on an unpurified or lysed swab sample with primers specific for the SARS-CoV-2 genome, which is followed by large-scale pooling of samples, PCR amplification with additional barcoding, deep sequencing, and data analysis to identify positive individuals (FIGS. 1A-1B): see below for detailed protocol). RT-LAMP reactions have been demonstrated to be highly sensitive for sequence-specific viral nucleic acid detection (Lamb et al., 2020; Yang et al., 2020; Zhang et al., 2020), even from unpurified samples (Estrela et al., 2019). To establish a barcoded RT-LAMP reaction, barcode sequences were inserted into the forward inner primer (FIP), which enables generation of barcoded palindromic amplification products (FIG. 1C) When a small fraction of samples is expected to be positive during population scale testing, the number of unique barcode primers needed for testing a large number of samples can be further limited by using a compressed barcode space (below).

Specifically, three barcoded primer sets were designed based on validated RT-LAMP amplicons (See Table 51 of Schmid-Burgk et al. 2020. BioRxiv https://doi.org/10.1101/2020.04.06.025635, which is incorporated by reference as if expressed in its entirety herein), (Broughton et al., 2020; Lamb et al., 2020; Zhang et al., 2020)) perfectly matching 95.0% (amplicon A), 95.4% (amplicon B), and 96.8% (amplicon C) of 4,406 SARS-CoV-2 genomes available in the NCBI database (May 30, 2020). 10-nt barcodes with GC content of 30%-70% and lacking homopolymer repeats of four or more nucleotides were inserted into the FIP primer. It was ensured that barcodes are robust to sequencing errors by a minimum Levenshtein edit distance between any barcode pair sufficient to detect two insertion, deletion or substitution errors (See Table 51 of Schmid-Burgk et al. 2020. BioRxiv https://doi.org/10.1101/2020.04.06.025635, which is incorporated by reference as if expressed in its entirety herein).

Comparing barcoded LAMP reactions to non-barcoded controls using a dsDNA surrogate template for SARS-CoV-2, the presence of a prototypical 10-nt barcode within the FIP primer did not affect LAMP sensitivity, product amounts, or downstream PCR amplification was confirmed (FIG. 1D). Templating two individually barcoded LAMP reactions that differ 100-fold in the amount of dsDNA template, combining them for PCR amplification, and sequencing the products resulted in read numbers within a two-fold range between the two samples (FIG. 1E), indicating that RT-LAMP saturation can effectively compress the dynamic range of input viral loads. This can be beneficial when analyzing many samples together on a sequencing run. Furthermore, the expected sequence of barcoded RT-LAMP-PCR products was confirmed by Illumina sequencing (FIG. 1F). In order to determine the molecular sensitivity of barcoded RT-LAMP reactions, 24 reactions with differing numbers of template RNA molecules was performed and it was determined positive subsequent PCR reactions by gel electrophoresis (FIG. 1G). Using a constant per-molecule probability model of RNA detection, the maximum likelihood estimate for molecular detection efficiency is 1/35.5 per RNA molecule (FIG. 111) which corresponds to an LoD-95 of 105 molecules. This is about an order of magnitude less sensitive than RT-qPCR (https://www.fda.gov/media/134922/download).

To validate LAMP-Seq, 28 human samples were tested side-by-side with a standard clinical diagnostic by RT-qPCR with a human subjects protocol approved by the ethics committee of the University Hospital Bonn. Two oropharyngeal samples were collected from each individual using two separate cotton swabs, which were anonymized using an individual ID. One swab was analyzed using a standard clinical diagnostics pipeline comprising rehydration, robotic RNA purification, and RT-qPCR (FIG. 19A, upper panel). The other swab was immediately inserted into a tube containing QuickExtract lysis buffer (Joung et al., 2020) (FIG. 19A, lower panel), processed and sequenced according to the LAMP-Seq protocol using individual PCR barcodes (Methods). The two methods were in complete agreement on both positives and negatives (FIGS. 19B-19C): All 12 individuals identified as SARS-CoV-2 RNA positive by RT-qPCR were also detected positive using LAMP-Seq employing a threshold of 10,000 reads; the remaining 16 individuals were identified as negative for viral RNA in agreement between both methods (FIGS. 19B-19C), with an average of 962 LAMP-Seq reads per negative sample, putatively arising from barcode swapping. Of note, N-gene-specific primers have been reported to be slightly less sensitive in RT-qPCR than primers for other targets (Corman et al., 2020). Unfiltered LAMP-Seq sequencing data confirmed the expected read structure, comprising primer sequences, viral genome sequence, and a matching barcode in 67% of reads (FIG. 19D), while the majority of remainder reads bore single-nucleotide substitutions or truncations relative to the expected amplicon sequence.

Effective SARS-CoV-2 virus inactivation in QuickExtract lysis buffer was confirmed both after 30 minutes of incubation at 65° C. and after 10 minutes at 95° C., resulting in at least a 3.9E4-fold reduction in viral infectivity (Table 7), whereas residual SARS-CoV-2 infectivity was retained following 30 minutes incubation at 22° C. To further investigate the inactivation efficiency of the lysis buffer, a high dose of VSV virus was used to demonstrate at least a 1E7-fold reduction in viral infectivity (Table 7),

TABLE 7

| | | Viral Inactivation | | | | |
|---|---|---|---|---|---|---|
| Incubation | | | | Residual | | |
| Parameters | | | Viral | viable | Limit of | Validated |
| Temper- | Time | | Inoculation | virus | Detection | log 10 |
| ature | (minutes) | Virus | (log 10) | (CPE) | (log10) | reduction |
| 65° C. | 20 | VSV | 9 | No | 2 | >7 |
| 22° C. | 30 | SARS- | 6 | Yes | 2 | |
| 65° C. | 30 | CoV-2 | 6 | No | 2 | >4.6 |
| 95° C. | 10 | | 6 | no | 2 | >4.6 |

Next, LAMP-Seq was optimized to allow successful pooling of barcoded RT-LAMP reactions, which is essential for scaling up LAMP-Seq, focusing on minimizing levels of barcode swapping, and on ensuring a sufficient number of individually validated barcodes. When six barcoded RT-LAMP reactions were pooled, of which three were templated with RNA, and performed PCR and sequencing, moderate levels of barcode swapping was observed (FIG. 19E, left panel). It was hypothesized that barcode primers being transferred into the PCR reaction may lead to amplification and re-barcoding of amplicons. Detectable barcode swapping was eliminated by diluting pooled RT-LAMP reactions one-million-fold in the PCR reaction, which (FIG. 19E, right panel). Next, pooling 480 barcoded FIP primers, performing RT-LAMP reactions in four replicates, and sequencing the barcode distribution in resulting products revealed that ˜5% of barcode sequences perform poorly or even fail to engage in LAMP-Seq (FIG. 19F). Investigating potential sequence determinants that could guide optimized barcode design, it was observed that the least efficient barcode primers displayed a marked enrichment for a GTCC motif or truncations thereof, especially towards the 3′ end of the barcode (FIG. 19F, inset). As this motif is the reverse complement of the 3′ end of the FIP primer, we hypothesize it could sequester the 3′ end by forming an intramolecular structure, thus inhibiting elongation of the primer, and should be avoided. Following this rule, >6,000 barcoded FIP and BIP primers were designed 240 validated barcoded FIP primers for application of LAMP-Seq were provided (See Table 51 of Schmid-Burgk et al. 2020. BioRxiv https://doi.org/10.1101/2020.04.06.025635, which is incorporated by reference as if expressed in its entirety herein).

A high-output Illumina 75-cycle NextSeq run can routinely generate 200 million sequencing reads in 14 hours, which we predict is sufficient for 100,000 samples per run, even accounting for library skewing due to differences in viral loads (for modeling see Examples 3 and 8 herein). Barcoding 100,000 samples could be achieved by a naïve approach, where each sample is contacted with a unique barcode primer (FIG. 20A, left). However, as synthesis, validation, and robotic handling of large numbers of barcode primers is challenging, we explored a compressed barcode space, where every sample would be assigned a unique combination of more than one barcode (FIG. 20A, right). For this scenario, we conservatively assume that 1% of synthesized barcode primers systematically fail to work, even after removing barcode primers that contain homology to GTCC (Δ_{synth}=0.01), and that 5% of all sample-specific barcodes are not detected due to varying sequencing depth (Δ_{stoch}=0.05; independent of dropout due to low viral load). For automated assembly of testing reactions with unique barcode combinations, we anticipate that m=1,000 barcode primers can be easily handled by available pipetting robots. Under these assumptions, we investigated for 100,000 samples what number of barcodes per sample (k), total number of barcode primers (m), and number of pools per run (m₂) would minimize false-positive and false-negative rates of detection (FIGS. 20B-20C). Interpreting the compressed barcoding problem as a modified Bloom filter (see e.g., Example 3 herein), we predict that when using k=5 barcodes per sample, where k′=3 barcodes are detected per sample, and splitting samples into m₂=96 pools per run, both the false-negative and false-positive rates of detection using a compressed barcode space will be less than 0.2% as long as the global frequency of positive samples is below 1.2% (FIG. 20B). Larger numbers of barcodes will further lower error rates and ensure performance in the face of higher global positive frequencies (FIG. 20C).

As some barcoded FIP primers fail in the RT-LAMP reaction, it can be advantageous to reduce the number of barcode primers that need to be validated. One way to achieve this is with a dual barcoding scheme, where both the FIP and BIP primers are barcoded (FIG. 20D). Using Tn5-mediated tagmentation and sequencing of RT-LAMP products (Thi et al., 2020), the formation of RT-LAMP products with the FIP/BIP barcode insertion sites facing each other was experimentally quantified to occur with a frequency of 33.1%, which suffices for PCR amplification of barcode pairs (FIG. 20D, red numbers). Without compression, 100,000 patient samples could be uniquely barcoded using 100 FIP primers, 100 BIP primers, and 10 pools per run. With compression, over 20 billion samples can each be assigned a unique combination of barcodes using a combination of 3 FIP primers and a combination of 3 BIP primers per patient sample from a pool of 96 barcoded FIP and 96 barcoded BIP primers (FIG. 20E).

This dual barcoding scheme would eliminate errors due to systematic barcode failure, but introduces the possibility of template switching errors. To explore parameters for this scheme, we assume that 5% of all sample-specific barcode pairs are not detected due to varying sequencing depth (Aston=0.05; this is independent of dropout due to low viral load) and that template switching occurs 2% of the time (Δ_{switch}=0.02). Under these assumptions, numerical simulations of this dual barcoding scheme (FIGS. 20F-20G, Example 8) suggests that both the false-negative and false-positive rates of detection will be less than 0.2% as long as the global frequency of positive samples is below 1.6% when using a set of 96 FIP barcodes (m₁, =96), 96 BIP barcodes (m₂=96), with 3 of each barcode per patient sample (k₁, k₂=3), requiring 6 out of 9 barcode pairs to be detected for a positive sample (k₁₂′=6), and 10 pools per run (m₃=10) (FIG. 20G). Increasing the number of barcoded FIP and BIP primers to 192 or 384 each and changing the required threshold to 5 out of 9 barcode pairs detected for a positive sample (k₁₂′=5) lowers the error rates and allows for higher global frequencies of positive samples (FIG. 20F). It is emphasized that the simulated compressed barcoding schemes have not been experimentally validated yet.

### DISCUSSION

LAMP and RT-LAMP (Nagamine et al., 2002; Notomi et al., 2000) have been previously established for use as highly sensitive methods for pathogen detection from unpurified human samples with detection limits below X100 nucleic acid molecules (Mori and Notomi, 2009). Although colorimetric or turbidimetric readouts of LAMP reactions can suffer from false positive results (Estrela et al., 2019), here it is demonstrated that a sequencing-based readout provides maximum specificity by detecting only correct fusions of barcode sequences with two stretches of viral sequence. In addition, it is shown that this novel multiplexing-LAMP strategy can be made robust against barcode cross-contamination originating from template switching events or primer contamination at the PCR stage, as two template switching events would be required in order to create a sequencing-compatible amplicon.

A barcoded RT-LAMP protocol (LAMP-Seq) was developed, optimized, and successfully validated on 28 human swab samples. The current protocol does not require RNA purification or individual processing steps except using approximately one pipette tip per sample, which can be automated through using matrix-format tubes at the stage of swab lysis. Of note, larger numbers of patient samples need to be tested before proposing deployment of LAMP-Seq for population screening. Larger sample sizes will also allow exploration of the possibility of rare inhibitory compounds in some unpurified human samples, potentially resulting from food intake, hygiene interventions, or the oral microbiome. Apart from further validation studies, compatibility of the current LAMP-Seq protocol with other types of human samples (saliva, sputum, anterior nasal (AN) swabs, mid-nasal swabs, fecal samples) should be explored rapidly to identify the most scalable solution for unsupervised at-home sample collection, which would be attractive if the safety can be guaranteed during shipping of inactivated samples. For deployment, LAMP-Seq also has to be equipped with a positive control amplicon to ensure efficient RT-LAMP processing of each individual sample, which could run in the same RT-LAMP reaction or in a separate reaction, allowing independent saturation of both amplicons. Of note, the compressed barcoding schemes would require the positive control template to bear an additional heterogeneous sequence portion.

A major advantage of LAMP-Seq is that barcoding is performed early in the protocol using a simple heating device (like an oven), whereas downstream processing of sequencing libraries is done on large pools of samples. To enable pooled processing, it was shown that multiple barcode sequences can be inserted into the forward inner primer (FIP) and/or backward inner primer (BIP) primer used during an RT-LAMP reaction, as long as a simple sequence motif is avoided in all barcode sequences. As all barcodes have to be experimentally validated for diagnostic use, we propose and mathematically simulate a dual-indexing scheme that would allow uniquely barcoding more than 100,000 samples per run while only requiring 192 validated barcoded FIP/BIP primers.

A potential limitation of the presented approach is that skewing of sample representation at the pooling stage may affect testing sensitivity. Although the LAMP reaction saturates in positive samples largely independent of template concentrations (FIG. 1E), thus equalizing the representation across positive samples in an advantageous manner, the reaction might also add random skewing to pooled samples when scaling to hundreds of thousands of samples; however, preliminary modeling suggests that pooling 100,000 samples per NextSeq run offers robust detection (Examples 3 and 8).

LAMP-Seq requires low amounts of consumables with the exception of three proprietary enzymes and buffer compositions; however, these enzymes could be mass-produced using E. coli or replaced by open-source alternatives. The established LAMP-Seq protocol used cotton-wood swabs that are available in mass quantities for <5 ct. each. The synthesis cost of the barcode primer library is low overall (5,000 USD total for 960 barcodes, <10 ct. per sample), leaving point-of-test infrastructure, logistics, and robotics as putative cost driving items. Once successfully established, however, this infrastructure could rapidly counter future waves of viral spread or pandemic outbreaks. Of note, LAMP-Seq could uniquely allow multiplexing multiple targets (of different viruses) to enable scalable differential diagnostics.

Broadly similar approaches of barcoded isothermal amplification methods have been independently suggested by other researchers (https://hms.harvard.edu/news/soup-nuts; Thi et al.; 2020; Palmieri et al.; Wu et al., 2020). To facilitate open communication, a forum on www.LAMP-Seq.org has been set up.

### LAMP-Seq Testing for SARS

1. A freshly inoculated cotton dry swab (nerbe plus GmbH, 09-819-5000) is inserted into 500 μl of QuickExtract (Lucigen) supplemented with 2 ng/μ1 RNase-free plasmid DNA (pX330, Addgene #42230) in a 15 ml Falcon tube, stored on ice for transport, incubated for at least 10 minutes at room temperature, and heated to 95° C. for 5 minutes.
2. A barcoded RT-LAMP reaction is performed, containing the following components:
   a. 100 μl 2×LAMP master mix (NEB, E1700L),
   b. 60 μl 1 M Tris-HCl pH 8.6,
   c. 2 μl RNase-free plasmid DNA (pX330, Addgene #42230, 100 ng/μ1),
   d. 20 μl swab lysate from step 1,
   e. 5 μl Bst 3.0 (NEB, M0374L, 8,000 units/ml),
   f.

1.6 μM total of a unique set of one to five barcoded C-FIP primers
(TGCGGCCAATGTTTGTAATCAGNNNNNNNNNNCCAAGGAAATTTTGG
GGAC (SEQ ID NO: 183)),

where Ns denote a specific barcode sequence,
g.

1.6 μM C-BIP primer
(CGCATTGGCATGGAAGTCACTTTGATGGCACCTGTGTAG
(SEQ ID NO: 184)),

h.

0.2 μM C-F3 primer
(AACACAAGCTTTCGGCAG (SEQ ID NO: 185)),

i.

0.2 μM C-B3 primer
(GAAATTTGGATCTTTGTCATCC (SEQ ID NO: 186)),

j.

0.4 μM C-LF primer
(TTCCTTGTCTGATTAGTTC (SEQ ID NO: 187)),

k.

0.4 μM C-LB primer
(ACCTTCGGGAACGTGGTT (SEQ ID NO: 188)),

l. water to a total volume of 200 μl.
3. Optionally, the RT-LAMP reaction is split into eight reactions.
4. The RT-LAMP reaction is heated to 65° C. for 1 hour, and to 95° C. for 10 minutes.
5. Up to 100,000 reactions are pooled in batches of 1,000 to 10,000 samples per batch.
6. The pool is diluted 1:100,000 in water.
7. For each pool, a 20-cycle 50 μl PCR reaction is performed:
   a. 25 μl NEBNext 2× Master Mix (NEB),
   b.

0.5 μM PCR-C-fwd primer
(ACACTCTTTCCCTACACGACGCTCTTCCGATCTAACGCTGAAGCGCTG
GGGGCAAA (SEQ ID NO: 189)),

c.

0.5 μM PCR-C-rev primer
(TGACTGGAGTTCAGACGTGTGCTCTTCCGATCTGTTTGTAATCAGTTC
CTTGTCTG (SEQ ID NO: 190)),

d. 5 μl of diluted RT-LAMP reactions from step 5,
e. water.
8. For each pool, a secondary 12-cycle 50 μl PCR reaction is performed with:
   a. 25 μl NEBNext 2× Master Mix (NEB),
   b.

0.5 μM pool-specific fwd barcoding primer
(AATGATACGGCGACCACCGAGATCTACACNNNNNNNNNNACACTCTT
TCCCTACACGACGCT (SEQ ID NO: 191)),

where Ns denote a specific barcode sequence,
c.

0.5 μM pool-specific rev barcoding primer
(CAAGCAGAAGACGGCATACGAGATNNNNNNNNNNGTGACTGGAGTT
CAGACGTGTGCT (SEQ ID NO: 192)),

where Ns denote a specific barcode sequence,
d. 5 μl of previous PCR reaction,
e. water.
9. The PCR products are pooled on ice, purified using a silica spin column (Qiagen), quantified using a NanoDrop photospectrometer (Thermo) or Qubit (Thermo), and sequenced on an Illumina NextSeq sequencer or similar device (A MiSeq sequencer can be used for testing the method, or when screening smaller numbers of samples).
10. Using the LAMP-Seq-Inspector software (http://manuscript.lamp-seq.org/Inspector.htm), barcodes co-occurring with the correct viral genome sequence excluding sequence portions covered by primers are determined. This analysis can also be performed using a “kallisto bustools” workflow (Booeshaghi et al., 2020).
11. Positive samples are determined using a database of barcode combinations assigned to sample IDs, requiring either one (single barcoding scenario) or at least three out of five sample barcodes (compressed barcode space) being positive.

### Clinical RT-qPCR Pipeline

Swabs were rehydrated in 650 μl PBS. Viral RNA was extracted using the chemagic™ Prime Viral DNA/RNA 300 Kit (PerkinElmer) on a Chemagic Prime 8 system (PerkinElmer). 290 μl viral sample were mixed with 10 μl extraction control sample and 300 μl lysis buffer. Extraction was performed according to the manufacturers protocol and viral RNA was eluted in 45 μl elution buffer for subsequent analysis. Detection of viral RNA using one-step real-time reverse-transcription PCR was performed according to (Corman et al., 2020) with iTaq Universal Probes One-Step Kit (Biorad) using primers and probes against the N-gene (N_Sarbeco_F: CACATTGGCACCCGCAATC (SEQ ID NO: 193), N_Sarbeco_R: GAGGAACGAGAAGAGGCTTG (SEQ ID NO: 194), N_Sarbeco_P: FAM-ACTTCCTCAAGGAACAACATTGCCA (SEQ ID NO: 195)-BBQ, TIB MolBiol). Spike-in RNA of the bacteriophage MS2 served as an internal control and was detected with Luna® Universal Probe One-Step RT-qPCR Kit (New England Biolabs) using corresponding primers and probe (MS2_F: TGCTCGCGGATACCCG (SEQ ID NO: 196), Ms2_R: AACTTGCGTTCTCGAGCGAT (SEQ ID NO: 197), MS2_P: YAK-ACCTCGGGTTTCCGTCTTGCTCGT (SEQ ID NO: 198)-BBQ, TIB MolBiol). The reaction for the internal control was performed using dual detection of FAM and YAK/VIC in a Lightcycler 480 (Roche), the detection of the N-gene was done in a QuantStudio5 cycler (Thermo Fisher).

### Viruses and Cells

SARS-CoV-2 strain MUC-IMB-1 was isolated and kindly supplied by Rosina Ehmann and Gerhard Dobler (Bundeswehr institute of microbiology, Munich, Germany). The virus was propagated and titrated on VERO-E6 cells (ATCC CRL-1586). All handling and working with SARS-CoV-2 was conducted in a BSL-3 facility in accordance with the biosafety guidelines of the IIBR. Vesicular stomatitis virus (VSV) serotype Indiana, kindly provided by Eran Bacharach (Tel-Aviv University, Israel), was propagated and titrated on Vero cells (ATCC CCL-81). Handling and working with VSV was conducted in a BSL-2 facility in accordance with the biosafety guidelines of the IIBR.

### Lysis Buffer Inactivation Assay

Quick extract DNA extraction solution (Lucigen) was tested in accordance with the manufacturer's suggested buffer to sample ratio. Universal Transfer Medium (UTM, Copan) aliquots were inoculated with either 5E6 pfu/ml SARS-CoV-2 or 2E9 pfu/ml VSV viruses and were incubated at 22° C., 65° C., or 95° C. for 10 to 30 minutes. Positive and negative control samples included UTM inoculated with viable virus without lysis buffer and UTM with Lysis buffer without virus, respectively. The limit of detection was defined as the first serial dilution of negative control that did not cause CPE by itself (represented in log scale). Briefly, VERO-E6 (for SARS-CoV-2) or VERO cells (for VSV) were cultured in DMEM supplemented with 10% FBS, MEM non-essential amino acids, 2 mM L-Glutamine, 100 U/ml penicillin, 0.1 mg/ml streptomycin, and 12.5 U/ml Nystatin (Biological Industries, Israel). Monolayers (2.5E5 cells per well in 24-well plates) were washed once with MEM Eagles medium without FBS, and infected with 200 μl of ten-fold serial dilutions of the samples. After one hour of incubation the wells were overlaid with 1 ml of MEM medium containing 2% fetal calf serum (FCS), MEM non-essential amino acids, 2 mM L-Glutamine, 100 U/ml penicillin, 0.1 mg/ml streptomycin, 12.5 U/ml Nystatin, and 0.15% Sodium Bicarbonate (Biological Industries, Israel). The cells were then incubated at 37° C., 5% CO₂ for five days (SARS-CoV-2) or one day (VSV). CPE was determined by counter-staining with crystal violet solution.

### Code and Data Availability

The LAMP-Seq Inspector tool for processing raw LAMP-Seq data is available at: http://manuscript.lamp-seq.org/Inspector.htm. Python scripts for designing the error-correcting barcodes are available at: https://github.com/feldman4/dna-barcodes. Jupyter Notebooks for numerical simulations and MATLAB scripts for figure generation are available at: https://github.com/dbli2000/SARS-CoV2-Bloom-Filter. Example LAMP-Seq data is available on www.LAMP-Seq.org.

### Example 8—LAMP-Seq: Population-Scale COVID-19 Diagnostics Using Combinatorial Barcoding—Dual Barcoding

Building on Example 3 herein, an alternative form of barcoding is now considered where patient samples can be barcoded with a combination of barcoded FIP and barcoded BIP primers. Various scenarios were modeled with this dual barcoding scheme, characterize error rates, introduce a template switching error term Δ_{switch}, and simulate realistic parameter sets.

The overall goal here is to design a setup that achieves false negative probabilities (FNPs) and false positive probabilities (FPPs) of <0.2% using a minimal number of total barcodes given parameters of 10 sub-batches per run, 1% positive patient samples, reasonable error rates (Δ_{stoch}=0.05, Δ_{synth}=0, Δ_{switch}=0.02), and either 100,000 or 1,000,000 patients per batch.

There are three barcodes available: patient samples will be individually barcoded at the RT-LAMP stage with a first set of barcodes (barcode 1—FIP, barcode 2—BIP), and then groups of patient samples will be barcoded with an additional set of orthogonal barcodes (barcode 3) in the process of preparing samples for Illumina sequencing. After sequencing, a given combination of the three barcodes can be called as positive or negative for SARS-CoV-2 viral RNA.

Let there be m1 total unique barcode 1s, m2 total unique barcode 2s, and m3 total unique barcode 3s. Each patient sample receives k₁ pre-assigned barcode 1s and k₂ pre-assigned barcode 2s. All other notation remains the same as Example 3. Each barcode 1 or barcode 2 is assumed to fail globally with probability Δsynth, and a specific barcode 1-barcode 2 pair for a patient sample is assumed to fail with probability Δ_{stoch}. There are three scenarios considered below: one where k₁=k₂=1, one where k₁>1 and k₂=1, and one where k₁, k₂>1

Scenario 3: k₁=k₂=1

If b<m₁·m₂·m₃, then every sample in a batch can be assigned a unique barcode. For testing b=100,000 samples, 100 barcode 1s, 100 barcode 2s, and 10 barcode 3s would suffice. Testing b=1,000,000 samples would only require an increase in the number of barcode 3s to 100. Then, every patient sample in a batch would have a unique barcode 1-barcode 2-barcode 3 group.

It is unlikely, although not impossible, for N<m₁·m₂·m₃, which would enable a unique barcode for every sample from the total population. This suggests that batches would have to be defined in some way, and that this scenario is better suited for synchronous testing.

Since each patient has a unique barcode group, there are no false positives. However, with barcode loss, there may be false negatives. Each patient sample now has two barcodes in the RT-LAMP reaction, so the overall false negative probability is 1−(1−Δ_{stoch})(1−Δ_{synth})².

2. Scenario 4: k₁>1, k₂=1

With a liquid handler, it may be possible to give each patient sample k₁ different barcode 1s, with k₁>1, and a single barcode 2 in a pre-assigned way. Then, if

$$N < ( \begin{matrix}{m}_{1} \\ {k}_{1}\end{matrix} ) \cdot {m}_{2} ,$$

every patient sample for the entire population could have a unique combination of barcode 1s and barcode 2s. This does not mean that every barcode 1-barcode 2 pair would correspond to a unique patient sample, as this is only possible if

$$N < \frac{m 1 m 2}{{k}_{1}} .$$

For this scenario, an asynchronous sample collection system is imagined where b of these samples, as they come in, are split into m₃ sub-batches. Each sub-batch then gets an additional unique barcode 3. Patient samples are inferred as positive if, after sequencing, ≥k′₁₂ out of the k₁ corresponding barcode 1-barcode 2-barcode 3 groups are positive.

This is similar to scenario 2 in Supplementary Note 1, except that the barcode 2s here provides a psuedo-sub-batch barcode without requiring physical distribution of samples. We assume that the number of barcode 2s, m₂, is small enough that it is possible to validate each barcode, so we do not consider Δ_{synth} errors for barcode 2s. Combining this with the barcode 3s, we effectively have m₂ m₃ separate non-overlapping sub-pools with b/m2m3 samples each.

Since the barcode 1-barcode 2 pairs no longer correspond to unique patient samples, false positives are possible using this approach. False negatives are also still possible, as a positive sample could be inferred as a negative sample if more than k₁-k′₁₂ corresponding barcode groups are lost. Using a modified Bloom filter model (see Section 8 of this Example (Math Appendix), Example 3, and Burton H. et al. 1970. Space/time trade-offs in has coding with allowable errors. Commun. ACM 13, 7 (July 1970), 422-426. DOI: https://doi.org/10.11445/362686.362692) the false positive probability (FPP_{Δk′}_{₁₂}_{,4}) and false negative probability (FNP_{Δk′}_{₁₂}_{,4}) for this approach can be computed as (Eq. 49)

$$\begin{matrix}{FPP}_{Δ {k}_{12}^{′} , 4} = {\sum }_{i = {k}_{12}^{′}}^{{k}_{1}} ( \begin{matrix}{k}_{1} \\ i\end{matrix} ) {( {Δ}_{synth} + ( 1 - {Δ}_{synth} ) {( 1 - \frac{1 - {Δ}_{stoch}}{{m}_{1}} )}^{\frac{{k}_{2} n}{{m}_{3} {m}_{3}}} )}^{{k}_{1} - i} \\ {( ( 1 - {Δ}_{synth} ) ( 1 - {( 1 - \frac{1 - {Δ}_{stoch}}{{m}_{1}} )}^{\frac{{k}_{1} n}{{m}_{2} {m}_{3}}} ) )}^{i}\end{matrix} \qquad ( 49 )$$

$$\begin{matrix}{FNP}_{Δ {k}_{12}^{′} , 4} = 1 - {\sum }_{i = {k}_{12}^{′}}^{{k}_{1}} ( \begin{matrix}{k}_{1} \\ i\end{matrix} ) {( {Δ}_{synth} + ( 1 - {Δ}_{synth} ) {{Δ}_{stoch} ( 1 - \frac{1 - {Δ}_{stoch}}{{m}_{1}} )}^{{k}_{1} ( \frac{n}{{m}_{2} {m}_{3}} - 1 )} )}^{{k}_{1} - i} \\ {( ( 1 - {Δ}_{synth} ) ( 1 - {{Δ}_{stoch} ( 1 - \frac{1 - {Δ}_{stoch}}{{m}_{1}} )}^{{k}_{1} ( \frac{n}{{m}_{3} {m}_{3}} - 1 )} ) )}^{i}\end{matrix} .$$

These expressions are similar to those in Scenario 2, and analyses there apply here as well.

3. Scenario 5: k₁, k₂>1

It may also be possible to give each patient sample k₁ different barcode 1s and k₂ different barcode 2s, with k₁, k₂>1, in a pre-assigned way. Then, if

$$N < ( \begin{matrix}{m}_{1} \\ {k}_{1}\end{matrix} ) \cdot ( \begin{matrix}{m}_{2} \\ {k}_{2}\end{matrix} ) ,$$

every patient sample for the entire population could have a unique combination of barcode is and barcode 2s. This does not mean that every barcode 1-barcode 2 pair would correspond to a unique patient sample, as this is only possible if

$$N < \frac{{m}_{1} {m}_{2}}{{k}_{1} {k}_{2}} .$$

The RT-LAMP reaction randomly incorporates FIP and BIP primers during amplification, so a positive patient sample can produce all k₁·k₂ barcode pairs. As an example, consider k₁=k₂=3. Then 3 barcoded FIP primers and 3 barcoded BIP primers per RT-LAMP reaction would be introduced with a result of 9 distinct barcode 1-barcode 2 pairs per sample.

A asynchronous sample collection system is imagined for this scenario, similar to scenario 4, and will consider 2 schemes for decoding which patients are positive. In the first decoder scheme, a patient sample is inferred to be positive, if, after sequencing, at least k′₂ of the k₂ patient-specific barcode 2s contain at least k′₁ of the k₁ patient-specific barcode 1-barcode 2 pairs. In the second decoder scheme, a patient sample is inferred to be positive if, after sequencing, ≥k′₁₂ out of the k₁·k₂ patient-specific barcode 1-barcode 2-barcode 3 groups come up as positive.

This is similar to scenario 2 in Example 3, with physical placement in a combi-nation of k₂ wells corresponding to the combination of k₂ primers per sample here. The random barcode selection among samples allows us to calculate approximate error rates. It was assumed that the number of barcode 2s, m₂, is small enough that it is possible to validate each barcode, so we do not model Δ_{synth} errors for barcode 2s.

The FPP and FNP of the first decoder scheme can be approximated using equations from Example 3; they are excluded here. Under the second decoder scheme, the false positive probability (FPP_{Δk′}_{₁₂}_{,5}) and false negative probability (FNP_{Δ′k}_{₁₂}_{,5}) are approximately given by (Eq. 50)

$$\begin{matrix}{FPP}_{Δ {k}_{12}^{′} , 5} = {\sum }_{i = {k}_{12}^{′}}^{{k}_{1} {k}_{2}} ( \begin{matrix}{k}_{1} {k}_{2} \\ i\end{matrix} ) {( {Δ}_{synth} + ( 1 - {Δ}_{synth} ) {( 1 - \frac{1 - {Δ}_{stoch}}{{m}_{1}} )}^{\frac{{k}_{1} {k}_{2} n}{{m}_{2} {m}_{3}}} )}^{{k}_{1} {k}_{2} - i} \\ {( ( 1 - {Δ}_{synth} ) ( 1 - {( 1 - \frac{1 - {Δ}_{stoch}}{{m}_{1}} )}^{\frac{{k}_{1} {k}_{2} n}{{m}_{2} {m}_{3}}} ) )}^{i}\end{matrix} \qquad ( 50 )$$

$$\begin{matrix}{FNP}_{Δ {k}_{12}^{′} , 5} = - {\sum }_{i = {k}_{12}^{′}}^{{k}_{1} {k}_{2}} ( \begin{matrix}{k}_{1} {k}_{2} \\ i\end{matrix} ) {( {Δ}_{synth} + ( 1 - {Δ}_{synth} ) {{Δ}_{stoch} ( 1 - \frac{1 - {Δ}_{stoch}}{{m}_{1}} )}^{{k}_{1} ( \frac{{k}_{2} n}{{m}_{2} {m}_{3}} - 1 )} )}^{{k}_{1} {k}_{2} - i} \\ {( ( 1 - {Δ}_{synth} ) ( 1 - {{Δ}_{stoch} ( 1 - \frac{1 - {Δ}_{stoch}}{{m}_{1}} )}^{{k}_{1} ( \frac{{k}_{2} n}{{m}_{2} {m}_{3}} - 1 )} ) )}^{i} + 1\end{matrix} .$$

As the number of positive samples per batch n increases, the false positive probability increases and the false negative probability decreases. An upper bound on the false negative probability (FPP_{Δk′}_{₁₂}_{,5, max}) can be computed that avoids this dependence as (Eq 51)

$$\begin{matrix}{FNP}_{Δ {k}_{12}^{′} , 5 , max} = - {\sum }_{i = {k}_{12}^{′}}^{{k}_{1} {k}_{2}} ( \begin{matrix}{k}_{1} {k}_{2} \\ i\end{matrix} ) {( {Δ}_{synth} + ( 1 - {Δ}_{synth} ) {Δ}_{stoch} )}^{{k}_{1} {k}_{2} - i} \\ {( ( 1 - {Δ}_{synth} ) ( 1 - {Δ}_{stoch} ) )}^{i} + 1\end{matrix} . \qquad ( 51 )$$

Overall, a lower k₂ for a fixed k₁·k₂ leads to a reduced false negative probability at the cost of an increased false positive probability. The effect of varying k₁ and k₂ depends on the chosen k′₁₂ on the proportion of the population infected p.

### 4. Modeling Template Switching

Under this dual barcoding scheme, template switching errors may occur during the PCR before sequencing. These are errors that lead to barcode swapping/index swapping between barcode 1-barcode 2 pairs. Initial experiments have shown that the residual primers of negative samples do not lead to noticeable amounts of template swapping, likely due to sample dilution, so all template switching is assumed to occur between barcode 1-barcode 2 pairs of positive samples. As an example, consider two positive patient samples: one with barcode 1-barcode 2 pair A-B and one with barcode 1-barcode 2 pair C-D. Template switching would produce A-D and B-D as products. Each template switching product as occurring with probability Δ_{switch} were modeled. This probability has not been well characterized. An initial value of 0.02 for simulations was chosen.

To provide some intuition, it is switched to a graph-theoretic perspective. The barcode 1-barcode 2 pairs can be modeled as edges on a bipartite graph formed by the set of barcode is (U, cardinality m₁) and the set of barcode 2s (V, cardinality m₂). Each patient sample is assigned a subset of cardinality k₁ from U and a subset of cardinality k₂ from V. If a particular barcode 1-barcode 2 pair is positive, the corresponding edge is part of the graph.

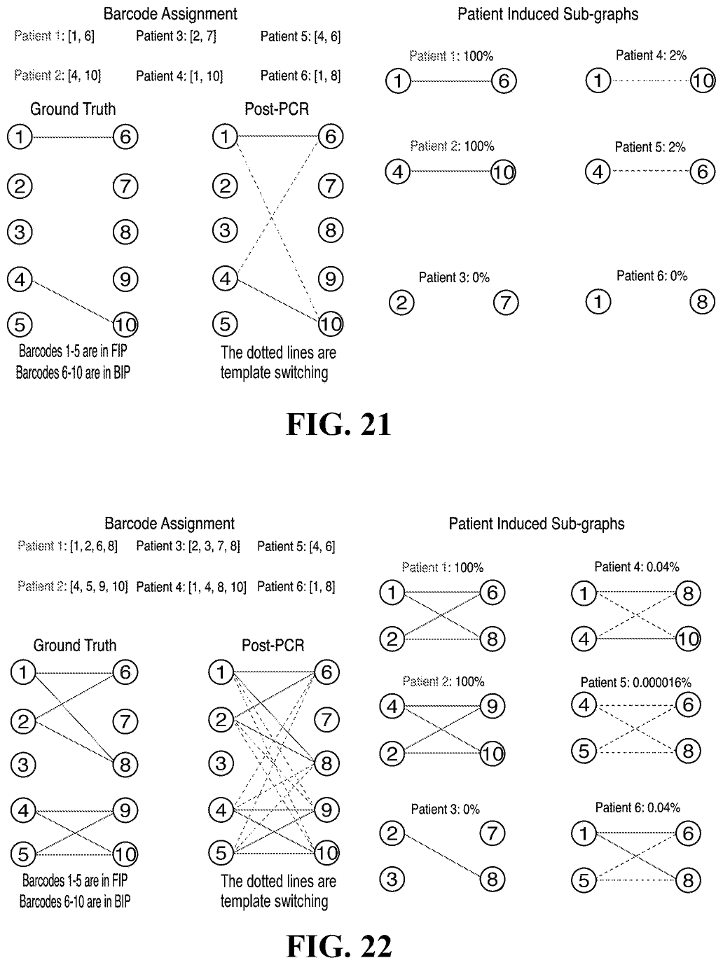

Template switching products then correspond to edges between any vertex in U with degree ≥1 and any vertex in V with degree ≥1. Each of these edges is added to the graph with probability Δ_{switch}. Inference for patient samples is performed on the final graph once all template switching edges have been considered. The status of a particular patient sample is inferred by considering the edges on the induced sub-graph formed by the patient sample's specific subsets. Examples of this for scenario 3 and scenario 5 are shown in FIGS. 21 and 22.

Each template switching product in scenario 3 leads to a false negative, as each patient sample corresponds to a single barcode 1-barcode 2 pair, which may be a concern. Scenarios 4 and 5 are able to mitigate this by requiring more than one positive barcode 1-barcode 2 pair per sample. However, the number of possible template switching products and the number of template switching products formed also increases in scenario 4 and 5. So, when positive samples are sparse, scenarios 4 and 5 may perform better than scenario 3, but when positive samples are very common, barcode saturation occurs and scenario 3 outperforms scenario 4 and 5.

We assumed that the number of barcodes used was small enough for physical validation of each barcode, so we set Δ_{synth}=0. The other error rates used were Δ_{stoch}=0.05 and Δ_{switch}=0.02, and it was assumed that 1% of patient samples were positive.

### 5 Numerical Simulations

Combining barcode loss and template switching, we ran numerical simulations to evaluate the performance of each scenario towards achieving our design goal. m₃=10 was chosen so 10 sub-batches per run. Simulations were run for 5000 or 500 iterations, for 100,000 samples per batch and 1,000,000 samples per batch respectively.

### 5.1 100,000 Patient Samples Per Batch

The initial design goal was to achieve FNPs and FPPs of <0.2% with a minimal number of total barcodes at 1% positive patient samples. With a small number of barcodes, scenario 3 (m1=m2=100) and scenario 4 (m₁=m₂=96, k₁=5, k′₁₂=3) do not achieve sufficient error rates.

Scenario 5 with m₁=m₂=96, k₁=k₂=3, under the second decoder scheme with k′₁₂=6 satisfies the design goal, suggesting that we can efficiently test 100,000 patient samples with a total of 96+96+10=202 barcodes. Increasing the number of barcode 1s and barcode 2s to 192 or 384 with k₁₂^{t}=5 lowers error rates further. See also Table 8.

TABLE 8
FPP/FNP with b = 100,000 m3 = 10, and p = 0.01 over 5000 interactions

| | | | | | | | Average | Average |
|---|---|---|---|---|---|---|---|---|
| Scenario | m₁, m₂ | k₁ | k₂ | k’₁₂ | k’ₗ | k’₂ | FNP | FPP |
| 3 | 100 | 1 | 1 | — | — | — | 0.049864 | 0.0075493 |
| 4 | 96 | 5 | 1 | 3 | — | — | 0.000808 | 0.0059579 |
| 5 | 96 | 3 | 3 | — | 2 | 2 | 0.000168 | 0.0041614 |
| 5 | 96 | 3 | 3 | 6 | — | — | 0.000448 | 0.0001958 |
| 5 | 192 | 3 | 3 | 5 | — | — | 0.000104 | 0.0000206 |
| 5 | 384 | 3 | 3 | 5 | — | — | 0.000104 | 0.0000004 |

### 5.2 1,000,000 Patient Samples Per Batch

It was also explored what number of barcodes would enable 1,000,000 samples per batch under the second decoder scheme with k₁=k₂=3 for scenario 5. A choice of m₁=m₂=192 does not suffice, but m₁=m₂=384 is sufficient with either k₁₂^{t}=5 or k′₁₂=6. This suggests that we can efficiently test 100,000 patient samples with a total of 384+384+10=778 barcodes. See Table 9.

TABLE 9
FPP/FNP with b = 1,000,000, m3 = 10, and
p = 0.01 over 500 iterations

| Scenario | m₁, m₂ | k₁, k₂ | k′₁₂ | Average FNP | Average FPP |
|---|---|---|---|---|---|
| 5 | 192 | 3 | 6 | 0.000232 | 0.0063356 |
| 5 | 384 | 3 | 6 | 0.000344 | 0.0000191 |
| 5 | 384 | 3 | 5 | 0.000016 | 0.0002789 |

6. Sample Skewing with Template Switching

Sample skewing errors could also occur, as discussed in Example 3. This variation could lead to over-representation of some positive samples, preventing detection of samples with lower viral abundance and giving rise to false negatives. This was modeled this in the same way as Example 3, adding the possibility of template switching.

### 6.1 Modeling Continuous Template Switching

The amount of template switching between two barcodes should be dependent on the number of molecules of each barcode present in the PCR pool. To incorporate this, we model the probability of forming a possible template switching product A-B as (Eq. 52)

$$Pr [ A - B ] = 4 {Δ}_{switch} σ ( \frac{1}{2} log ( Abundance ( A ) ) ) σ ( \frac{1}{2} log ( Abundance ( B ) ) ) \qquad ( 52 )$$

where Abundance(X) is the number of molecules containing barcode X/average number of molecules per positive barcode 1 or 2, and σ(x) is the sigmoid function

$$\frac{1}{1 + {e}^{- x}} .$$

This allows changes in template switching propensity for any given barcode 1-barcode 2 pair while still bounding the overall probability, with a 4-fold max increase. If a particular template switching product did form, the average of mean (barcode A molecules per barcode 2) and mean (barcode B molecules per barcode 1) molecules of A-B were added.

6.2 Numerical Simulations with Sample Skewing

For numerical simulations, the “Amplified” and “Saturated” models from e.g., Example 3 were used, with the same parameter values. It was continued to be assumed that each NextSeq run generates about 18 million reads per sub-batch. In the simulation, a given barcode pair was called as positive if the number of reads for the barcode pair (calculated by relative abundance multiplied by reads per sub-batch) was greater than or equal to a threshold oft reads. Otherwise, it was called as a negative barcode.

Using the optimized parameter values from section 5, we calculated the average FPPs and FNPs over either 500 or 5000 iterations for scenario 5, shown in Tables 10 or 11. All results are shown with error rates of Δ_{synth}=0, Δ_{stoch}⁼0.05, and Δ_{switch}=0.02.

TABLE 10
FPP/FNP with t = 100, m₃ = 10, k₁ = k₂ = 3,
and p = 0.01 over 5000 iterations

| Batch Size b | m₁, m₂ | k′₁₂ | Average FNP | Average FPP | Model |
|---|---|---|---|---|---|
| 100,000 | 96 | 6 | 0.318810 | 0.0000391 | Amplified |
| 100,000 | 192 | 5 | 0.318140 | 0.0000032 | Amplified |
| 100,000 | 384 | 5 | 0.317804 | 0.0000001 | Amplified |
| 100,000 | 96 | 6 | 0.000424 | 0.0002046 | Saturated |
| 100,000 | 192 | 5 | 0.000080 | 0.0000216 | Saturated |
| 100,000 | 384 | 5 | 0.000096 | 0.0000004 | Saturated |

TABLE 11
FPP/FNP with t = 100, m₃ = 10, k₁ = k₂ =
3, and p = 0.01 over 500 iterations

| Batch Size b | m₁, m₂ | k′₁₂ | Average FNP | Average FPP | Model |
|---|---|---|---|---|---|
| 1,000,000 | 384 | 5 | 0.665392 | 0.0000029 | Amplified |
| 1,000,000 | 384 | 6 | 0.665584 | 0.0000000 | Amplified |
| 1,000,000 | 384 | 5 | 0.000000 | 0.0002469 | Saturated |
| 1,000,000 | 384 | 6 | 0.000420 | 0.0000146 | Saturated |

Overall, the “Amplified” model renders these scenarios untenable, while the “Saturated” model maintains reasonable error rates. High FNPs arise in the “Amplified” model due to crowding out of samples with low viral load by samples with high viral load. It is expected that LAMP-seq to follow the “Saturated” model, suggesting that these scenarios are robust to sample skewing.

### 7. Code Availability

The scripts used to simulate and plot the models described here are available at the Github repository https://github.com/dbli2000/SARS-CoV2-Bloom-Filter.

### 8. Math Appendix

### 8.1 Scenario 4

In this scenario, we can view the barcode 2s and barcode 3s as splitting all of the samples into m₂·m₃ separate non-overlapping Bloom filters each with

$$\frac{b}{{m}_{2} {m}_{3}}$$

samples. Plugging these parameters into the equations for FPP_{Δk′, m2} and FNP_{Δk′,m2} from Example 3 gives the equations above.

### 8.2 Scenario 5

Each sample is now approximately contained in k₂ independent Bloom filters each with

$$\frac{{k}_{2} b}{{m}_{2} {m}_{3}}$$

samples, or

$$\frac{{k}_{1} {k}_{2} n}{{m}_{2} {m}_{3}}$$

positive barcode pairs. Solving for the error rate of each barcode pair using results from sections 2.2 and 2.3 of Example 3 gives the equations above.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A kit comprising:
a. amplification reagents
b. one or more primer sets comprising two or more primers, wherein at least one primer of at least one of the one or more primer sets comprises one or more barcodes, and wherein the primer sets are configured to amplify one or more target sequences from a sample in one or more amplification steps to generate amplicons that comprise the one or more target sequences and a unique combination of barcodes.

2. The kit of claim 1, wherein at least one of the one or more primer sets is configured for loop-mediated isothermal amplification (LAMP) or reverse transcription loop-mediated isothermal amplification (RT-LAMP) and comprises at least one forward inner primer (FIP), backward inner primer (BIP), or both.

3. The kit of claim 2, wherein one, two, or more primers of the at least one or more primer sets comprises one or more barcodes.

4. The kit of claim 2, wherein the barcode(s) is/are inserted between the two target-specific sequences of the FIP, the BIP, or both.

5. The kit of claim 2, wherein one or more of the one or more primer sets is configured for PCR amplification and wherein one or more primers of the one or more primer sets configured for PCR amplification comprises one or more PCR barcodes, sequencing adaptors, or both.

6. The kit of claim 1, wherein the individual barcodes are derived from a defined set of barcodes and configured such that individual barcodes are capable of being used in more than one kit and configured such that each kit receives a unique combination of barcodes.

7. The kit of claim 6, wherein the barcodes in the defined set of barcodes are selected to avoid barcodes having a sequence portion that is the reverse complement to the 3′ end of a forward inner primer (FIP) or a backward inner primer (BIP).

8. The kit of claim 6, wherein the number of individual barcodes used per kit is determined, at least in part, on a total number of barcode sequences in the defined set of barcodes, and a number of samples to be processed in parallel.

9. The kit of claim 8, wherein the number of barcodes is between 2 and 20.

10. The kit of claim 8, wherein the number of samples to be processed in parallel is optimized based on an expected or empirically determined fraction of positive samples, an estimated or empirically determined fraction of ineffective barcodes, a frequency of sample barcode dropout, a heterogeneity of sample representation in sequencing data, a false-positive cutoff rate, a false-negative cutoff rate, or a combination thereof.

11. The kit of claim 1, wherein at least two of the primers of at least one of the one or more primer sets are barcoded.

12. The kit of claim 1, wherein each barcode is between 4 and 40 bases in length.

13. The kit of claim 12, wherein each barcode is between 8 and 15 based in length.

14. The kit of claim 1, wherein the barcoded primers are included in the kit at equal concentrations.

15. The kit of any one of claims 1-14, wherein the amplification reagents are isothermal amplification reagents, polymerase chain reaction reagents, or both.

16. The kit of claim 15, wherein the isothermal amplification reagents are loop-mediated isothermal amplification (LAMP) or reverse transcription loop-mediated isothermal amplification (RT-LAMP) reagents.

17. The kit of any one of claims 1-16, further comprising a lysis reagent.

18. The kit of any one of claims 1-17, further comprising a control template DNA or RNA sequence.

19. The kit of any one of claims 1-18, wherein the kit further comprises a sample collection component.

20. The kit of claim 19, wherein the sample collection component is configured for collection of a nasal swab, an oral swab, a nasal wash, an oral wash, a fecal sample, a wound swab, or a combination thereof.

21. The kit of any one of claims 1-20, further comprising a sample dosing component.

22. The kit of any one of claims 1-21, further comprising a reaction vessel comprising a pre-mixed combination of amplification reagents and barcoded primers and configured to be sealed after receiving the sample, sample collection component, sample dosing component, or a combination thereof.

23. The kit of claim 22, wherein the reaction vessel is configured for use in an isothermal amplification reaction conducted at a point of care.

24. The kit of any one of claims 1-23, further comprising one or more heating components wherein the one or more heating components are configured for use in an isothermal reaction conducted at a temperature between 45° C. to 75° C.

25. The kit of claim 24, wherein the one or more chemical heating reaction reagents are configured for use in an isothermal reaction conducted at a temperature between 55° C. to 70° C.

26. The kit of claim 24, wherein the one or more heating components are configured for use in an isothermal reaction conducted at a temperature between 60° C. to 65° C.

27. The kit of any one of claims 24-26, wherein the one or more heating components is or includes one or more chemical heating reagents.

28. The kit of claim 27, wherein the one or more chemical heating reagents comprises molten sodium acetate.

29. The kit of any one of claims 1-27, wherein the one or more target sequences is used to genotype a subject, to detect a disease marker, detect an infectious agent, or a combination thereof.

30. The kit of any one of claims 1-28, further comprising controls primers that are configured to amplify a target sequence of an endogenous RNA of the sample to confirm successful sample collection.

31. The kit of any one of claims 1-29, further comprising a colorimetric or turbidimetric indicator.

32. The kit of claim 28, wherein the infectious agent is a viral agent.

33. The kit of claim 31, wherein the viral agent is an RNA virus.

34. The kit of claim 32, wherein the RNA virus is a coronavirus.

35. The kit of claim 33, wherein the coronavirus is SARS-CoV2.

36. A method of parallel detection of one or more target sequences across multiple samples, comprising:
e. separating a set of samples into one or more pooled sample sets, wherein each sample comprises an initial amplicon comprising one or more target sequences and at least one barcode;
f. conducting an amplification reaction on the one or more pooled sample sets to further amplify the amplicons, and optionally further adding an additional barcode to the amplicon;
g. sequencing the amplicons; and
h. identifying individual samples from the pooled sample set that are positive for the one or more target sequences based on sequencing of the amplicons, wherein identification is based, at least in part, on detection of the unique combination of barcodes.

37. The method of claim 36, wherein the amplicons in each individual sample comprising the set of samples of step (a) are generated by conducting an isothermal amplification reaction on each individual sample using one or more primer sets and wherein a primer in each primer set comprises a barcode and each set of primers comprises a combination of barcodes unique to each sample.

38. The method of claim 37, wherein the number of barcodes used per sample is determined, at least in part, on the total number of barcode sequences in a defined set of barcode sequences and a number of samples to be processed in parallel.

39. The method of claim 38, wherein the number of barcodes used per sample is between 2 and 20.

40. The method of claim 36, wherein barcodes are selected so as to avoid barcodes comprising a sequence portion that is a reverse complement to the 3′ end of a primer, in particular the 3′ end of a forward inner primer (FIP).

41. The method of claim 36, wherein the number of samples to be processed in a pooled set is optimized based on an expected or empirically determined fraction of positive samples, an estimated or empirically determined fraction of ineffective barcodes, a frequency of sample barcode dropout, a heterogeneity of sample representation in sequencing data, a false-positive cutoff rate, a false-negative cutoff rate, or a combination thereof.

42. The method of claim 36, wherein the number of pooled sets is 1-11, 12-96, or 384.

43. The method of claim 37, wherein the isothermal amplification reaction is loop-mediated isothermal amplification (LAMP) or reverse transcription loop-mediated isothermal amplification (RT-LAMP).

44. The method of claim 37, wherein the barcode sequences are inserted between two target-specific sequences of either a forward inner primer (FIP), a backward inner primer (BIP), or both.

45. The method of any one of claims 36-44, wherein the samples are further heat-inactivated either prior to or after being pooled into a pooled sample set.

46. The method of claim 36, wherein the amplicon resulting from amplification of the pooled sets spans a fraction of the target nucleic acid sequence not covered or only partially covered by the primers used to generate an initial amplicon.

47. The method of claim 36, wherein the amplicon resulting from the amplification of pooled sets spans one or both of the junctions between a barcode sequence and the target nucleic acid sequence.

48. The method of claim 36, wherein sequencing the amplicons comprises deep sequencing of the amplicons.

49. The method of claim 36, wherein the set of samples is diluted to between 1:1,000 to 1:1,000,000 prior to the amplification reaction of (b).
